(19) United States
(12) Patent Application Publication
(10) Pub. No.: US 2008/0241918 A1
(43) Pub. Date: Oct. 2, 2008

(54) Hemagglutinin polypeptides, and reagents and methods relating thereto

(76) Inventors: Ram Sasisekharan, Cambridge, MA (US); Karthik Viswanathan, Cambridge, MA (US); Aarthi Chandrasekaran, Cambridge, MA (US); Rahul Raman, Cambridge, MA (US); Aravind Srinivasan, Cambridge, MA (US); S. Raguram, Hillsborough, NJ (US); Viswanathan Sasisekharan, Cambridge, MA (US)

Correspondence Address: CHOATE, HALL & STEWART LLP, TWO INTERNATIONAL PLACE, BOSTON, MA, 02110, US

(21) Appl. No.: 11/893,171

(22) Filed: Aug. 14, 2007

Related U.S. Application Data
(60) Provisional application No. 60/837,868, filed on Aug. 14, 2006.
(60) Provisional application No. 60/837,869, filed on Aug. 14, 2006.

(51) Int. Cl. C12N 5/06; C07K 14/00; C12N 15/11; C07K 16/18; C12N 15/00

(52) U.S. Cl. 435/325; 530/380; 536/23.5; 435/320.1; 530/387.1

(57) ABSTRACT

The present invention provides a system for analyzing interactions between glycans and interaction partners that bind to them. The present invention also provides HA polypeptides that bind to umbrella-topology glycans, and reagents and methods relating thereto.

### PRIORITY CLAIM

The present application claims priority under 35 USC 119(e) to co-pending U.S. Provisional patent application Ser. No. 60/837,868, filed on Aug. 14, 2006, and to co-pending U.S. provisional patent application Ser. No. 60/837,869, filed on Aug. 14, 2006. The entire contents of each of these prior applications is incorporated herein by reference.

### GOVERNMENT SUPPORT

This invention was made with United States government support awarded by the National Institute of General Medical Sciences under contract number U54 GM62116 and by the National Institutes of Health under contract number GM57073. The United States Government has certain rights in the invention.

### BACKGROUND OF THE INVENTION

Influenza has a long history of pandemics, epidemics, resurgences and outbreaks. Avian influenza, including the H5N1 strain, is a highly contagious and potentially fatal pathogen, but it currently has only a limited ability to infect humans. However, avian flu viruses have historically observed to accumulate mutations that alter its host specificity and allow it to readily infect humans. In fact, two of the major flu pandemics of the last century originated from avian flu viruses that changed their genetic makeup to allow for human infection.

There is a significant concern that the current H5N1, H7N7, H9N2 and H2N2 avian influenza strains might accumulate mutations that alter their host specificity and allow them to readily infect humans. Therefore, there is a need to assess whether the HA protein in these strains can, in fact, convert to a form that can readily infect humans, and a further need to identify HA variants with such ability. There is a further need to understand the characteristics of HA proteins generally that allow or prohibit infection of different subjects, particularly humans. There is also a need for vaccines and therapeutic strategies for effective treatment or delay of onset of disease caused by influenza virus.

### SUMMARY OF THE INVENTION

The present invention provides hemagglutinin polypeptides with particular glycan binding characteristics. In particular, the present invention provides hemagglutinin polypeptides that bind to sialylated glycans having an umbrella-like topology. In certain embodiments, inventive HA polypeptides bind to umbrella glycans with high affinity and/or specificity. In some embodiments, inventive HA polypeptides show a binding preference for umbrella glycans as compared with cone-topology glycans.

The present invention also provides diagnostic and therapeutic reagents and methods associated with provided hemagglutinin polypeptides, including vaccines.

### BRIEF DESCRIPTION OF THE DRAWING

FIG. 1. Alignment of exemplary sequences of wild type HA. Sequences were obtained from the NCBI influenza virus sequence database (http://www.ncbi.nlm.nih.gov/genomes/FLU/FLU.html)

FIG. 2. Sequence alignment of HA glycan binding domain. Gray: conserved amino acids involved in binding to sialic acid. Red: particular amino acids involved in binding to Neu5Acα2-3/6Gal motifs. Yellow: amino acids that influence positioning of Q226 (137, 138) and E190 (186, 228). Green: amino acids involved in binding to other monosaccharides (or modifications) attached to Neu5Acα2-3/6Gal motif. The sequence for ASI30, APR34, ADU63, ADS97 and Viet04 were obtained from their respective crystal structures. The other sequences were obtained from SwissProt (http://us.expasy.org). Abbreviations: ADA76, A/duck/Alberta/35/76 (H1N1); ASI30, A/Swine/Iowa/30 (H1N1); APR34, A/Puerto Rico/8/34 (H1N1); ASC18, A/South Carolina/1/18 (H1N1), AT91, A/Texas/36/91 (H1N1); ANY18, A/New York/1/18 (H1N1); ADU63, A/Duck/Ukraine/1/63 (H3N8); AAI68, A/Aichi/2/68 (H3N2); AM99, A/Moscow/10/99 (H3N2); ADS97, A/Duck/Singapore/3/97 (H5N3); Viet04, A/Vietnam/1203/2004 (H5N1).

FIG. 3. Sequence alignment illustrating conserved subsequences characteristic of H1 HA.

FIG. 4. Sequence alignment illustrating conserved subsequences characteristic of H3 HA.

FIG. 5. Sequence alignment illustrating conserved subsequences characteristic of H5 HA.

FIG. 6. Framework for understanding glycan receptor specificity. α2-3- and/or α2-6-linked glycans can adopt different topologies. According to the present invention, the ability of an HA polypeptide to bind to certain of these topologies confers upon it the ability to mediate infection of different hosts, for example, humans. As illustrated in this figure, the present invention defines two particularly relevant topologies, a “cone” topology and an “umbrella” topology. The cone topology can be adopted by α2-3- and/or α2-6-linked glycans, and is typical of short oligosaccharides or branched oligosaccharides attached to a core (although this topology can be adopted by certain long oligosaccharides). The umbrella topology can only be adopted by α2-6-linked glycans (presumably due to the increased conformational plurality afforded by the extra C5-C6 bond that is present in the α2-6 linkage), and is predominantly adopted by long oligosaccharides or branched glycans with long oligosaccharide branches, particularly containing the motif Neu5Acα2-6Galβ1-3/4GlcNAc-. As described herein, ability of HA polypeptides to bind the umbrella glycan topology, confers binding to human receptors and/or ability to mediate infection of humans.

FIG. 7. Interactions of HA residues with cone vs umbrella glycan topologies. Analysis of HA-glycan co-crystals reveals that the position of Neu5Ac relative to the HA binding site is almost invariant. Contacts with Neu5Ac involve highly conserved residues such as F98, S/T136, W153, H183 and L/1194. Contacts with other sugars involve different residues, depending on whether the sugar linkage is α2-3 or α2-6 and whether the glycan topology is cone or umbrella. For example, in the cone topology, the primary contacts are with Neu5Ac and with Gal sugars. E190 and Q226 play particularly important roles in this binding. This Figure also illustrates other positions (e.g., 137, 145, 186, 187, 193, 222) that can participate in binding to cone structures. In some cases, different residues can make different contacts with different glycan structures. The type of amino acid in these positions can influence ability of an HA polypeptide to bind to receptors with different modification and/or branching patterns in the glycan structures. In the umbrella topology, contacts are made with sugars beyond Neu5Ac and Gal. This Figure illustrates residues (e.g., 137, 145, 156, 159, 186, 187, 189, 190, 192, 193, 196, 222, 225, 226) that can participate in binding to umbrella structures. In some cases, different residues can make different contacts with different glycan structures. The type of amino acid in these positions can influence ability of an HA polypeptide to bind to receptors with different modification and/or branching patterns in the glycan structures. In some embodiments, a D residue at position 190 and/or a D residue at position 225 contribute(s) to binding to umbrella topologies.

FIG. 8. Exemplary cone topologies. This Figure illustrates certain exemplary (but not exhaustive) glycan structures that adopt cone topologies.

FIG. 9. Exemplary umbrella topologies. This Figure illustrates certain exemplary (but not exhaustive) glycan structures that adopt umbrella topologies.

FIG. 10. Glycan profile of human bronchial epithelial cells and human colonic epithelial cells. To further investigate the glycan diversity in the upper respiratory tissues, N-linked glycans were isolated from HBEs (a representative upper respiratory cell line) and analyzed using MALDI-MS. The predominant expression of a2-6 in HBEs was confirmed by pre-treating the sample with Sialidase S (a2-3 specific) and Sialidase A (cleaves and SA). The predominant expression of glycans with long branch topology is supported by TOF-TOF fragmentation analysis of representative mass peaks (highlighted in cyan). To provide a reference for glycan diversity in the upper respiratory tissues, the N-linked glycan profile of human colonic epithelial cells (HT29; a representative gut cell line) was obtained. This cel line was chosen because the current H5N1 viruses have been shown to infect gut cells. Sialidase A and S pre-treatment controls showed predominant expression of a2-3 glycans (highlighted in red) in the HT-29 cells. Moreover, the long branch glycan topology is not as prevalent as observed for HBEs. Therefore, human adaptation of the H5N1 HA would involve HA mutations that would enable high affinity binding to the diverse glycans expressed in the human upper respiratory tissues (e.g., umbrella glycans).

FIG. 11. Data mining platform. Shown in (A) are the main components of the data mining platform. The features are derived from the data objects which are extracted from the database. The features are prepared into datasets that are used by the classification methods to derive patterns or rules (B), shows the key software modules that enable the user to apply the data mining process to the glycan array data.

FIG. 12. Features used in data mining analysis. This figure shows the features defined herein as representative motifs that illustrate the different types of pairs, triplets and quadruplets abstracted from the glycans on the glycan microarray. The rationale behind choosing these features is based on the binding of di-tetra saccharides to the glycan binding site of HA. The final dataset comprise features from the glycans as well as the binding signals for each of the HAs screened on the array. Among the different methods for classification, the rule induction classification method was utilized. One of the main advantages of this method is that it generates IF-THEN rules which can be interpreted more easily when compared to the other statistical or mathematical methods. The two main objectives of the classification were: (1) identifying features present on a set of high affinity glycan ligands, which enhance binding, and (2) identifying features that are in the low affinity glycan ligands that are not favorable for binding.

FIG. 13. Classifiers used in data mining analysis. This figure presents a table of classifier ids and rules.

FIG. 14. Conformational map and solvent accessibility of Neu5Acα2-3Gal and Neu5Acα2-6Gal motifs. Panel A shows the conformational map of Neu5Acα2-3Gal linkage. The encircled region 2 is the trans conformation observed in the APR34_H1_{—}23, ADU63_H3_{—}23 and ADS97_H5_{—}23 co-crystal structures. The encircled region 1 is the conformation observed in the AAI68_H3_{—}23 co-crystal structure. Panel B shows the conformational map of Neu5Acα2-6Gal where the cis-conformation (encircled region 3) is observed in all the HA-α2-6 sialylated glycan co-crystal structures. Panel C shows difference between solvent accessible surface area (SASA) of Neu5Ac α2-3 and α2-6 sialylated oligosaccharides in the respective HA-glycan co-crystal structures. The red and cyan bars respectively indicate that Neu5Ac in α2-6 (positive value) or α2-3 (negative value) sialylated glycans makes more contact with glycan binding site. Panel D shows difference between SASA of NeuAc in α2-3 sialylated glycans bound to swine and human H1 (H1_{α2-3}), avian and human H3 (H3_{α2-3}), and of NeuAc in α2-6 sialylated glycans bound to swine and human H1 (H1_{α2-6}). The negative bar in cyan for H3_{α2-3} indicates lesser contact of the human H3 HA with Neu5Acα2-3Gal compared to that of avian H3. Torsion angles—φ: C2-C1-O-C3 (for Neu5Acα2-3/6 linkage); ψ: C1-O-C3-H3 (for Neu5Acα2-3Gal) or C1-O-C6-C5 (for Neu5Acα2-6Gal); ω: O-C6-C5-H5 (for Neu5Acα2-6Gal) linkages. The φ, ψ maps were obtained from GlycoMaps DB (http://www.glycosciences.de/modeling/glycomapsdb/) which was developed by Dr. Martin Frank and Dr. Claus-Wilhelm von der Lieth (German Cancer Research Institute, Heidelberg, Germany). The coloring scheme from high energy to low energy is from bright red to bright green, respectively.

FIG. 15. Residues involved in binding of H1, H3 and H5 HA to α2-3/6 sialylated glycans. Panels A-D show the difference (Δ in the abscissa) in solvent accessible surface area (SASA) of residues interacting with α2-3 and α2-6 sialylated glycans, respectively, in ASI30_H1, APR34_H1, ADU63_H3 and ADS97_H5 co-crystal structures. Green bars correspond to residues that directly interact with the glycan and light orange bars correspond to residues proximal to Glu/Asp190 and Gln/Leu226. Positive value of Δ for the green bars indicates more contact of that residue with α2-6 sialylated glycans whereas a negative value of Δ indicates more contact with α2-3 sialylated glycans. Panel E summarizes in tabular form the residues involved in binding to α2-3/6 sialylated glycans in H1, H3 and H5 HA. Certain key residues involved in binding to α2-3 sialylated glycans are colored blue and certain key residues involved in binding to α2-6 sialylated glycans are colored red.

FIG. 16. Binding of Viet04_H5 HA to biantennary α2-6 sialylated glycan (cone topology). Stereo view of surface rendered Viet04_H5 glycan binding site with Neu5Acα2-6Gal linkage in the extended conformation (obtained from the pertussis toxin co-crystal structure; PDB ID: 1PTO). Lys193 (orange) does not have any contacts with the glycan in this conformation. The additional amino acids potentially involved in binding to the glycan in this conformation are Asn186, Lys222 and Ser227. However, certain contacts observed in the HA binding to the α2-6 sialylated oligosaccharide in the cis-conformation are absent in the extended conformation. Without wishing to be bound by any particular theory, we note that this suggests that the extended conformation may not bind to HA as optimally as the cis-conformation. The structures of branched N-linked glycans where the Neu5Acα2-6Galβ1-4GlcNAcb branch was attached to the Manα1-3Man (PDB ID: 1LGC) and Manα1-6Man (PDB ID: 1ZAG) were superimposed on to the Neu5Acα2-6Gal linkage in the Viet04_H5 HA binding site for both the cis and the extended conformation of this linkage. The superimposition shows that the structure with Neu5Acα2-6Galβ1-4GlcNAc branch attached to Manα1-6Man of the core has unfavorable steric overlaps with the binding site (in both the conformations). On the other hand, the structure with this branch attached to Manα1-3Man of the core (shown in figure where trimannose core is colored in purple) has steric overlaps with Lys193 in the cis-conformation but can bind without any contact with Lys193 in the extended conformation, albeit less optimally.

FIG. 17. Production of WT H1, H3 and H5 HA. Panel A shows the soluble form of HA protein from H1N1 (A/South Carolina/1/1918), H3N2 (A/Moscow/10/1999) and H5N1 (A/Vietnam/1203/2004), run on a 4-12% SDS-polyacrylaminde gel and blotted onto nitrocellulose membranes. H1N1 HA was probed using goat anti-Influenza A antibody and anti-goat IgG-HRP. H3N2 was prodes using ferret anti-H3N2 HA antisera and anti-ferret-HRP. H5N1 was probed using anti-avian H5N1 HA antibody and anti-rabbit IgG-HRP. H1N1 HA and H3N2 HA are present as HA0, while H5N1 HA is present as both HA0 and HA1. Panel B shows full length H5N1 HA and two variants (Glu190Asp, Lys193Ser, Gly225Asp, Gln226Leu, “DSDL” and GLu190Asp Lys193Ser Gln223Leu Gly228Ser “DSLS”) run on an SDS-polyacrylamide gel and blotted onto a nitrocellulose membrane. The HA was probed with anti-avian H5N1 antibody and anti-rabbit IgG-HRP.

FIG. 18. Lectin staining of upper respiratory tissue sections. A co-stain of the tracheal tissue with Jacalin (green) and ConA (red) reveals a preferential binding of Jacalin (binds specifically to O-linked glycans) to goblet cells on the apical surface of the trachea and conA (binds specifically to N-linked glycans) to the ciliated tracheal epithelial cells. Without wishing to be bound by any particular theory, we note that this finding suggests that goblet cells predominantly express O-linked glycans while ciliated epithelial cells predominantly express N-linked glycans. Co-staining of trachea with Jacalin and SNA (red; binds specifically to α2-6) shows binding of SNA to both goblet and ciliated cells. On the other hand, co-staining of Jacalin (green) and MAL (red), which specifically binds to α2-3 sialylated glycans, shows weak minimal to no binding of MAL to the pseudostratified tracheal epithelium but extensive binding to the underlying regions of the tissue. Together, the lectin staining data indicated predominant expression and extensive distribution of α2-6 sialylated glycans as a part of both N-linked and O-linked glycans respectigely in ciliated and goblet cells on the apical side of the tracheal epithelium.

FIG. 19. Dose response binding of recombinant H1, H3 WT HA to upper and lower respiratory tissue sections. HA binding is shown in green against propidium iodide staining (red). The apical side of tracheal tissue predominantly expresses α2-6 glycans with long branch topology. The alveolar tissue on the other hand predominantly expresses a2-3 glycans. H1 HA binds significantly to the apical surface of the trachea and its binding reduces gradually with dilution from 40 to 10 ug/ml. H1 HA also shows some weak binding to the alveolar tissue only at the highest concentration. The binding pattern of H3 HA is different from that of H1 HA. For example, H3 HA shows significant binding to both tracheal and alveolar tissue sections at 40 and 20 ug/ml. However, at a concentration of 10 ug/ml, H3 HA shows binding primarily to the apical side of the tracheal tissue and little or no binding to the alveolar tissue. Together, these tissue binding data highlight the importance of high affinity binding to the apical side of tracheal tissue. Furthermore, these data reveal that high specificity for α2-6 sialylated glycan (as demonstrated by H1 HA) is not absolutely required to mediate infection of humans, since H3 HA shows some affinity for α2-3 sialylated glycans.

FIG. 20. Direct binding dose response of H1, H3 and H5 WT HA. Shows from top to bottom are the binding signals (normalized to the saturation level of around 800000) respectively for wild type H1, H3, and H5 HA at various concentrations. The legend for the glycans is shown as an inset, where LN corresponds to Galb104GlcNAc and 3′SLN and 6′SLN, respectively, correspond to α2-3 and α2-6 linked sialic acid at the LN. The characteristic binding pattern of the H1 and H3 HAs, which are adapted to infect humans, is their biding at saturating levels to the long α2-6 (6′SLN-LN) glycans over a range of dilution from 40 ug/ml down to 5 ug/ml. While H1 HA is highly specific for binding to the long α2-6 sialylated glycans, H3 HA also binds to short α2-6 sialylated glycans (6′SLN) with high affinity and to a long α2-3 with lower affinity relative to α2-6. This direct binding dose response of H1 and H3 HA is consistent with the tissue binding pattern. Furthermore, the high affinity binding of H1 and H3 HA to long α2-6 silalylated glycans correlates with their extensive binding to the apical side of tracheal tissues (which expresses α2-6 sialylated glycans with long branch topology). This correlation provides valuable insights into the upper respiratory tissue tropism of human-adapted H1 and H3 Has. The H5 HA, on the other hand, shows the opposite glycan binding trend, binding with high affinity to α2-3 (saturating signals from 40 ug/ml down to 2.5 ug/ml) as compared with its relatively low affinity for α2-6 sialylated glycans (significant signals seen only at 20-40 ug/ml). Thus, without wishing to be bound by any particular theory, the present inventors propose that a necessary condition for human adaptation of an HA polypeptide (e.g., avian H5 HA) is to gain the ability to bind to long α2-6 sialylated glycans (e.g., umbrella topology glycans), which are predominantly expressed in the human upper airway, with high affinity.

### DESCRIPTION OF HA SEQUENCE ELEMENTS

### HA Sequence Element 1

HA Sequence Element 1 is a sequence element corresponding approximately to residues 97-185 (where residue positions are assigned using H3 HA as reference) of many HA proteins found in natural influenza isolates. This sequence element has the basic structure:

C(Y/F)PX₁CX₂WX₃WX₄HHP, wherein:
X₁ is approximately 30-45 amino acids long;
X₂ is approximately 5-20 amino acids long;
X₃ is approximately 25-30 amino acids long; and
X₄ is approximately 2 amino acids long.

In some embodiments, X₁ is about 35-45, or about 35-43, or about 35, 36, 37, 38, 38, 40, 41, 42, or 43 amino acids long. In some embodiments, X₂ is about 9-15, or about 9-14, or about 9, 10, 11, 12, 13, or 14 amino acids long. In some embodiments, X₃ is about 26-28, or about 26, 27, or 28 amino acids long. In some embodiments, X₄ has the sequence (G/A) (I/V). In some embodiments, X₄ has the sequence GI; in some embodiments, X₄ has the sequence GV; in some embodiments, X₄ has the sequence AI; in some embodiments, X₄ has the sequence AV. In some embodiments, HA Sequence Element 1 comprises a disulfide bond. In some embodiments, this disulfide bond bridges residues corresponding to positions 97 and 139 (based on the canonical H3 numbering system utilized herein).

In some embodiments, and particularly in H1 polypeptides, X₁ is about 43 amino acids long, and/or X₂ is about 13 amino acids long, and/or X₃ is about 26 amino acids long. In some embodiments, and particularly in H1 polypeptides, HA Sequence Element 1 has the structure:

CYPX_{1A}T(A/T)(A/S)CX₂WX₃WX₄HHP, wherein:

X_{1A} is approximately 27-42, or approximately 32-42, or approximately 32-40, or approximately 26-41, or approximately 31-41, or approximately 31-39, or approximately 31, 32, 33, 34, 35, 36, 37, 38, 39, or 40 amino acids long, and X₂-X₄ are as above.

In some embodiments, and particularly in H1 polypeptides, HA Sequence Element 1 has the structure:

CYPX_{1A}T(A/T)(A/S)CX₂W(I/L)(T/V)X_{3A}WX₄HHP, wherein:

X_{1A} is approximately 27-42, or approximately 32-42, or approximately 32-40, or approximately 32, 33, 34, 35, 36, 37, 38, 39, or 40 amino acids long,

X_{3A} is approximately 23-28, or approximately 24-26, or approximately 24, 25, or 26 amino acids long, and X₂ and X₄ are as above.

In some embodiments, and particularly in H1 polypeptides, HA Sequence Element 1 includes the sequence:

QLSSISSFK,

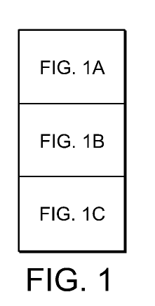

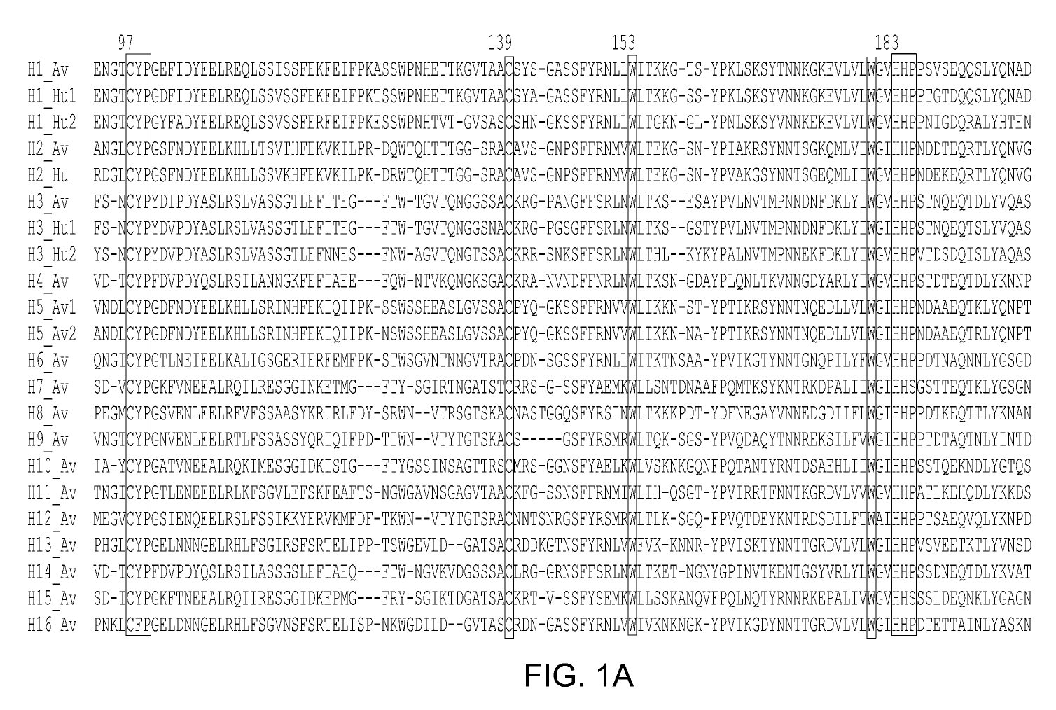

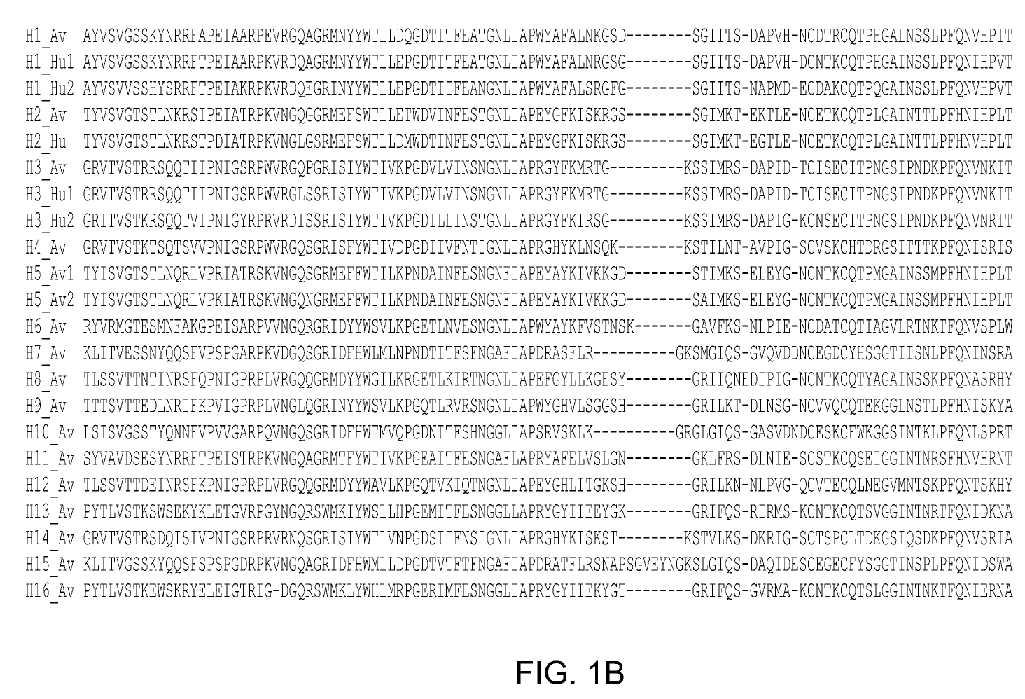

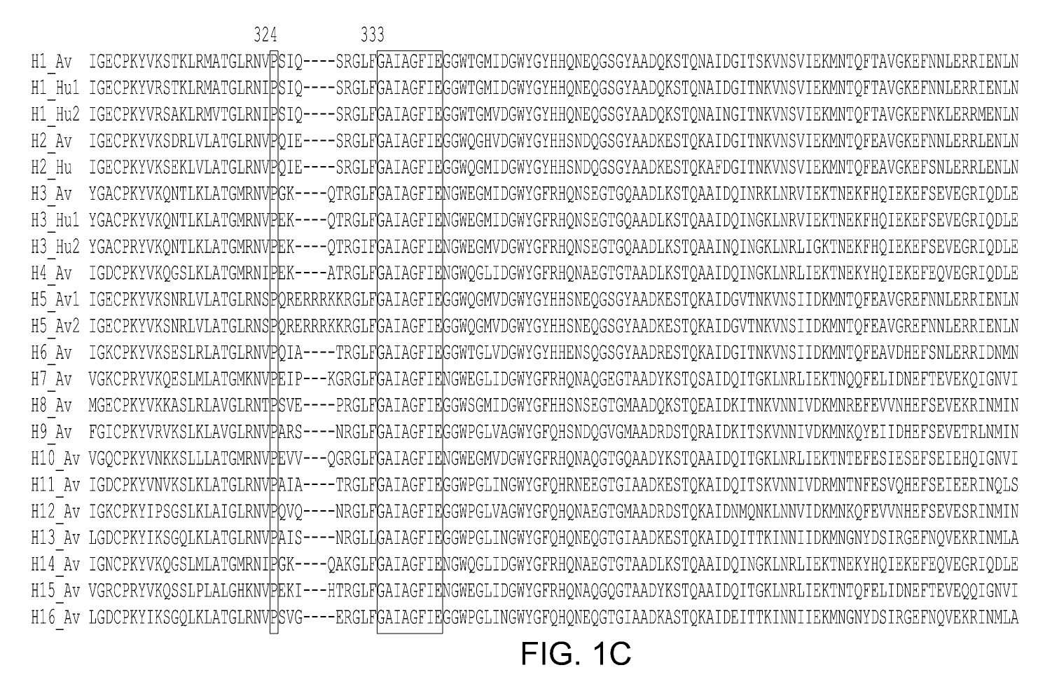

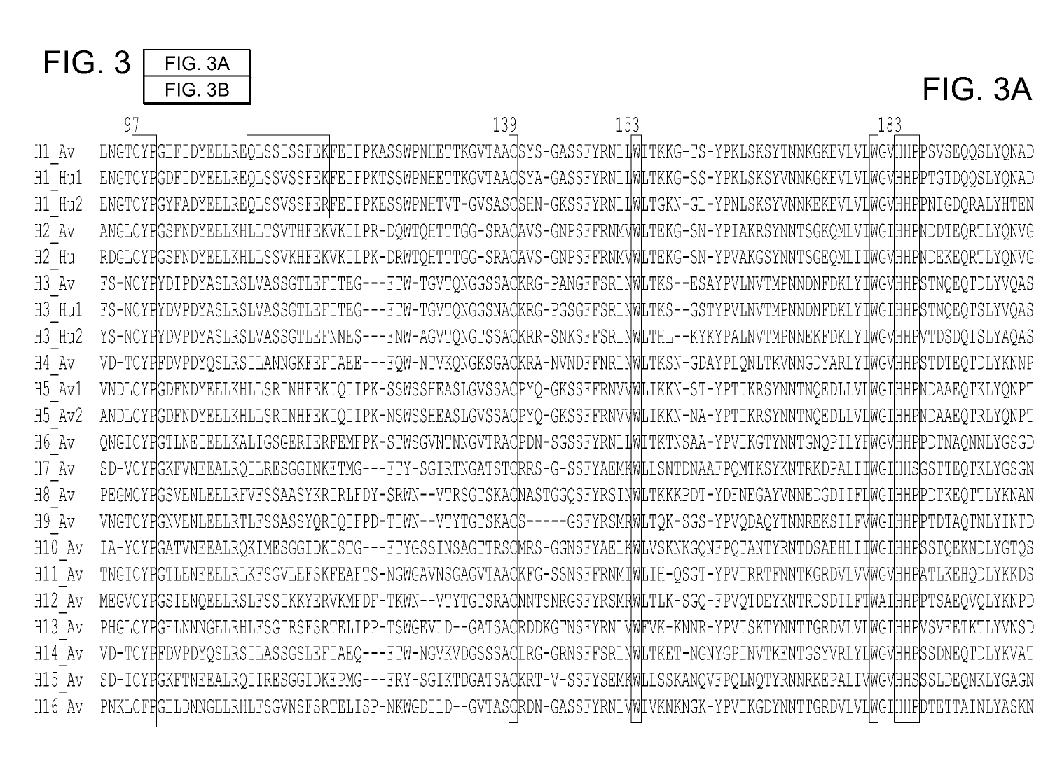

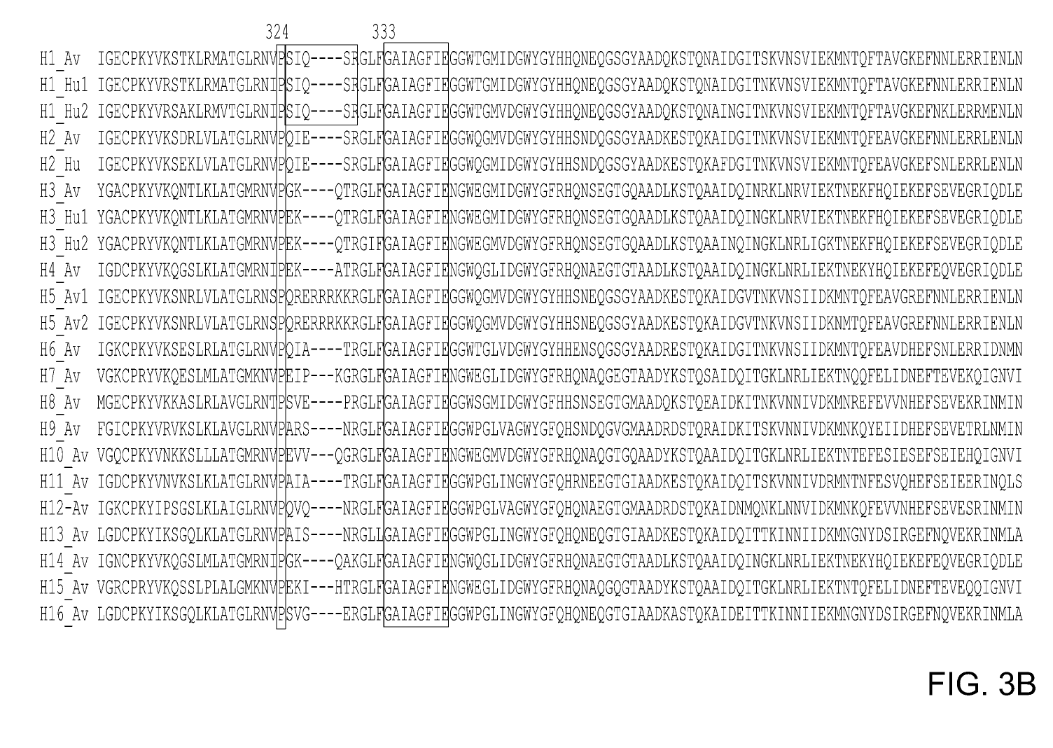

typically within X₁, (including within X_{1A}) and especially beginning about residue 12 of X₁ (as illustrated, for example, in FIGS. 1-3).

In some embodiments, and particularly in H3 polypeptides, X₁ is about 39 amino acids long, and/or X₂ is about 13 amino acids long, and/or X₃ is about 26 amino acids long.

In some embodiments, and particularly in H3 polypeptides, HA Sequence Element 1 has the structure:

CYPX_{1A}S(S/N)(A/S)CX₂WX₃WX₄HHP, wherein:

X_{1A} is approximately 27-42, or approximately 32-42, or approximately 32-40, or approximately 23-38, or approximately 28-38, or approximately 28-36, or approximately 28, 29, 30, 31, 32, 33, 34, 35, 36, 37, 38, 39, or 40 amino acids long, and X₂-X₄ are as above.

In some embodiments, and particularly in H3 polypeptides, HA Sequence Element 1 has the structure:

CYPX_{1A}S(S/N)(A/S)CX₂WL(T/H)X_{3A}WX₄HHP, wherein:

X_{1A} is approximately 27-42, or approximately 32-42, or approximately 32-40, or approximately 32, 33, 34, 35, 36, 37, 38, 39, or 40 amino acids long,

X_{3A} is approximately 23-28, or approximately 24-26, or approximately 24, 25, or 26 amino acids long, and X₂ and X₄ are as above.

In some embodiments, and particularly in H3 polypeptides, HA Sequence Element 1 includes the sequence:

(L/I)(V/I)ASSGTLEF,

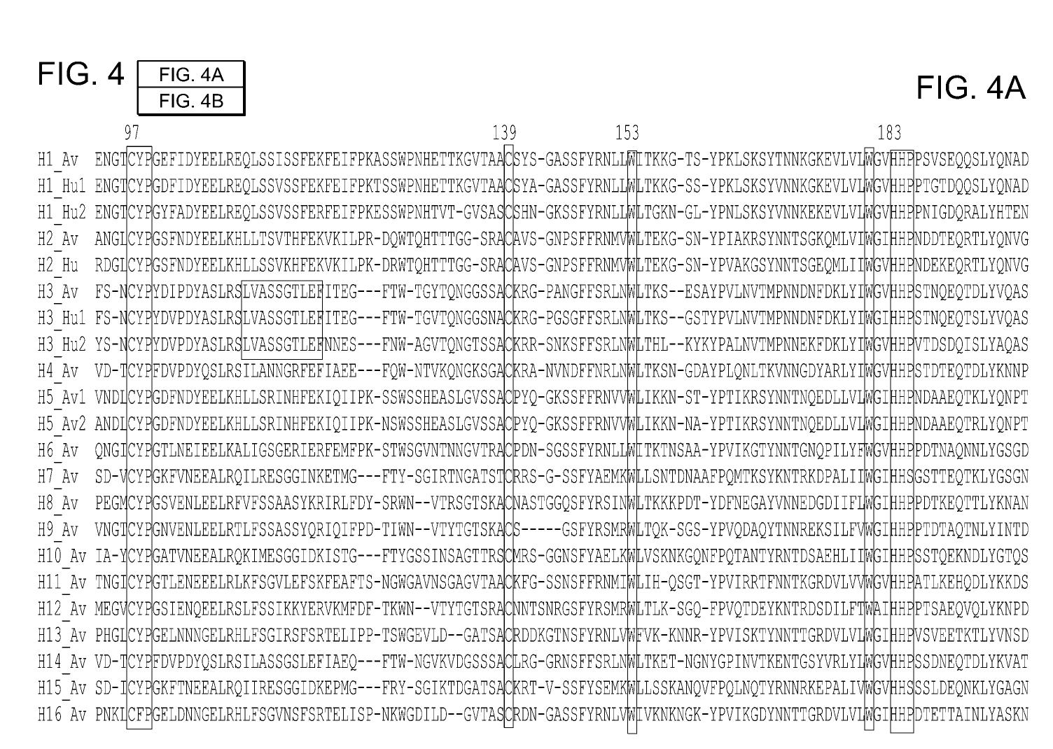

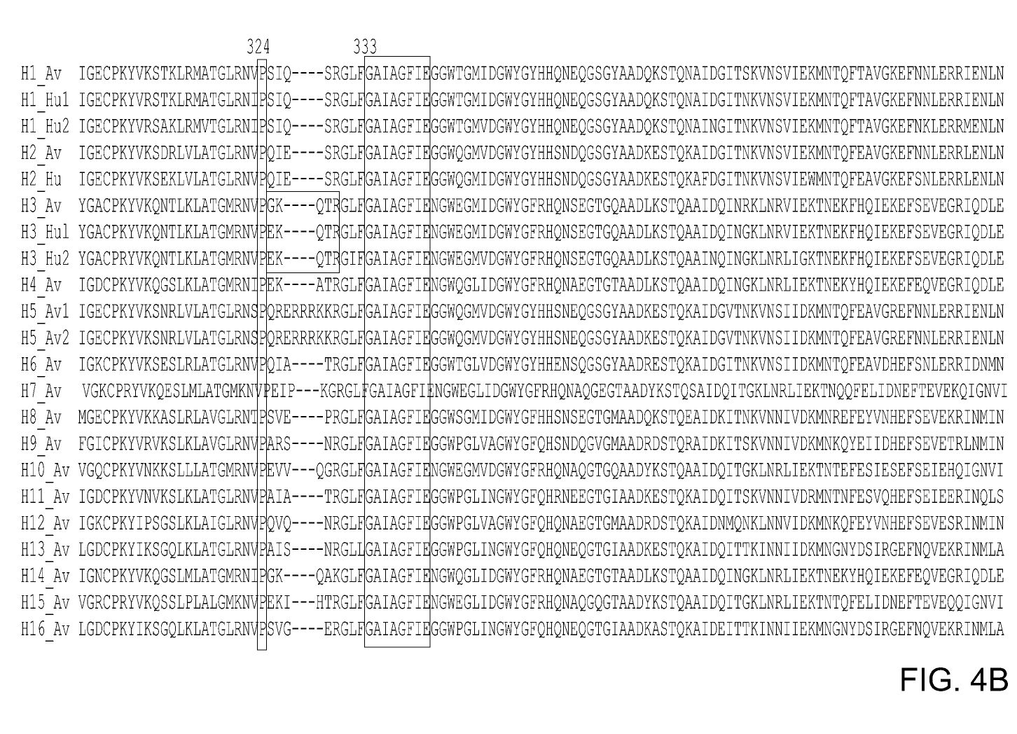

typically within X₁ (including within X_{1A}), and especially beginning about residue 12 of X₁ (as illustrated, for example, in FIGS. 1, 2 and 4).

In some embodiments, and particularly in H5 polypeptides, X₁ is about 42 amino acids long, and/or X₂ is about 13 amino acids long, and/or X₃ is about 26 amino acids long.

In some embodiments, and particularly in H5 polypeptides, HA Sequence Element 1 has the structure:

CYPX_{1A}SSACX₂WX₃WX₄HHP, wherein:

X_{1A} is approximately 27-42, or approximately 32-42, or approximately 32-40, or approximately 23-38, or approximately 28-38, or approximately 28-36, or approximately 28, 29, 30, 31, 32, 33, 34, 35, 36, 37, 38, 39, or 40 amino acids long, and X₂-X₄ are as.

In some embodiments, and particularly in H5 polypeptides, HA Sequence Element 1 has the structure:

CYPX_{1A}SSACX₂WLIX_{3A}WX₄HHP, wherein:

X_{1A} is approximately 27-42, or approximately 32-42, or approximately 32-40, or approximately 32, 33, 34, 35, 36, 37, 38, 39, or 40 amino acids long, and

X_{3A} is approximately 23-28, or approximately 24-26, or approximately 24, 25, or 26 amino acids long, and X₂ and X₄ are as above.

In some embodiments, and particularly in H5 polypeptides, HA Sequence Element 1 is extended (i.e., at a position corresponding to residues 186-193) by the sequence:

NDAAEXX(K/R)

In some embodiments, and particularly in H5 polypeptides, HA Sequence Element 1 includes the sequence:

YEELKHLXSXXNHFEK,

typically within X₁, and especially beginning about residue 6 of X₁ (as illustrated, for example, in FIGS. 1, 2, and 5).

### HA Sequence Element 2

HA Sequence Element 2 is a sequence element corresponding approximately to residues 324-340 (again using a numbering system based on H3 HA) of many HA proteins found in natural influenza isolates. This sequence element has the basic structure:

GAIAGFIE

In some embodiments, HA Sequence Element 2 has the sequence:

PX₁GAIAGFIE, wherein:

X₁ is approximately 4-14 amino acids long, or about 8-12 amino acids long, or about 12, 11, 10, 9 or 8 amino acids long. In some embodiments, this sequence element provides the HA0 cleavage site, allowing production of HA1 and HA2.

In some embodiments, and particularly in H1 polypeptides, HA Sequence Element 2 has the structure:

PS(I/V)QSRX_{1A}GAIAGFIE, wherein:

X_{1A} is approximately 3 amino acids long; in some embodiments, X_{1A} is G (L/I) F.

In some embodiments, and particularly in H3 polypeptides, HA Sequence Element 2 has the structure:

PXKXTRX_{1A}GAIAGFIE, wherein:

X_{1A} is approximately 3 amino acids long; in some embodiments, X_{1A} is G (L/I) F.

In some embodiments, and particularly in H5 polypeptides, HA Sequence Element 2 has the structure:

PQRXXXRXXRX_{1A}GAIAGFIE, wherein:

X_{1A} is approximately 3 amino acids long; in some embodiments, X_{1A} is G (L/I) F.

### DEFINITIONS

Affinity: As is known in the art, “affinity” is a measure of the tightness with a particular ligand (e.g., an HA polypeptide) binds to its partner (e.g., and HA receptor). Affinities can be measured in different ways.

Biologically active: As used herein, the phrase “biologically active” refers to a characteristic of any agent that has activity in a biological system, and particularly in an organism. For instance, an agent that, when administered to an organism, has a biological effect on that organism, is considered to be biologically active. In particular embodiments, where a protein or polypeptide is biologically active, a portion of that protein or polypeptide that shares at least one biological activity of the protein or polypeptide is typically referred to as a “biologically active” portion.

Broad spectrum human-binding (BSHB) H5 HA polypeptides: As used herein, the phrase “broad spectrum human-binding H5 HA” refers to a version of an H5 HA polypeptide that binds to HA receptors found in human epithelial tissues, and particularly to human HA receptors having α2-6 sialylated glycans. Moreover, inventive BSHB H5 HAs bind to a plurality of different α2-6 sialylated glycans. In some embodiments, BSHB H5 HAs bind to a sufficient number of different α2-6 sialylated glycans found in human samples that viruses containing them have a broad ability to infect human populations, and particularly to bind to upper respiratory tract receptors in those populations. In some embodiments, BSHB H5 HA bind to umbrella glycans (e.g., long α2-6 sialylated glycans) as described herein.

Characteristic portion: As used herein, the phrase a “characteristic portion” of a protein or polypeptide is one that contains a continuous stretch of amino acids, or a collection of continuous stretches of amino acids, that together are characteristic of a protein or polypeptide. Each such continuous stretch generally will contain at least two amino acids. Furthermore, those of ordinary skill in the art will appreciate that typically at least 5, 10, 15, 20 or more amino acids are required to be characteristic of a protein. In general, a characteristic portion is one that, in addition to the sequence identity specified above, shares at least one functional characteristic with the relevant intact protein.

Characteristic sequence: A “characteristic sequence” is a sequence that is found in all members of a family of polypeptides or nucleic acids, and therefore can be used by those of ordinary skill in the art to define members of the family.

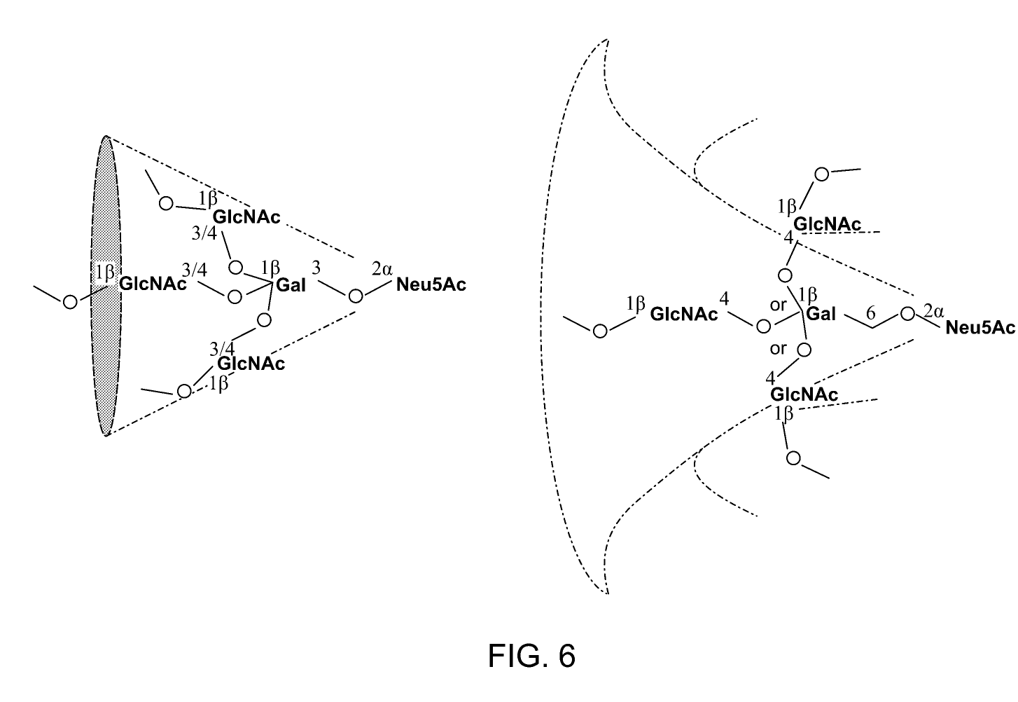

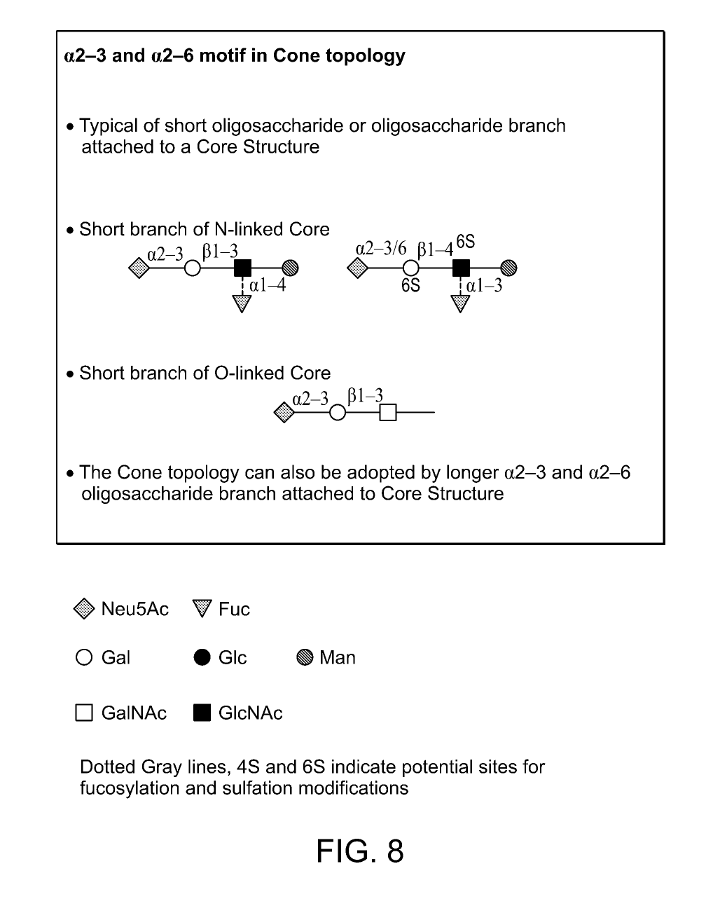

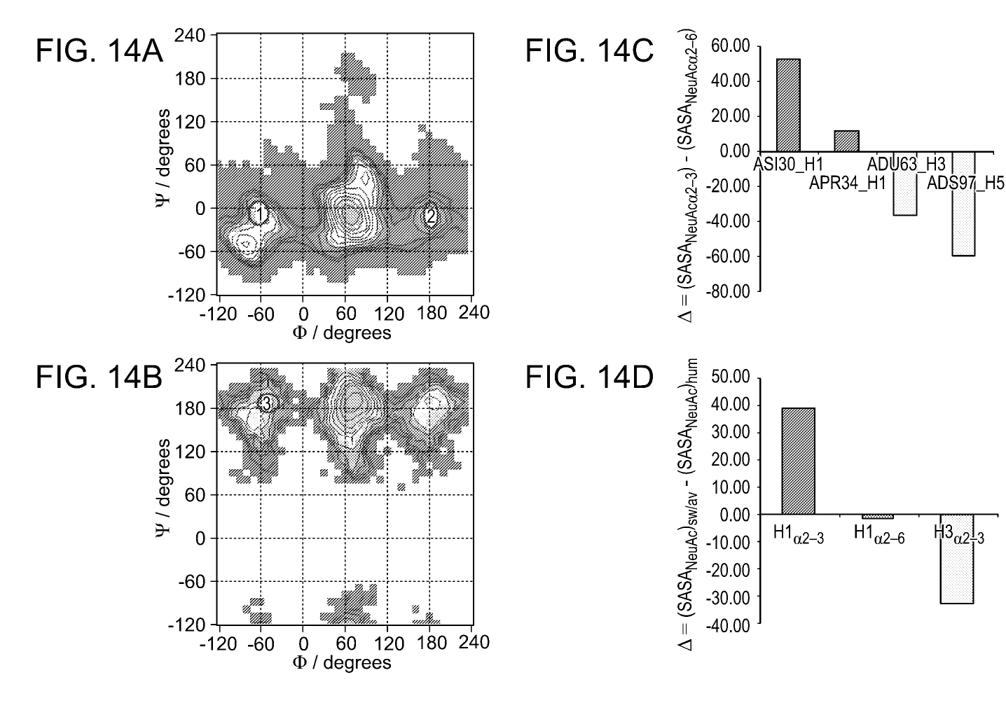

Cone topology: The phrase “cone topology” is used herein to refer to a 3-dimensional arrangement adopted by certain glycans and in particular by glycans on HA receptors. As illustrated in FIG. 6, the cone topology can be adopted by α2-3 sialylated glycans or by α2-6 sialylated glycans, and is typical of short oligonucleotide chains, though some long oligonucleotides can also adopt this conformation. The cone topology is characterized by the glycosidic torsion angles of Neu5Acα2-3Gal linkage which samples three regions of minimum energy conformations given by φ (C1-C2-O-C3/C6) value of around −60, 60 or 180 and ψ (C2-O-C3/C6-H3/C5) samples −60 to 60 (FIG. 14). FIG. 8 presents certain representative (though not exhaustive) examples of glycans that adopt a cone topology.

Corresponding to: As used herein, the term “corresponding to” is often used to designate the position/identity of an amino acid residue in an HA polypeptide. Those of ordinary skill will appreciate that, for purposes of simplicity, a canonical numbering system (based on wild type H3 HA) is utilized herein (as illustrated, for example, in FIGS. 1-5), so that an amino acid “corresponding to” a residue at position 190, for example, need not actually be the 190^{th} amino acid in a particular amino acid chain but rather corresponds to the residue found at 190 in wild type H3 HA; those of ordinary skill in the art readily appreciate how to identify corresponding amino acids.

Degree of separation removed: As used herein, amino acids that are a “degree of separation removed” are HA amino acids that have indirect effects on glycan binding. For example, one-degree-of-separation-removed amino acids may either: (1) interact with the direct-binding amino acids; and/or (2) otherwise affect the ability of direct-binding amino acids to interact with glycan that is associated with host cell HA receptors; such one-degree-of-separation-removed amino acids may or may not directly bind to glycan themselves. Two-degree-of-separation-removed amino acids either (1) interact with one-degree-of-separation-removed amino acids; and/or (2) otherwise affect the ability of the one-degree-of-separation-removed amino acids to interact with direct-binding amino acids, etc.

Direct-binding amino acids: As used herein, the phrase “direct-binding amino acids” refers to HA polypeptide amino acids which interact directly with one or more glycans that is associated with host cell HA receptors.

Engineered: The term “engineered”, as used herein, describes a polypeptide whose amino acid sequence has been selected by man. For example, an engineered HA polypeptide has an amino acid sequence that differs from the amino acid sequences of HA polypeptides found in natural influenza isolates. In some embodiments, an engineered HA polypeptide has an amino acid sequence that differs from the amino acid sequence of HA polypeptides included in the NCBI database.

H1 polypeptide: An “H1 polypeptide”, as that term is used herein, is an HA polypeptide whose amino acid sequence includes at least one sequence element that is characteristic of H1 and distinguishes H1 from other HA subtypes. Representative such sequence elements can be determined by alignments such as, for example, those illustrated in FIGS. 1-3 and include, for example, those described herein with regard to H1-specific embodiments of HA Sequence Elements.

H3 polypeptide: An “H3 polypeptide”, as that term is used herein, is an HA polypeptide whose amino acid sequence includes at least one sequence element that is characteristic of H3 and distinguishes H3 from other HA subtypes. Representative such sequence elements can be determined by alignments such as, for example, those illustrated in FIGS. 1, 2, and 4 and include, for example, those described herein with regard to H3-specific embodiments of HA Sequence Elements.

H5 polypeptide: An “H5 polypeptide”, as that term is used herein, is an HA polypeptide whose amino acid sequence includes at least one sequence element that is characteristic of H5 and distinguishes H5 from other HA subtypes. Representative such sequence elements can be determined by alignments such as, for example, those illustrated in FIGS. 1, 2, and 5 and include, for example, those described herein with regard to H5-specific embodiments of HA Sequence Elements.

Hemagglutinin (HA) polypeptide: As used herein, the term “hemagglutinin polypeptide” (or “HA polypeptide”) refers to a polypeptide whose amino acid sequence includes at least one characteristic sequence of HA. A wide variety of HA sequences from influenza isolates are known in the art; indeed, the National Center for Biotechnology Information (NCBI) maintains a database (www.ncbi.nlm.nih.gov/genomes/FLU/flu.html) that, as of the filing of the present application included 9796 HA sequences. Those of ordinary skill in the art, referring to this database, can readily identify sequences that are characteristic of HA polypeptides generally, and/or of particular HA polypeptides (e.g., H1, H2, H3, H4, H5, H6, H7, H8, H9, H10, H11, H12, H13, H14, H15, or H16 polypeptides; or of HAs that mediate infection of particular hosts, e.g., avian, camel, canine, cat, civet, environment, equine, human, leopard, mink, mouse, seal, stone martin, swine, tiger, whale, etc. For example, in some embodiments, an HA polypeptide includes one or more characteristic sequence elements found between about residues 97 and 185, 324 and 340, 96 and 100, and/or 130-230 of an HA protein found in a natural isolate of an influenza virus. In some embodiments, an HA polypeptide has an amino acid sequence comprising at least one of HA Sequence Elements 1 and 2, as defined herein. In some embodiments, an HA polypeptide has an amino acid sequence comprising HA Sequence Elements 1 and 2, in some embodiments separated from one another by about 100-200, or by about 125-175, or about 125-160, or about 125-150, or about 129-139, or about 129, 130, 131, 132, 133, 134, 135, 136, 137, 138, or 139 amino acids. In some embodiments, an HA polypeptide has an amino acid sequence that includes residues at positions within the regions 96-100 and/or 130-230 that participate in glycan binding. For example, many HA polypeptides include one or more of the following residues: Tyr98, Ser/Thr136, Trp153, His183, and Leu/Ile194. In some embodiments, an HA polypeptide includes at least 2, 3, 4, or all 5 of these residues.

Isolated: The term “isolated”, as used herein, refers to an agent or entity that has either (i) been separated from at least some of the components with which it was associated when initially produced (whether in nature or in an experimental setting); or (ii) produced by the hand of man. Isolated agents or entities may be separated from at least about 10%, 20%, 30%, 40%, 50%, 60%, 70%, 80%, 90%, or more of the other components with which they were initially associated. In some embodiments, isolated agents are more than 90%, 91%, 92%, 93%, 94%, 95%, 96%, 97%, 98%, 99% pure.

Long oligosaccharide: For purposes of the present disclosure, an oligosaccharide is typically considered to be “long” if it includes at least one linear chain that has at least four saccharide residues.

Non-natural amino acid: The phrase “non-natural amino acid” refers to an entity having the chemical structure of an amino acid (i.e.,:

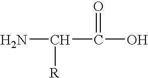

and therefore being capable of participating in at least two peptide bonds, but having an R group that differs from those found in nature. In some embodiments, non-natural amino acids may also have a second R group rather than a hydrogen, and/or may have one or more other substitutions on the amino or carboxylic acid moieties.

Polypeptide: A “polypeptide”, generally speaking, is a string of at least two amino acids attached to one another by a peptide bond. In some embodiments, a polypeptide may include at least 3-5 amino acids, each of which is attached to others by way of at least one peptide bond. Those of ordinary skill in the art will appreciate that polypeptides sometimes include “non-natural” amino acids or other entities that nonetheless are capable of integrating into a polypeptide chain, optionally.

Pure: As used herein, an agent or entity is “pure” if it is substantially free of other components. For example, a preparation that contains more than about 90% of a particular agent or entity is typically considered to be a pure preparation. In some embodiments, an agent or entity is at least 91%, 92%, 93%, 94%, 95%, 96%, 97%, 98%<Or 99% pure.

Short oligosaccharide: For purposes of the present disclosure, an oligosaccharide is typically considered to be “short” if it has fewer than 4, or certainly fewer than 3, residues in any linear chain.

Specificity: As is known in the art, “specificity” is a measure of the ability of a particular ligand (e.g., an HA polypeptide) to distinguish its binding partner (e.g., a human HA receptor, and particularly a human upper respiratory tract HA receptor) from other potential binding partners (e.g., an avian HA receptor).

Therapeutic agent: As used herein, the phrase “therapeutic agent” refers to any agent that elicits a desired biological or pharmacological effect.

Treatment: As used herein, the term “treatment” refers to any method used to alleviate, delay onset, reduce severity or incidence, or yield prophylaxis of one or more symptoms or aspects of a disease, disorder, or condition. For the purposes of the present invention, treatment can be administered before, during, and/or after the onset of symptoms.

Umbrella topology: The phrase “umbrella topology” is used herein to refer to a 3-dimensional arrangement adopted by certain glycans and in particular by glycans on HA receptors. The present invention encompasses the recognition that binding to umbrella topology glycans is characteristic of HA proteins that mediate infection of human hosts. As illustrated in FIG. 6, the umbrella topology is typically adopted only by α2-6 sialylated glycans, and is typical of long (e.g., greater than tetrasaccharide) oligosaccharides. An example of umbrella topology is given by φ angle of Neu5Acα2-6Gal linkage of around −60 (see, for example, FIG. 14). FIG. 9 presents certain representative (though not exhaustive) examples of glycans that adopt an umbrella topology.

Vaccination: As used herein, the term “vaccination” refers to the administration of a composition intended to generate an immune response, for example to a disease-causing agent. For the purposes of the present invention, vaccination can be administered before, during, and/or after exposure to a disease-causing agent, and in certain embodiments, before, during, and/or shortly after exposure to the agent. In some embodiments, vaccination includes multiple administrations, appropriately spaced in time, of a vaccinating composition.

Variant: As used herein, the term “variant” is a relative term that describes the relationship between a particular HA polypeptide of interest and a “parent” HA polypeptide to which its sequence is being compared. An HA polypeptide of interest is considered to be a “variant” of a parent HA polypeptide if the HA polypeptide of interest has an amino acid sequence that is identical to that of the parent but for a small number of sequence alterations at particular positions. Typically, fewer than 20%, 15%, 10%, 9%, 8%, 7%, 6%, 5%, 4%, 3%, 2% of the residues in the variant are substituted as compared with the parent. In some embodiments, a variant has 10, 9, 8, 7, 6, 5, 4, 3, 2, or 1 substituted residue as compared with a parent. Often, a variant has a very small number (e.g., fewer than 5, 4, 3, 2, or 1) number of substituted functional residues (i.e., residues that participate in a particular biological activity). Furthermore, a variant typically has not more than 5, 4, 3, 2, or 1 additions or deletions, and often has no additions or deletions, as compared with the parent. Moreover, any additions or deletions are typically fewer than about 25, 20, 19, 181, 17, 16, 15, 14, 13, 10, 9, 8, 7, 6, and commonly are fewer than about 5, 4, 3, or 2 residues. In some embodiments, the parent HA polypeptide is one found in a natural isolate of an influenza virus (e.g., a wild type HA).

Vector: As used herein, “vector” refers to a nucleic acid molecule capable of transporting another nucleic acid to which it has been linked. In some embodiment, vectors are capable of extra-chromosomal replication and/or expression of nucleic acids to which they are linked in a host cell such as a eukaryotic or prokaryotic cell. Vectors capable of directing the expression of operatively linked genes are referred to herein as “expression vectors.”

Wild type: As is understood in the art, the phrase “wild type” generally refers to a normal form of a protein or nucleic acid, as is found in nature. For example, wild type HA polypeptides are found in natural isolates of influenza virus. A variety of different wild type HA sequences can be found in the NCBI influenza virus sequence database, http://www.ncbi.nlm.nih.gov/genomes/FLU/FLU.html.

### DETAILED DESCRIPTION OF CERTAIN PARTICULAR EMBODIMENTS OF THE INVENTION

The present invention provides HA polypeptides that bind to umbrella topology glycans. In some embodiments, the present invention provides HA polypeptides that bind to umbrella topology glycans found on HA receptors of a particular target species. For example, in some embodiments, the present invention provides HA polypeptides that bind to umbrella topology glycans found on human HA receptors, e.g., HA receptors found on human epithelial cells, and particularly HA polypeptides that bind to umbrella topology glycans found on human HA receptors in the upper respiratory tract.

The present invention provides HA polypeptides that bind to HA receptors found on cells in the human upper respiratory tract, and in particular provides HA polypeptides that binds to such receptors (and/or to their glycans, particularly to their umbrella glycans) with a designated affinity and/or specificity.

The present invention encompasses the recognition that gaining an ability to bind umbrella topology glycans (e.g., long a2-6 sialylated glycans), and particularly an ability to bind with high affinity, may confer upon an HA polypeptide variant the ability to infect humans (where its parent HA polypeptide cannot). Without wishing to be bound by any particular theory, the present inventors propose that binding to umbrella topology glycans may be paramount, and in particular that loss of binding to other glycan types may not be required.

The present invention further provides various reagents and methods associated with inventive HA polypeptides including, for example, systems for identifying them, strategies for preparing them, antibodies that bind to them, and various diagnostic and therapeutic methods relating to them. Further description of certain embodiments of these aspects, and others, of the present invention, is presented below.

### Hemagglutinin (HA)

Influenza viruses are RNA viruses which are characterized by a lipid membrane envelope containing two glycoproteins, hemagglutinin (HA) and neuraminidase (NA), embedded in the membrane of the virus particular. There are 16 known HA subtypes and 9 NA subtypes, and different influenza strains are named based on the number of the strain's HA and NA subtypes. Based on comparisons of amino acid sequence identity and of crystal structures, the HA subtypes have been divided into two main groups and four smaller clades. The different HA subtypes do not necessarily share strong amino acid sequence identity, but the overall 3D structures of the different HA subtypes are similar to one another, with several subtle differences that can be used for classification purposes. For example, the particular orientation of the membrane-distal subdomains in relation to a central α-helix is one structural characteristic commonly used to determine HA subtype (Russell et al., Virology, 325:287, 2004).

HA exists in the membrane as a homotrimer of one of 16 subtypes, termed H1-H16. Only three of these subtypes (H1, H2, and H3) have thus far become adapted for human infection. One reported characteristic of HAs that have adapted to infect humans (e.g., of HAs from the pandemic H1N1 (1918) and H3N2 (1967-68) influenza subtypes) is their ability to preferentially bind to α2-6 sialylated glycans in comparison with their avian progenitors that preferentially bind to α2-3 sialylated glycans (Skehel & Wiley, Annu Rev Biochem, 69:531, 2000; Rogers, & Paulson, Virology, 127:361, 1983; Rogers et al., Nature, 304:76, 1983; Sauter et al., Biochemistry, 31:9609, 1992; Connor et al., Virology, 205:17, 1994; Tumpey et al., Science, 310:77, 2005). The present invention, however, encompasses the recognition that ability to infect human hosts correlates less with binding to glycans of a particular linkage, and more with binding to glycans of a particular topology. Thus, the present invention demonstrates that HAs that mediate infection of humans bind to umbrella topology glycans, often showing preference for umbrella topology glycans over cone topology glycans (even though cone-topology glycans may be α2-6 sialylated glycans).

Several crystal structures of HAs from H1 (human and swine), H3 (avian) and H5 (avian) subtypes bound to sialylated oligosaccharides (of both α2-3 and α2-6 linkages) are available and provide molecular insights into the specific amino acids that are involved in distinct interactions of the HAs with these glycans (Eisen et al., Virology, 232:19, 1997; Ha et al., Proc Natl Acad Sci USA, 98:11181, 2001; Ha et al., Virology, 309:209, 2003; Gamblin et al., Science, 303:1838, 2004; Stevens et al., Science, 303:1866, 2004; Russell et al., Glycoconj J 23:85, 2006; Stevens et al., Science, 312:404, 2006).

For example, the crystal structures of H5 (A/duck/Singapore/3/97) alone or bound to an α2-3 or an α2-6 sialylated oligosaccharide identifies certain amino acids that interact directly with bound glycans, and also amino acids that are one or more degree of separation removed (Stevens et al., Proc Natl Acad Sci USA 98:11181, 2001). In some cases, conformation of these residues is different in bound versus unbound states. For instance, Glu190, Lys193 and Gln226 all participate in direct-binding interactions and have different conformations in the bound versus the unbound state. The conformation of Asn186, which is proximal to Glu190, is also significantly different in the bound versus the unbound state.

### Binding Characteristics of Inventive HA Polypeptides

As noted above, the present invention encompasses the finding that binding to umbrella topology glycans correlates with ability to mediate infection of particular hosts, including for example, humans. Accordingly, the present invention provides HA polypeptides that bind to umbrella glycans. In certain embodiments, inventive HA polypeptides bind to umbrella glycans with high affinity. In certain embodiments, inventive HA polypeptides bind to a plurality of different umbrella topology glycans, often with high affinity and/or specificity.

In some embodiments, inventive HA polypeptides bind to umbrella topology glycans (e.g., long α2-6 silaylated glycans such as, for example, Neu5Acα2-6Galβ1-4GlcNAcβ1-3Galβ1-4GlcNAc-) with high affinity. For example, in some embodiments, inventive HA polypeptides bind to umbrella topology glycans with an affinity comparable to that observed for a wild type HA that mediates infection of a humans (e.g., H1N1 HA or H3N2 HA). In some embodiments, inventive HA polypeptides bind to umbrella glycans with an affinity that is at least 25%, 30%, 35%, 40%, 45%, 50%, 55%, 60%, 65%, 70%, 75%, 80%, 85%, 90%, 95%, 96%, 97%, 98%, 99%, or 100% of that observed under comparable conditions for a wild type HA that mediates infection of humans. In some embodiments, inventive HA polypeptides bind to umbrella glycans with an affinity that is greater than that observed under comparable conditions for a wild type HA that mediates infection of humans.

In certain embodiments, binding affinity of inventive HA polypeptides is assessed over a range of concentrations. Such a strategy provides significantly more information, particularly in multivalent binding assays, than do single-concentration analyses. In some embodiments, for example, binding affinities of inventive HA polypeptides are assessed over concentrations ranging over at least 2, 3, 4, 5, 6, 7, 8, 9, 10 or more fold.

In certain embodiments, inventive HA polypeptides show high affinity if they show a saturating signal in a multivalent glycan array binding assay such as those described herein. In some embodiments, inventive HA polypeptides show high affinity if they show a signal above about 400000 or more (e.g., above about 500000, 600000, 700000, 800000, etc) in such studies. In some embodiments, HA polypeptides show saturating binding to umbrella glycans over a concentration range of at least 2 fold, 3 fold, 4 fold, 5 fold or more, and in some embodiments over a concentration range as large as 10 fold or more.

Furthermore, in some embodiments, inventive HA polypeptides bind to umbrella topology glycans more strongly than they bind to cone topology glycans. In some embodiments, inventive HA polypeptides show a relative affinity for umbrella glycans vs cone glycans that is about 10, 9, 8, 7, 6, 5, 4, 3, or 2.

In some embodiments, inventive HA polypeptides bind to α2-6 sialylated glycans; in some embodiments, inventive HA polypeptides bind preferentially to α2-6 sialylated glycans. In certain embodiments, inventive HA polypeptides bind to a plurality of different α2-6 sialylated glycans. In some embodiments, inventive HA polypeptides are not able to bind to α2-3 sialylated glycans, and in other embodiments inventive HA polypeptides are able to bind to α2-3 sialylated glycans.

In some embodiments, inventive HA polypeptides bind to receptors found on human upper respiratory epithelial cells. In certain embodiments, inventive HA polypeptides bind to HA receptors in the bronchus and/or trachea. In some embodiments, inventive HA polypeptides are not able to bind receptors in the deep lung, and in other embodiments, inventive HA polypeptides are able to bind receptors in the deep lung.

In some embodiments, inventive HA polypeptides bind to at least about 10%, 15%, 20%, 25%, 30% 35%, 40%, 45%, 50%, 55%, 60%, 65%, 70%, 75%, 80%, 85%, 90% 95% or more of the glycans found on HA receptors in human upper respiratory tract tissues (e.g., epithelial cells).

In some embodiments, inventive HA polypeptides bind to one or more of the glycans illustrated in FIG. 9. In some embodiments, inventive HA polypeptides bind to multiple glycans illustrated in FIG. 9. In some embodiments, inventive HA polypeptides bind with high affinity and/or specificity to glycans illustrated in FIG. 9. In some embodiments, inventive HA polypeptides bind to glycans illustrated in FIG. 9 preferentially as compared with their binding to glycans illustrated in FIG. 8.

The present invention provides isolated HA polypeptides with designated binding specificity, and also provides engineered HA polypeptides with designated binding characteristics with respect to umbrella glycans.

In some embodiments, inventive HA polypeptides with designated binding characteristics are H1 polypeptides. In some embodiments, inventive HA polypeptides with designated binding characteristics are H2 polypeptides. In some embodiments, inventive HA polypeptides with designated binding characteristics are H3 polypeptides. In some embodiments, inventive HA polypeptides with designated binding characteristics are H4 polypeptides. In some embodiments, inventive HA polypeptides with designated binding characteristics are H5 polypeptides. In some embodiments, inventive HA polypeptides with designated binding characteristics are H6 polypeptides. In some embodiments, inventive HA polypeptides with designated binding characteristics are H7 polypeptides. In some embodiments, inventive HA polypeptides with designated binding characteristics are H8 polypeptides. In some embodiments, inventive HA polypeptides with designated binding characteristics are H9 polypeptides. In some embodiments, inventive HA polypeptides with designated binding characteristics are H10 polypeptides. In some embodiments, inventive HA polypeptides with designated binding characteristics are H11 polypeptides. In some embodiments, inventive HA polypeptides with designated binding characteristics are H12 polypeptides. In some embodiments, inventive HA polypeptides with designated binding characteristics are H13 polypeptides. In some embodiments, inventive HA polypeptides with designated binding characteristics are H14 polypeptides. In some embodiments, inventive HA polypeptides with designated binding characteristics are H15 polypeptides. In some embodiments, inventive HA polypeptides with designated binding characteristics are H16 polypeptides.

In some embodiments, inventive HA polypeptides with designated binding characteristics are not H1 polypeptides, are not H2 polypeptides, and/or are not H3 polypeptides.

In some embodiments, inventive HA polypeptides do not include the H1 protein from any of the strains: A/South Carolina/1/1918; A/Puerto Rico/8/1934; A/Taiwan/1/1986; A/Texas/36/1991; A/Beijing/262/1995; A/Johannesburg/92/1996; A/New Caledonia/20/1999; A/Solomon Islands/3/2006.

In some embodiments, inventive HA polypeptides are not the H2 protein from any of the strains of the Asian flu epidemic of 1957-58). In some embodiments, inventive HA polypeptides do not include the H2 protein from any of the strains: A/Japan/305+/1957; A/Singapore/1/1957; A/Taiwan/1/1964; A/Taiwan/1/1967.

In some embodiments, inventive HA polypeptides do not include the H3 protein from any of the strains: A/Aichi/2/1968; A/Phillipines/2/1982; A/Mississippi/1/1985; A/Leningrad/360/1986; A/Sichuan/2/1987; A/Shanghai/11/1987; A/Beijing/353/1989; A/Shandong/9/1993; A/Johannesburg/33/1994; A/Nanchang/813/1995; A/Sydney/5/1997; A/Moscow/10/1999; A/Panama/2007/1999; A/Wyoming/3/2003; A/Oklahoma/323/2003; A/California/7/2004; A/Wisconsin/65/2005.

### Variant HA Polypeptides

In certain embodiments, an HA polypeptide is a variant of a parent HA polypeptide in that its amino acid sequence is identical to that of the parent HA but for a small number of particular sequence alterations. In some embodiments, the parent HA is an HA polypeptide found in a natural isolate of an influenza virus (e.g., a wild type HA polypeptide).

In some embodiments, inventive HA polypeptide variants have different glycan binding characteristics than their corresponding parent HA polypeptides. In some embodiments, inventive HA variant polypeptides have greater affinity and/or specificity for umbrella glycans (e.g., as compared with for cone glycans) than do their cognate parent HA polypeptides. In certain embodiments, such HA polypeptide variants are engineered variants.

In some embodiments, HA polypeptide variants with altered glycan binding characteristics have sequence alternations in residues within or affecting the glycan binding site. In some embodiments, such substitutions are of amino acids that interact directly with bound glycan; in other embodiments, such substitutions are of amino acids that are one degree of separation removed from those that interact with bound glycan, in that the one degree of separation removed-amino acids either (1) interact with the direct-binding amino acids; (2) otherwise affect the ability of the direct-binding amino acids to interact with glycan, but do not interact directly with glycan themselves; or (3) otherwise affect the ability of the direct-binding amino acids to interact with glycan, and also interact directly with glycan themselves. Inventive HA polypeptide variants contain substitutions of one or more direct-binding amino acids, one or more first degree of separation-amino acids, one or more second degree of separation-amino acids, or any combination of these. In some embodiments, inventive HA polypeptide variants may contain substitutions of one or more amino acids with even higher degrees of separation.

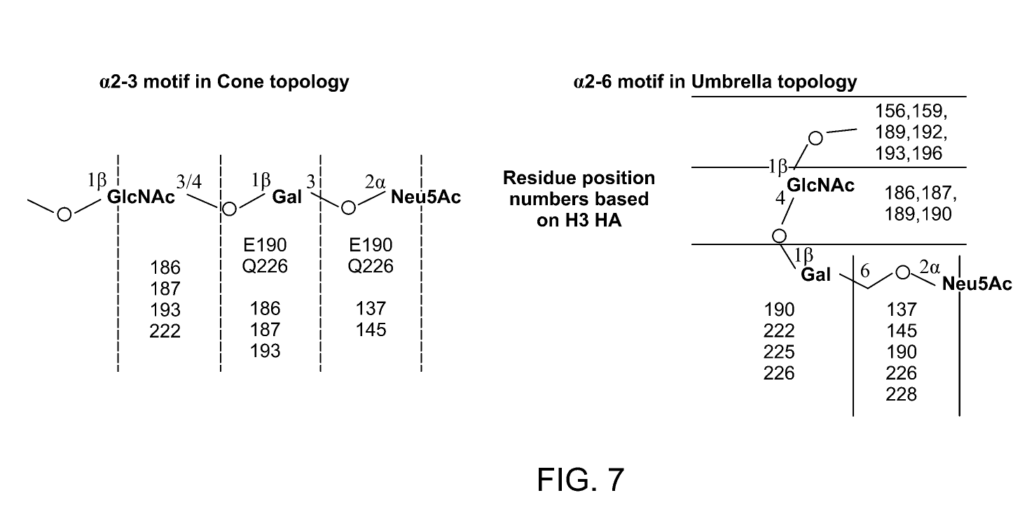

In some embodiments, HA polypeptide variants with altered glycan binding characteristics have sequence alterations in residues that make contact with sugars beyond Neu5Ac and Gal (see, for example, FIG. 7).

In some embodiments, HA polypeptide variants have at least one amino acid substitution, as compared with a wild type parent HA. In certain embodiments, inventive HA polypeptide variants have at least two, three, four, five or more amino acid substitutions as compared with a cognate wild type parent HA; in some embodiments inventive HA polypeptide variants have two, three, or four amino acid substitutions. In some embodiments, all such amino acid substitutions are located within the glycan binding site.

In some embodiments, HA polypeptide variants have sequence substitutions at positions corresponding to one or more of residues 137, 145, 156, 159, 186, 187, 189, 190, 192, 193, 196, 222, 225, 226, and 228. In some embodiments, HA polypeptide variants have sequence substitutions at positions corresponding to one or more of residues 156, 159, 189, 192, 193, and 196; and/or at positions corresponding to one or more of residues 186, 187, 189, and 190; and/or at positions corresponding to one or more of residues 190, 222, 225, and 226; and/or at positions corresponding to one or more of residues 137, 145, 190, 226 and 228. In some embodiments, HA polypeptide variants have sequence substitutions at positions corresponding to one or more of residues 190, 225, 226, and 228.

In certain embodiments, HA polypeptide variants, and particularly H5 polypeptide variants, have one or more amino acid substitutions relative to a wild type parent HA (e.g., H5) at residues selected from the group consisting of residues 98, 136, 138, 153, 155, 159, 183, 186, 187, 190, 193, 194, 195, 222, 225, 226, 227, and 228. In other embodiments, HA polypeptide variants, and particularly H5 polypeptide variants, have one or more amino acid substitutions relative to a wild type parent HA at residues selected from amino acids located in the region of the receptor that directly binds to the glycan, including but not limited to residues 98, 136, 153, 155, 183, 190, 193, 194, 222, 225, 226, 227, and 228. In further embodiments, an HA polypeptide variant, and particularly an H5 polypeptide variant, has one or more amino acid substitutions relative to a wild type parent HA at residues selected from amino acids located adjacent to the region of the receptor that directly binds the glycan, including but not limited to residues 98, 138, 186, 187, 195, and 228.

In some embodiments, an inventive HA polypeptide variant, and particularly an H5 polypeptide variant has one or more amino acid substitutions relative to a wild type parent HA at residues selected from the group consisting of residues 138, 186, 187, 190, 193, 222, 225, 226, 227 and 228. In other embodiments, an inventive HA polypeptide variant, and particularly an H5 polypeptide variant, has one or more amino acid substitutions relative to a wild type parent HA at residues selected from amino acids located in the region of the receptor that directly binds to the glycan, including but not limited to residues 190, 193, 222, 225, 226, 227, and 228. In further embodiments, an inventive HA polypeptide variant, and particularly an H5 polypeptide variant, has one or more amino acid substitutions relative to a wild type parent HA at residues selected from amino acids located adjacent to the region of the receptor that directly binds the glycan, including but not limited to residues 138, 186, 187, and 228.

In further embodiments, an HA polypeptide variant, and particularly an H5 polypeptide variant, has one or more amino acid substitutions relative to a wild type parent HA at residues selected from the group consisting of residues 98, 136, 153, 155, 183, 194, and 195. In other embodiments, an HA polypeptide variant, and particularly an H5 polypeptide variant, has one or more amino acid substitutions relative to a wild type parent HA at residues selected from amino acids located in the region of the receptor that directly binds to the glycan, including but not limited to residues 98, 136, 153, 155, 183, and 194. In further embodiments, an inventive HA polypeptide variant, and particularly an H5 polypeptide variant, has one or more amino acid substitutions relative to a wild type parent HA at residues selected from amino acids located adjacent to the region of the receptor that directly binds the glycan, including but not limited to residues 98 and 195.

In certain embodiments, an HA polypeptide variant, and particularly an H5 polypeptide variant has one or more amino acid substitutions relative to a wild type parent HA at residues selected from amino acids that are one degree of separation removed from those that interact with bound glycan, in that the one degree of separation removed-amino acids either (1) interact with the direct-binding amino acids; (2) otherwise affect the ability of the direct-binding amino acids to interact with glycan, but do not interact directly with glycan themselves; or (3) otherwise affect the ability of the direct-binding amino acids to interact with glycan, and also interact directly with glycan themselves, including but not limited to residues 98, 138, 186, 187, 195, and 228.

In further embodiments, an HA polypeptide variant, and particularly an H5 polypeptide variant, has one or more amino acid substitutions relative to a wild type parent HA at residues selected from amino acids that are one degree of separation removed from those that interact with bound glycan, in that the one degree of separation removed-amino acids either (1) interact with the direct-binding amino acids; (2) otherwise affect the ability of the direct-binding amino acids to interact with glycan, but do not interact directly with glycan themselves; or (3) otherwise affect the ability of the direct-binding amino acids to interact with glycan, and also interact directly with glycan themselves, including but not limited to residues 138, 186, 187, and 228.

In further embodiments, an HA polypeptide variant, and particularly an H5 polypeptide variant, has one or more amino acid substitutions relative to a wild type parent HA at residues selected from amino acids that are one degree of separation removed from those that interact with bound glycan, in that the one degree of separation removed-amino acids either (1) interact with the direct-binding amino acids; (2) otherwise affect the ability of the direct-binding amino acids to interact with glycan, but do not interact directly with glycan themselves; or (3) otherwise affect the ability of the direct-binding amino acids to interact with glycan, and also interact directly with glycan themselves, including but not limited to residues 98 and 195.

In certain embodiments, an HA polypeptide variant, and particularly an H5 polypeptide variant, has an amino acid substitution relative to a wild type parent HA at residue 159.

In other embodiments, an HA polypeptide variant, and particularly an H5 polypeptide variant, has one or more amino acid substitutions relative to a wild type parent HA at residues selected from 190, 193, 225, and 226. In some embodiments, an HA polypeptide variant, and particularly an H5 polypeptide variant, has one or more amino acid substitutions relative to a wild type parent HA at residues selected from 190, 193, 226, and 228.

In some embodiments, an inventive HA polypeptide variant, and particularly an H5 variant has one or more of the following amino acid substitutions: Ser137Ala, Lys156Glu, Asn186Pro, Asp187Ser, Asp187Thr, Ala189Gln, Ala189Lys, Ala189Thr, Glu190Asp, Glu190Thr, Lys193Arg, Lys193Asn, Lys193His, Lys193Ser, Gly225Asp, Gln226Ile, Gln226Leu, Gln226Val, Ser227Ala, Gly228Ser.

In some embodiments, an inventive HA polypeptide variant, and particularly an H5 variant has one or more of the following sets of amino acid substitutions:

Glu190Asp, Lys193Ser, Gly225Asp and Gln226Leu;

Glu190Asp, Lys193Ser, Gln226Leu and Gly228Ser;

Ala189Gln, Lys193Ser, Gln226Leu, Gly228Ser;

Ala189Gln, Lys193Ser, Gln226Leu, Gly228Ser;

Asp187Ser/Thr, Ala189Gln, Lys193Ser, Gln226Leu, Gly228Ser;

Ala189Lys, Lys193Asn, Gln226Leu, Gly228Ser;

Asp187Ser/Thr, Ala189Lys, Lys193Asn, Gln226Leu, Gly228Ser;

Lys156Glu, Ala189Lys, Lys193Asn, Gln226Leu, Gly228Ser;

Lys193His, Gln226Leu/Ile/Val, Gly228Ser;

Lys193Arg, Gln226Leu/Ile/Val, Gly228Ser;

Ala189Lys, Lys193Asn, Gly225Asp;

Lys156Glu, Ala189Lys, Lys193Asn, Gly225Asp;

Ser137Ala, Lys156Glu, Ala189Lys, Lys193Asn, Gly225Asp;

Glu190Thr, Lys193Ser, Gly225Asp;

Asp187Thr, Ala189Thr, Glu190Asp, Lys193Ser, Gly225Asp;

Asn186Pro, Asp187Thr, Ala189Thr, Glu190Asp, Lys193Ser, Gly225Asp;

Asn186Pro, Asp187Thr, Ala189Thr, Glu190Asp, Lys193Ser, Gly225Asp, Ser227Ala. In some such embodiments, the HA polypeptide has at least one further substitution as compared with a wild type HA, such that affinity and/or specificity of the variant for umbrella glycans is increased.

In some embodiments, inventive HA polypeptides (including HA polypeptide variants) have sequences that include D190, D225, L226, and/or S228. In some embodiments, inventive HA polypeptides have sequences that include D190 and D225; in some embodiments, inventive HA polypeptides have sequences that include L226 and S228.

In some embodiments, inventive HA polypeptide variants have an open binding site as compared with a parent HA, and particularly with a parent wild type HAs.

### Portions or Fragments of HA Polypeptides

The present invention further provides characteristic portions of inventive HA polypeptides and nucleic acids that encode them. In general, a characteristic portion is one that contains a continuous stretch of amino acids, or a collection of continuous stretches of amino acids, that together are characteristic of the HA polypeptide. Each such continuous stretch generally will contain at least two amino acids. Furthermore, those of ordinary skill in the art will appreciate that typically at least 5, 10, 15, 20 or more amino acids are required to be characteristic of a H5 HA polypeptide. In general, a characteristic portion is one that, in addition to the sequence identity specified above, shares at least one functional characteristic with the relevant intact HA polypeptide. In some embodiments, inventive characteristic portions of HA polypeptides share glycan binding characteristics with the relevant full-length HA polypeptides.

### Production of HA Polypeptides

Inventive HA polypeptides, and/or characteristic portions thereof, or nucleic acids encoding them, may be produced by any available means.

Inventive HA polypeptides (or characteristic portions) may be produced, for example, by utilizing a host cell system engineered to express an inventive HA-polypeptide-encoding nucleic acid.

Any system can be used to produce HA polypeptides (or characteristic portions), such as egg, baculovirus, plant, yeast, Madin-Darby Canine Kidney cells (MDCK), or Vero (African green monkey kidney) cells. Alternatively or additionally, HA polypeptides (or characteristic portions) can be expressed in cells using recombinant techniques, such as through the use of an expression vector (Sambrook et al., Molecular Cloning: A Laboratory Manual, CSHL Press, 1989).

Alternatively or additionally, inventive HA polypeptides (or characteristic portions thereof) can be produced by synthetic means.

Alternatively or additionally, inventive HA polypeptides (or characteristic portions thereof) may be produced in the context of intact virus, whether otherwise wild type, attenuated, killed, etc. Inventive HA polypeptides, or characteristic portions thereof, may also be produced in the context of virus like particles.

In some embodiments, HA polypeptides (or characteristic portions thereof) can be isolated and/or purified from influenza virus. For example, virus may be grown in eggs, such as embryonated hen eggs, in which case the harvested material is typically allantoic fluid. Alternatively or additionally, influenza virus may be derived from any method using tissue culture to grow the virus. Suitable cell substrates for growing the virus include, for example, dog kidney cells such as MDCK or cells from a clone of MDCK, MDCK-like cells, monkey kidney cells such as AGMK cells including Vero cells, cultured epithelial cells as continuous cell lines, 293T cells, BK-21 cells, CV-1 cells, or any other mammalian cell type suitable for the production of influenza virus for vaccine purposes, readily available from commercial sources (e.g., ATCC, Rockville, Md.). Suitable cell substrates also include human cells such as MRC-5 cells. Suitable cell substrates are not limited to cell lines; for example primary cells such as chicken embryo fibroblasts are also included.

Also, it will be appreciated by those of ordinary skill in the art that HA polypeptides, and particularly variant HA polypeptides as described herein, may be generated, identified, isolated, and/or produced by culturing cells or organisms that produce the HA (whether alone or as part of a complex, including as part of a virus particle or virus), under conditions that allow ready screening and/or selection of HA polypeptides capable of binding to umbrella-topology glycans. To give but one example, in some embodiments, it may be useful to produce and/or study a collection of HA variants under conditions that reveal and/or favor those variants that bind to umbrella topology glycans (e.g., with particular specificity and/or affinity). In some embodiments, such a collection of HA variants results from evolution in nature. In some embodiments, such a collection of HA variants results from engineering. In some embodiments, such a collection of HA variants results from a combination of engineering and natural evolution.

### HA Receptors

HA interacts with the surface of cells by binding to a glycoprotein receptor. Binding of HA to HA receptors is predominantly mediated by N-linked glycans on the HA receptors. Specifically, HA on the surface of flu virus particles recognizes sialylated glycans that are associated with HA receptors on the surface of the cellular host. After recognition and binding, the host cell engulfs the viral cell and the virus is able to replicate and produce many more virus particles to be distributed to neighboring cells.

HA receptors are modified by either α2-3 or α2-6 sialylated glycans near the receptor's HA-binding site, and the type of linkage of the receptor-bound glycan affects the conformation of the receptor's HA-binding site, thus affecting the receptor's specificity for different HAs.

For example, the glycan binding pocket of avian HA is narrow. According to the present invention, this pocket binds to the trans conformation of α2-3 sialylated glycans, and/or to cone-topology glycans, whether α2-3 or α2-6 linked.

HA receptors in avian tissues, and also in human deep lung and gastrointestinal (GI) tract tissues are characterized by α2-3 sialylated glycan linkages, and furthermore (according to the present invention), are characterized by glycans, including α2-3 sialylated and/or α2-6 sialylated glycans, which predominantly adopt cone topologies.

By contrast, human HA receptors in the bronchus and trachea of the upper respiratory tract are modified by α2-6 sialylated glycans. Unlike the α2-3 motif, the α2-6 motif has an additional degree of conformational freedom due to the C6-C5 bond (Russell et al., Glycoconj J 23:85, 2006). HAs that bind to such α2-6 sialylated glycans have a more open binding pocket to accommodate the diversity of structures arising from this conformational freedom. Moreover, according to the present invention, HAs may need to bind to glycans (e.g., α2-6 sialylated glycans) in an umbrella topology, and particularly may need to bind to such umbrella topology glycans with strong affinity and/or specificity, in order to effectively mediate infection of human upper respiratory tract tissues.

As a result of these spatially restricted glycosylation profiles, humans are not usually infected by viruses containing many wild type avian HAs (e.g., avian H5). Specifically, because the portions of the human respiratory tract that are most likely to encounter virus (i.e., the trachea and bronchi) lack receptors with cone glycans (e.g., α2-3 sialylated glycans, and/or short glycans) and wild type avian HAs typically bind primarily or exclusively to receptors associated with cone glycans (e.g., α2-3 sialylated glycans, and/or short glycans), humans rarely become infected with avian viruses. Only when in sufficiently close contact with virus that it can access the deep lung and/or gastrointestinal tract receptors having umbrella glycans (e.g., long α2-6 sialylated glycans) do humans become infected.

### Glycan Arrays

To rapidly expand the current knowledge of known specific glycan-glycan binding protein (GBP) interactions, the Consortium for Functional Glycomics (CFG; www.functionalglycomics.org), an international collaborative research initiative, has developed glycan arrays comprising several glycan structures that have enabled high throughput screening of GBPs for novel glycan ligand specificities. The glycan arrays comprise both monovalent and polyvalent glycan motifs (i.e. attached to polyacrylamide backbone), and each array comprises 264 glycans with low (10 uM) and high (100 uM) concentrations, and six spots for each concentration (see http://www.functionalglycomics.org/static/consortium/resources/resourcecoreh5.shtml).

The arrays predominantly comprise synthetic glycans that capture the physiological diversity of N- and O-linked glycans. In addition to the synthetic glycans, N-linked glycan mixtures derived from different mammalian glycoproteins are also represented on the array.

As used herein, a glycan “array” refers to a set of one or more glycans, optionally immobilized on a solid support. In some embodiments, an “array” is a collection of glycans present as an organized arrangement or pattern at two or more locations that are physically separated in space. Typically, a glycan array will have at least 4, 8, 16, 24, 48, 96 or several hundred or thousand discrete locations. In general, inventive glycan arrays may have any of a variety of formats. Various different array formats applicable to biomolecules are known in the art. For example, a huge number of protein and/or nucleic acid arrays are well known. Those of ordinary skill in the art will immediately appreciate standard array formats appropriate for glycan arrays of the present invention.

In some embodiments, inventive glycan arrays are present in “microarray” formats. A microarray may typically have sample locations separated by a distance of 50-200 microns or less and immobilized sample in the nano to micromolar range or nano to picogram range. Array formats known in the art include, for example, those in which each discrete sample location has a scale of, for example, ten microns.

In some embodiments, inventive glycan arrays comprise a plurality of glycans spatially immobilized on a support. The present invention provides glycan molecules arrayed on a support. As used herein, “support” refers to any material which is suitable to be used to array glycan molecules. As will be appreciated by those of ordinary skill in the art, any of a wide variety of materials may be employed. To give but a few examples, support materials which may be of use in the invention include hydrophobic membranes, for example, nitrocellulose, PVDF or nylon membranes. Such membranes are well known in the art and can be obtained from, for example, Bio-Rad, Hemel Hempstead, UK.

In further embodiments, the support on which glycans are arrayed may comprise a metal oxide. Suitable metal oxides include, but are not limited to, titanium oxide, tantalum oxide, and aluminium oxide. Examples of such materials may be obtained from Sigma-Aldrich Company Ltd, Fancy Road, Poole, Dorset. BH12 4QH UK.

In yet further embodiments, such a support is or comprises a metal oxide gel. A metal oxide gel is considered to provide a large surface area within a given macroscopic area to aid immobilization of the carbohydrate-containing molecules.

Additional or alternative support materials which may be used in accordance with the present invention include gels, for example silica gels or aluminum oxide gels. Examples of such materials may be obtained from, for example, Merck KGaA, Darmstadt, Germany.

In some embodiments of the invention, glycan arrays are immobilized on a support that can resist change in size or shape during normal use. For example a support may be a glass slide coated with a component material suitable to be used to array glycans. Also, some composite materials can desirable provide solidity to a support.

As demonstrated herein, inventive arrays are useful for the identification and/or characterization of different HA polypeptides and their binding characteristics. In certain embodiments, inventive HA polypeptides are tested on such arrays to assess their ability to bind to umbrella topology glycans (e.g., to α2-6 sialylated glycans, and particularly to long α2-6 sialylated glycans arranged in an umbrella topology).

Indeed, the present invention provides arrays of α2-6 sialylated glycans, and optionally α2-3 sialylated glycans, that can be used to characterize HA polypeptide binding capabilities and/or as a diagnostic to detect, for example, human-binding HA polypeptides. In some embodiments, inventive arrays contain glycans (e.g., α2-6 sialylated glycans, and particularly long α2-6 sialylated glycans) in an umbrella topology. As will be clear to those of ordinary skill in the art, such arrays are useful for characterizing or detecting any HA polypeptides, including for example, those found in natural influenza isolates in addition to those designed and/or prepared by researchers.

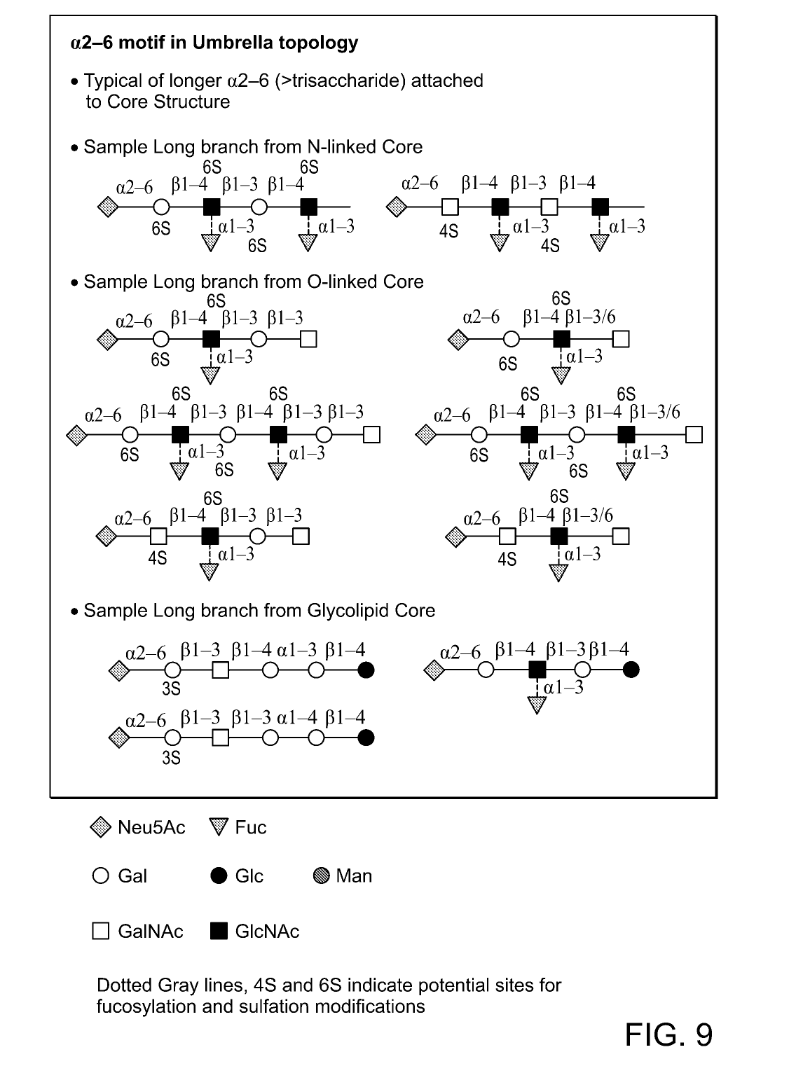

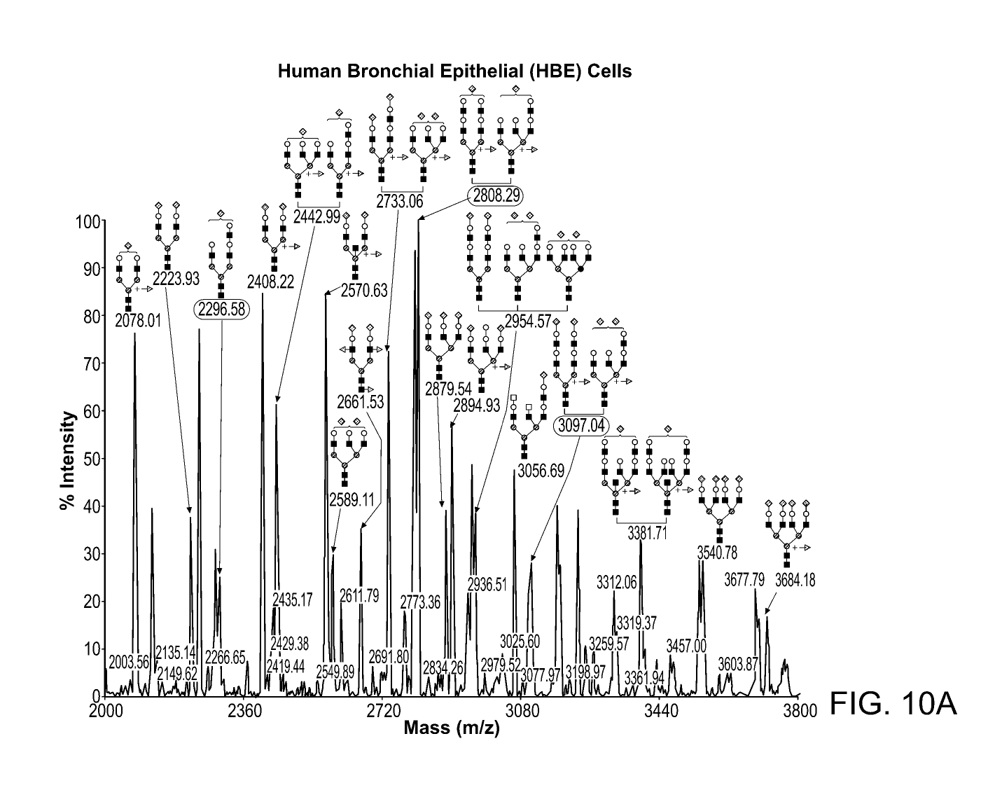

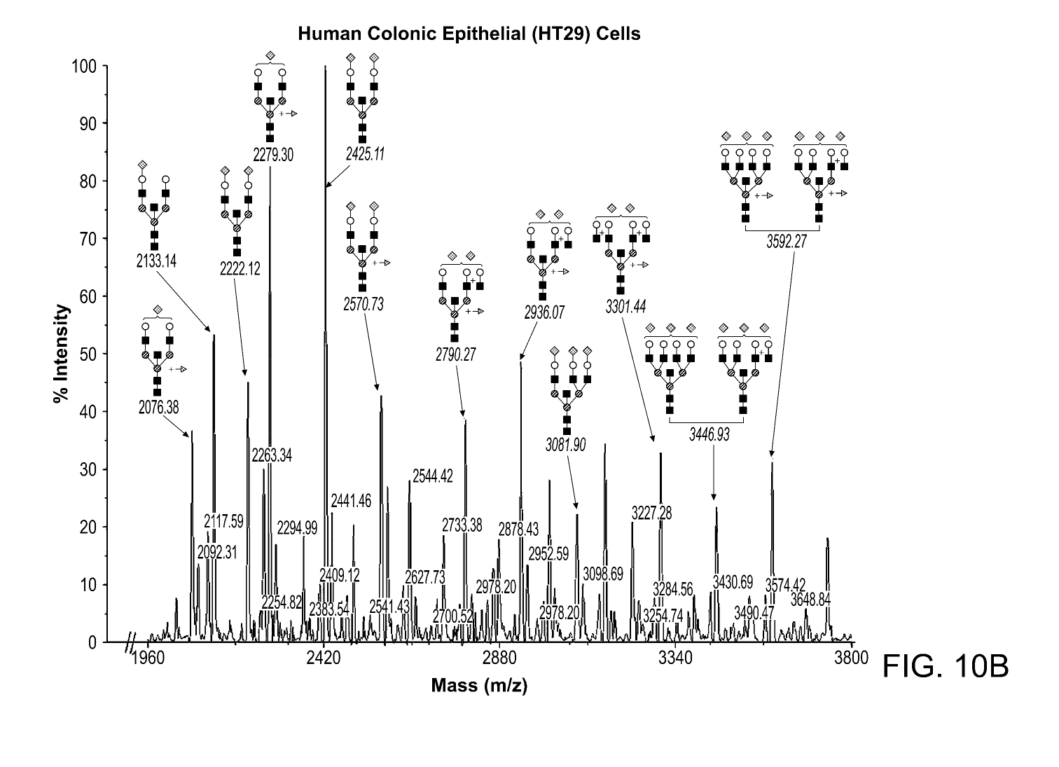

In some embodiments, such arrays include glycans representative of about 10%, 15%, 20%, 25%, 30% 35%, 40%, 45%, 50%, 55%, 60%, 65%, 70%, 75%, 80%, 85%, 90% 95%, or more of the glycans (e.g., the umbrella glycans, which will often be α2-6 sialylated glycans, particularly long α2-6 sialylated glycans) found on human HA receptors, and particularly on human upper respiratory tract HA receptors. In some embodiments, inventive arrays include some or all of the glycan structures depicted in FIG. 10 In some embodiments, arrays include at least about 10%, 15%, 20%, 25%, 30% 35%, 40%, 45%, 50%, 55%, 60%, 65%, 70%, 75%, 80%, 85%, 90% 95%, or more of these depicted glycans.

The present invention provides methods for identifying or characterizing HA proteins using glycan arrays. In some embodiments, for example, such methods comprise steps of (1) providing a sample containing HA polypeptide, (2) contacting the sample with a glycan array comprising, and (3) detecting binding of HA polypeptide to one or more glycans on the array.

Suitable sources for samples containing HA polypeptides to be contacted with glycan arrays according to the present invention include, but are not limited to, pathological samples, such as blood, serum/plasma, peripheral blood mononuclear cells/peripheral blood lymphocytes (PBMC/PBL), sputum, urine, feces, throat swabs, dermal lesion swabs, cerebrospinal fluids, cervical smears, pus samples, food matrices, and tissues from various parts of the body such as brain, spleen, and liver. Alternatively or additionally, other suitable sources for samples containing HA polypeptides include, but are not limited to, environmental samples such as soil, water, and flora. Yet other samples include laboratory samples, for example of engineered HA polypeptides designed and/or prepared by researchers. Other samples that have not been listed may also be applicable.

A wide variety of detection systems suitable for assaying HA polypeptide binding to inventive glycan arrays are known in the art. For example, HA polypeptides can be detectably labeled (directly or indirectly) prior to or after being contacted with the array; binding can then be detected by detection of localized label. In some embodiments, scanning devices can be utilized to examine particular locations on an array.

Alternatively or additionally, binding to arrayed glycans can be measured using, for example, calorimetric, fluorescence, or radioactive detection systems, or other labeling methods, or other methods that do not require labeling. In general, fluorescent detection typically involves directly probing the array with a fluorescent molecule and monitoring fluorescent signals. Alternatively or additionally, arrays can be probed with a molecule that is tagged (for example, with biotin) for indirect fluorescence detection (in this case, by testing for binding of fluorescently-labeled streptavidin). Alternatively or additionally, fluorescence quenching methods can be utilized in which the arrayed glycans are fluorescently labeled and probed with a test molecule (which may or may not be labeled with a different fluorophore). In such embodiments, binding to the array acts to squelch the fluorescence emitted from the arrayed glycan, therefore binding is detected by loss of fluorescent emission. Alternatively or additionally, arrayed glycans can be probed with a live tissue sample that has been grown in the presence of a radioactive substance, yielding a radioactively labeled probe. Binding in such embodiments can be detected by measuring radioactive emission.

Such methods are useful to determine the fact of binding and/or the extent of binding by HA polypeptides to inventive glycan arrays. In some embodiments of the invention, such methods can further be used to identify and/or characterize agents that interfere with or otherwise alter glycan-HA polypeptide interactions.

Methods described below may be of particular use in, for example, identifying whether a molecule thought to be capable of interacting with a carbohydrate can actually do so, or to identify whether a molecule unexpectedly has the capability of interacting with a carbohydrate.

The present invention also provides methods of using inventive arrays, for example, to detect a particular agent in a test sample. For instance, such methods may comprise steps of (1) contacting a glycan array with a test sample (e.g., with a sample thought to contain an HA polypeptide); and, (2) detecting the binding of any agent in the test sample to the array.

Yet further, binding to inventive arrays may be utilized, for example, to determine kinetics of interaction between binding agent and glycan. For example, inventive methods for determining interaction kinetics may include steps of (1) contacting a glycan array with the molecule being tested; and, (2) measuring kinetics of interaction between the binding agent and arrayed glycan(s).

The kinetics of interaction of a binding agent with any of the glycans in an inventive array can be measured by real time changes in, for example, colorimetric or fluorescent signals, as detailed above. Such methods may be of particular use in, for example, determining whether a particular binding agent is able to interact with a specific carbohydrate with a higher degree of binding than does a different binding agent interacting with the same carbohydrate.

It will be appreciated, of course, that glycan binding by inventive HA polypeptides can be evaluated on glycan samples or sources not present in an array format per se. For example, inventive HA polypeptides can be bound to tissue samples and/or cell lines to assess their glycan binding characteristics. Appropriate cell lines include, for example, any of a variety of mammalian cell lines, particularly those expressing HA receptors containing umbrella topology glycans (e.g., at least some of which may be α2-6 sialylated glycans, and particularly long α2-6 sialylated glycans). In some embodiments, utilized cell lines express individual glycans with umbrella topology. In some embodiments, utilized cell lines express a diversity of glycans. In some embodiments, cell lines are obtained from clinical isolates; in some they are maintained or manipulated to have a desired glycan distribution and/or prevalence. In some embodiments, tissue samples and/or cell lines express glycans characteristic of mammalian upper respiratory epithelial cells.

### Data Mining Platform

As discussed here, according to the present invention, HA polypeptides can be identified and/or characterized by mining data from glycan binding studies, structural information (e.g., HA crystal structures), and/or protein structure prediction programs.

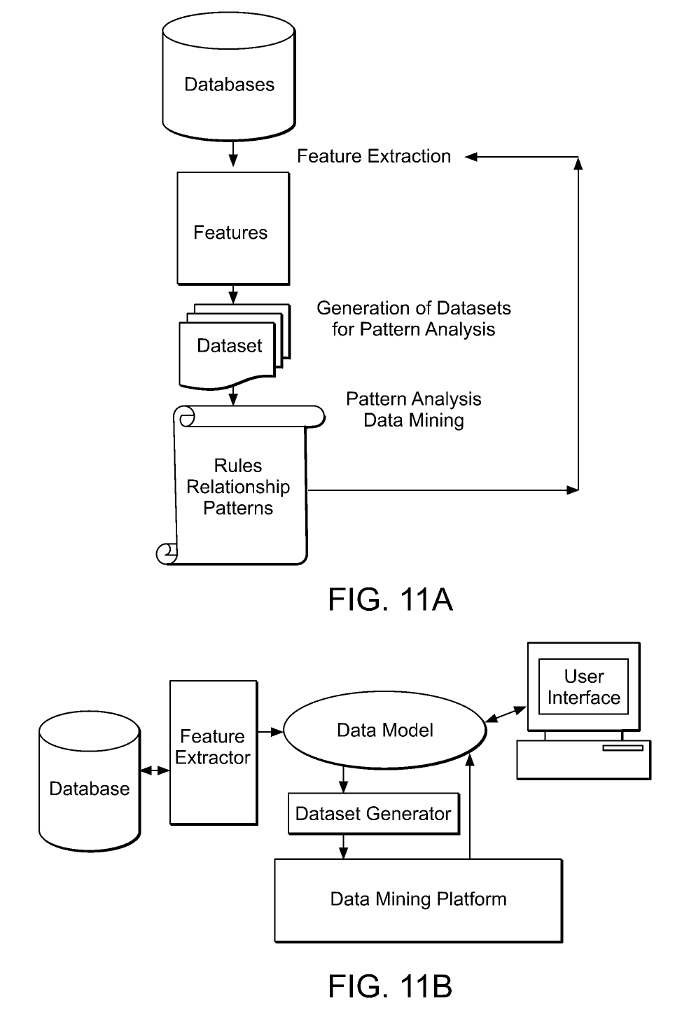

The main steps involved in the particular data mining process utilized by the present inventors (and exemplified herein) are illustrated in FIG. 11. These steps involved operations on three elements: data objects, features, and classifiers. “Data objects” were the raw data that were stored in a database. In the case of glycan array data, the chemical description of glycan structures in terms of monosaccharides and linkages and their binding signals with different GBPs screened constituted the data objects. Properties of the data objects were “features.” Rules or patterns obtained based on the features were chosen to describe a data object. “Classifiers” were the rules or patterns that were used to either cluster data objects into specific classes or determine relationships between or among features. The classifiers provided specific features that were satisfied by the glycans that bind with high affinity to a GBP. These rules were of two kinds: (1) features present on a set of high affinity glycan ligands, which can be considered to enhance binding, and (2) features that should not be present in the high affinity glycan ligands, which can be considered not favorable for binding.

The data mining platform utilized herein comprised software modules that interact with each other (FIG. 11) to perform the operations described above. The feature extractor interfaces to the CFG database to extract features, and the object-based relational database used by CFG facilitates the flexible definition of features.

### Feature Extraction and Data Preparation

Representative features extracted from the glycans on the glycan array are listed in Table 1.

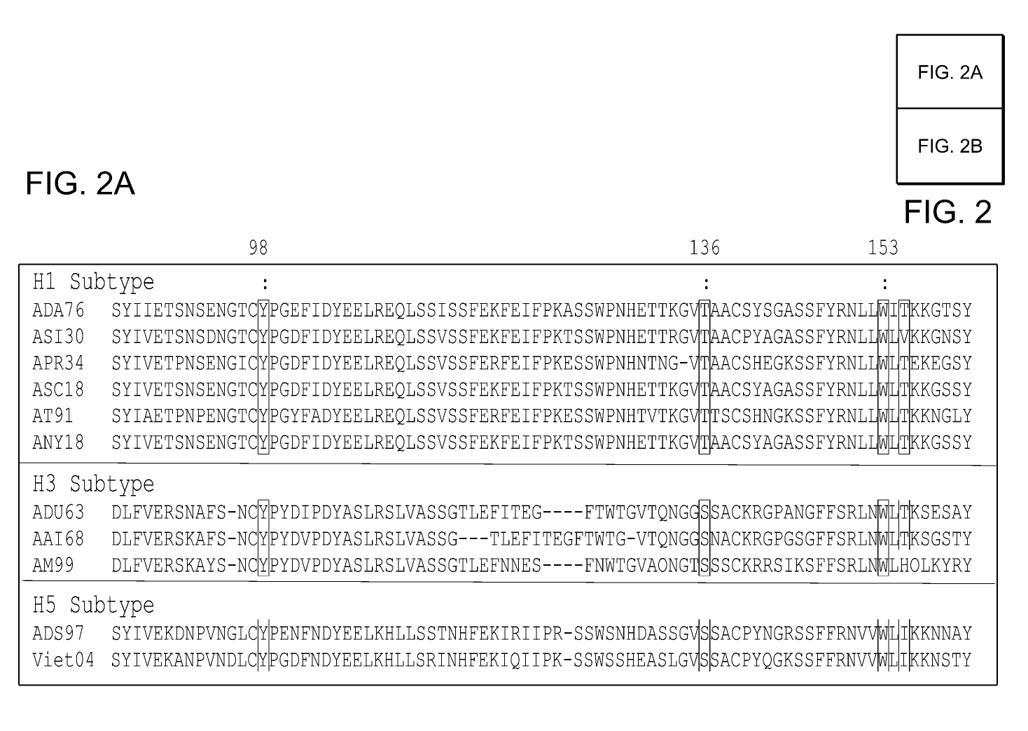

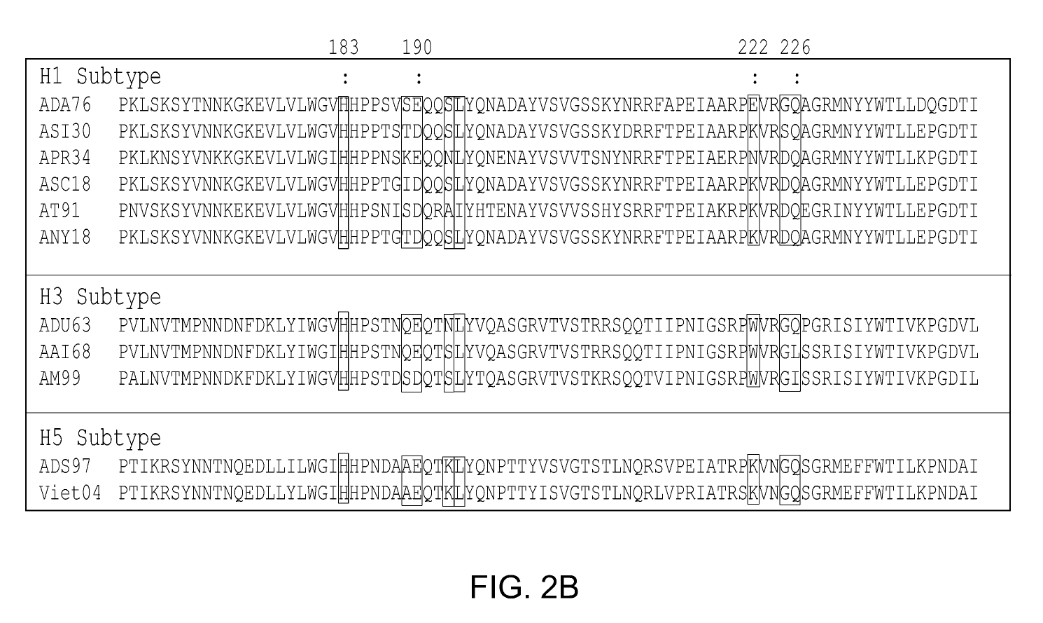

TABLE 1
Features extracted from the glycans on the glycan array.
The features described in this table were used by the rule based classification
algorithm to identify patterns that characterized binding to specific GBP.

| Features extracted | Feature Description |
|---|---|
| Monosaccharide level | |
| Composition | Number of hex, hexNAcs, dHex, sialic acids, etc [In FIG. 1, the |
| | composition is Hex = 5; HexNAc = 4]. Terminal composition is |
| | distinctly recorded [In FIG. 1, the terminal composition is |
| | Hex = 2; HexNAc = 2]. |
| Explicit Composition | Number of Glc, Gal, GlcNAc, Fuc, GalNAc, Neu5Ac, Neu5Gc, etc |
| | [In FIG. 1, the explicit composition is Man = 5; GlcNAc = 4]. Terminal |
| | explicit composition is explicitly recorded [In FIG. 1, the terminal |
| | explicit composition is Man = 2; GlcNAc = 2]. |
| Higher order features | |
| Pairs | Pair refers to a pair of monosaccharide, connected covalently by a |
| | linkage. The pairs are classified into two categories, regular [B] and |
| | terminal [T] to distinguish between the pair with one monosaccharide |
| | that terminates in the non reducing end [FIG. 2]. The frequency of |
| | the pairs were extracted as features |
| Triplets | Triplet refers to a set of three monosaccharides connected covalently |
| | by two linkages. We classify them into three categories namely |
| | regular [B], terminal [T] and surface [S] [FIG. 2]. The |
| | compositions of each category of triplets were extracted as features |
| Quadruplets | Similar to the triplet features, quadruplets features are also extracted, |
| | with four monosaccharides and their linkages [FIG. 2]. Quadruplets |
| | are classified into two varieties regular [B] and surface [S]. The |
| | frequencies of the different quadruplets were extracted as features |
| Clusters | In the case of surface triplets and quadruplets above, if the linkage |
| | information is ignored, we get a set of monosaccharide clusters, and |
| | their frequency of occurrence (composition) is tabulated. These |
| | features were chosen to analyze the importance of types of linkages |
| | between the monosaccharides. |
| Average Leaf Depth | As an indicator of the effective length of the probes, average depth of |
| | the reducing end of the tree is extracted as a glycan feature. In FIG. |
| | 2B, the leaf depths are 3, 4 and 3, and the average is 3.34 |
| Number of Leaves | As a measure of spread of the glycan tree, the number of non |
| | reducing monosaccharides is extracted as a feature. For FIG. 2B, |
| | the number of leaves is 3. For FIG. 1 it is 4. |
| GBP binding features | These features are obtained for all GBPs screened using the array |
| Mean signal per glycan | Raw signal value averaged over triplicate or quadruplicate [depending |
| | on array version] representation of the same glycan |
| Signal to Noise Ratio | Mean noise computed based on negative control [standardized |
| | method developed by CFG] to calculate signal to noise ratio [S/N] |

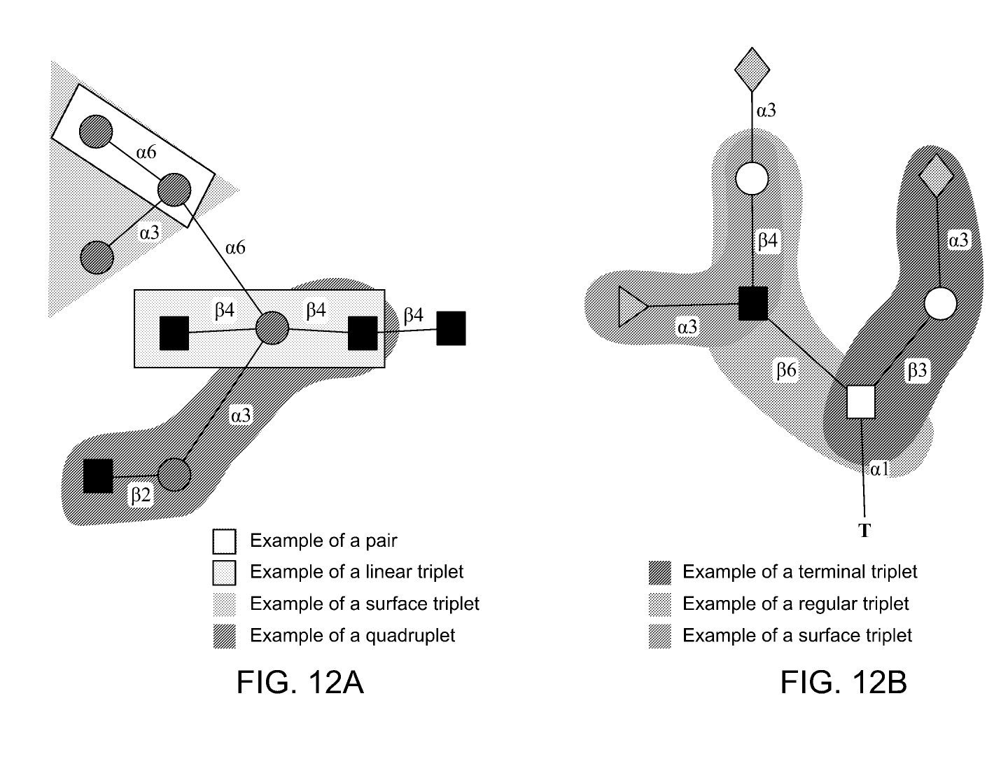

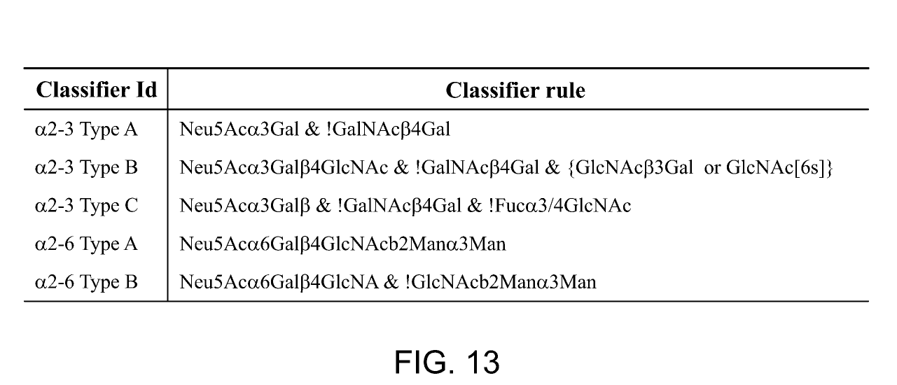

The rationale behind choosing these particular features shown was that glycan binding sites on GBPs typically accommodate di-tetra-saccharides. A tree based representation was used to capture the information on monosaccharides and linkages in the glycan structures (root of the tree at the reducing end). This representation facilitated the abstraction of various features including higher order features such as connected set of monosaccharide triplets, etc (FIG. 12). The data preparation involved generating a column-wise listing of all glycans in the glycan array along with abstracted features (Table 1) for each glycan. From this master table of glycans and their features, a subset is chosen for the rule based classification (see below) to determine specific patterns that govern the binding to a specific GBP or set of GBPs.

### Classifiers

Different types of classifiers have been developed and used in many applications. They fall primarily into three main categories: Mathematical Methods, Distance Methods and Logic Methods. These different methods and their advantages and disadvantages are discussed in detail in Weiss & Indrukhya (Predictive data mining—A practical guide. Morgan Kaufmann, Sann Francisco, 1998). For this specific application we chose a method called Rule Induction, which falls under Logic Methods. The Rule Induction classifier generates patterns in form of IF-THEN rules.

One of the main advantages of the Logic Methods, and specifically classifiers such as the Rule Induction method that generate IF-THEN rules, is that the results of the classifiers can be explained more easily when compared to the other statistical or mathematical methods. This allows one to explore the structural and biological significance of the rule or pattern discovered. An example rule generated using the features described earlier (Table 1) is: IF A Glycan contains “Galb4GlcNAcb3Gal[B]” and DOES NOT contain “Fuca3GlcNAc[B]”, THEN the Glycan will bind with higher affinity to Galectin 3. The specific Rule Induction algorithm that was used in this case is the one developed by Weiss & Indurkya (Predictive data mining—A practical guide. Morgan Kaufmann, Sann Francisco, 1998.

### Binding Levels

A threshold that distinguished low affinity and high affinity binding was defined for each of the glycan array screening data sets.

### Nucleic Acids

In certain embodiments, the present invention provides nucleic acids which encode an HA polypeptide or a characteristic or biologically active portion of an HA polypeptide. In other embodiments, the invention provides nucleic acids which are complementary to nucleic acids which encode an HA polypeptide or a characteristic or biologically active portion of an HA polypeptide.

In other embodiments, the invention provides nucleic acid molecules which hybridize to nucleic acids encoding an HA polypeptide or a characteristic or biologically active portion of an HA polypeptide. Such nucleic acids can be used, for example, as primers or as probes. To give but a few examples, such nucleic acids can be used as primersin polymerase chain reaction (PCR), as probes for hybridization (including in situ hybridization), and/or as primers for reverse transcription-PCR (RT-PCR).

In certain embodiments, nucleic acids can be DNA or RNA, and can be single stranded or double-stranded. In some embodiments, inventive nucleic acids may include one or more non-natural nucleotides; in other embodiments, inventive nucleic acids include only natural nucleotides.

### Antibodies

The present invention provides antibodies to inventive HA polypeptides. These may be monoclonal or polyclonal and may be prepared by any of a variety of techniques known to those of ordinary skill in the art (e.g., see Harlow and Lane, Antibodies: A Laboratory Manual, Cold Spring Harbor Laboratory, 1988). For example, antibodies can be produced by cell culture techniques, including the generation of monoclonal antibodies, or via transfection of antibody genes into suitable bacterial or mammalian cell hosts, in order to allow for the production of recombinant antibodies.

### Pharmaceutical Compositions

In some embodiments, the present invention provides for pharmaceutical compositions including HA polypeptide(s), nucleic acids encoding such polypeptides, characteristic or biologically active fragments of such polypeptideds or nucleic acids, antibodies that bind to such polypeptides or fragments, small molecules that interact with such polypeptides or with glycans that bind to them, etc.

The invention encompasses treatment of influenza infections by administration of such inventive pharmaceutical compositions. In some embodiments, treatment is accomplished by administration of a vaccine. To date, although significant accomplishments have been made in the development of influenza vaccines, there is room for further improvement. The present invention provides vaccines comprising inventive HA polypeptides, and particularly comprising HA polypeptides that bind to umbrella glycans (e.g., α2-6 linked umbrella glycans such as, for example, long α2-6 sialylated glycans).

To give but one example, attempts to generate vaccines specific for the H5N1 strain in humans have generally not been successful due, at least in part, to low immunogenicity of H5 HAs. In one study, a vaccine directed at the H5N1 strain was shown to yield antibody titers of 1:40, which is not a titer high enough to guarantee protection from infection. Furthermore, the dosage required to generate even a modest 1:40 antibody titer (two doses of 90 μg of purified killed virus or antigen) was 12-times that normally used in the case of the common seasonal influenza virus vaccine (Treanor et al., N Eng J Med, 354:1343, 2006). Other studies have similarly shown that current H5 vaccines are not highly immunogenic (Bresson et al., Lancet, 367:1657, 2006). In some embodiments, inventive vaccines are formulated utilizing one or more strategies (see, for example, Enserink, Science, 309:996, 2005) intended to allow use of lower dose of H5 HA protein, and/or to achieve higher immunogenicity. For example, in some embodiments, multivalency is improved (e.g., via use of dendrimers); in some embodiments, one or more adjuvants is utilized, etc.

In some embodiments, the present invention provides for vaccines and the administration of these vaccines to a human subject. In certain embodiments, vaccines are compositions comprising one or more of the following: (1) inactivated virus, (2) live attenuated influenza virus, for example, replication-defective virus, (3) inventive HA polypeptide or characteristic or biologically active portion thereof, (4) nucleic acid encoding HA polypeptide or characteristic or biologically active portion thereof, (5) DNA vector that encodes HA polypeptide or characteristic or biologically active portion thereof, and/or (6) expression system, for example, cells expressing one or more influenza proteins to be used as antigens.

Thus, in some embodiments, the present invention provides inactivated flu vaccines. In certain embodiments, inactivated flu vaccines comprise one of three types of antigen preparation: inactivated whole virus, sub-virions where purified virus particles are disrupted with detergents or other reagents to solubilize the lipid envelope (“split” vaccine) or purified HA polypeptide (“subunit” vaccine). In certain embodiments, virus can be inactivated by treatment with formaldehyde, beta-propiolactone, ether, ether with detergent (such as Tween-80), cetyl trimethyl ammonium bromide (CTAB) and Triton N101, sodium deoxycholate and tri(n-butyl) phosphate. Inactivation can occur after or prior to clarification of allantoic fluid (from virus produced in eggs); the virions are isolated and purified by centrifugation (Nicholson et al., eds., Textbook of Influenza, Blackwell Science, Malden, Mass., 1998). To assess the potency of the vaccine, the single radial immunodiffusion (SRD) test can be used (Schild et al., Bull. World Health Organ., 52:43-50 & 223-31, 1975; Mostow et al., J. Clin. Microbiol., 2:531, 1975).

The present invention also provides live, attenuated flu vaccines, and methods for attenuation are well known in the art. In certain embodiments, attenuation is achieved through the use of reverse genetics, such as site-directed mutagenesis.

In some embodiments, influenza virus for use in vaccines is grown in eggs, for example, in embryonated hen eggs, in which case the harvested material is allantoic fluid. Alternatively or additionally, influenza virus may be derived from any method using tissue culture to grow the virus. Suitable cell substrates for growing the virus include, for example, dog kidney cells such as MDCK or cells from a clone of MDCK, MDCK-like cells, monkey kidney cells such as AGMK cells including Vero cells, cultured epithelial cells as continuous cell lines, 293T cells, BK-21 cells, CV-1 cells, or any other mammalian cell type suitable for the production of influenza virus (including upper airway epithelial cells) for vaccine purposes, readily available from commercial sources (e.g., ATCC, Rockville, Md.). Suitable cell substrates also include human cells such as MRC-5 cells. Suitable cell substrates are not limited to cell lines; for example primary cells such as chicken embryo fibroblasts are also included.

In some embodiments, inventive vaccines further comprise one or more adjuvants. For example, aluminum salts (Baylor et al., Vaccine, 20:S18, 2002) and monophosphoryl lipid A (MPL; Ribi et al., (1986, Immunology and Immunopharmacology of bacterial endotoxins, Plenum Publ. Corp., NY, p 407, 1986) can be used as adjuvants in human vaccines. Alternatively or additionally, new compounds are currently being tested as adjuvants in human vaccines, such as MF59 (Chiron Corp., http://www.chiron.com/investors/pressreleases/2005/051028.html), CPG 7909 (Cooper et al., Vaccine, 22:3136, 2004), and saponins, such as QS21 (Ghochikyan et al., Vaccine, 24:2275, 2006).

Additionally, some adjuvants are known in the art to enhance the immunogenicity of influenza vaccines, such as poly[di(carboxylatophenoxy)phosphazene] (PCCP; Payne et al., Vaccine, 16:92, 1998), interferon-γ (Cao et al., Vaccine, 10:238, 1992), block copolymer P1205 (CRL1005; Katz et al., Vaccine, 18:2177, 2000), interleukin-2 (IL-2; Mbwuike et al., Vaccine, 8:347, 1990), and polymethyl methacrylate (PMMA; Kreuter et al., J. Pharm. Sci., 70:367, 1981).

In addition to vaccines, the present invention provides other therapeutic compositions useful in the treatment of viral infections. For example, in some embodiments, treatment is accomplished by administration of an agent that interferes with expression or activity of an inventive HA polypeptide. For example, treatment can be accomplished with a composition comprising antibodies (such as antibodies that recognize virus particles containing a particular HA polypeptide (e.g., an HA polypeptide that binds to umbrella glycans), nucleic acids (such as nucleic acid sequences complementary to HA sequences, which can be used for RNAi), glycans that compete for binding to HA receptors, small molecules or glycomometics that compete the glycan-HA polypeptide interaction, or any combination thereof. In some embodiments, collections of different agents, having diverse structures are utilized. In some embodiments, therapeutic compositions comprise one or more multivalent agents. In some embodiments, treatment comprises urgent administration shortly after exposure or suspicion of exposure.

In general, a pharmaceutical composition will include a therapeutic agent in addition to one or more inactive agents such as a sterile, biocompatible carrier including, but not limited to, sterile water, saline, buffered saline, or dextrose solution. Alternatively or additionally, the composition can contain any of a variety of additives, such as stabilizers, buffers, excipients, or preservatives. In certain embodiments, a pharmaceutical composition will include a therapeutic agent that is encapsulated, trapped, or bound within a lipid vesicle, a bioavailable and/or biocompatible and/or biodegradable matrix, or other microparticle.

The pharmaceutical compositions of the present invention may be administered either alone or in combination with one or more other therapeutic agents including, but not limited to, vaccines and/or antibodies. By “in combination with,” it is not intended to imply that the agents must be administered at the same time or formulated for delivery together, although these methods of delivery are within the scope of the invention. In general, each agent will be administered at a dose and on a time schedule determined for that agent. Additionally, the invention encompasses the delivery of the inventive pharmaceutical compositions in combination with agents that may improve their bioavailability, reduce or modify their metabolism, inhibit their excretion, or modify their distribution within the body. Although the pharmaceutical compositions of the present invention can be used for treatment of any subject (e.g., any animal) in need thereof, they are most preferably used in the treatment of humans.

The pharmaceutical compositions of the present invention can be administered by a variety of routes, including oral, intravenous, intramuscular, intra-arterial, subcutaneous, intraventricular, transdermal, interdermal, rectal, intravaginal, intraperitoneal, topical (as by powders, ointments, creams, or drops), mucosal, bucal, or as an oral or nasal spray or aerosol. In general the most appropriate route of administration will depend upon a variety of factors including the nature of the agent (e.g., its stability in the environment of the gastrointestinal tract), the condition of the patient (e.g., whether the patient is able to tolerate oral administration), etc. At present the oral or nasal spray or aerosol route is most commonly used to deliver therapeutic agents directly to the lungs and respiratory system. However, the invention encompasses the delivery of the inventive pharmaceutical composition by any appropriate route taking into consideration likely advances in the sciences of drug delivery.

Suitable devices for use in delivering intradermal pharmaceutical compositions described herein include short needle devices such as those described in U.S. Pat. No. 4,886,499, U.S. Pat. No. 5,190,521, U.S. Pat. No. 5,328,483, U.S. Pat. No. 5,527,288, U.S. Pat. No. 4,270,537, U.S. Pat. No. 5,015,235, U.S. Pat. No. 5,141,496, U.S. Pat. No. 5,417,662. Intradermal compositions may also be administered by devices which limit the effective penetration length of a needle into the skin, such as those described in WO99/34850, incorporated herein by reference, and functional equivalents thereof. Also suitable are jet injection devices which deliver liquid vaccines to the dermis via a liquid jet injector or via a needle which pierces the stratum corneum and produces a jet which reaches the dermis. Jet injection devices are described for example in U.S. Pat. No. 5,480,381, U.S. Pat. No. 5,599,302, U.S. Pat. No. 5,334,144, U.S. Pat. No. 5,993,412, U.S. Pat. No. 5,649,912, U.S. Pat. No. 5,569,189, U.S. Pat. No. 5,704,911, U.S. Pat. No. 5,383,851, U.S. Pat. No. 5,893,397, U.S. Pat. No. 5,466,220, U.S. Pat. No. 5,339,163, U.S. Pat. No. 5,312,335, U.S. Pat. No. 5,503,627, U.S. Pat. No. 5,064,413, U.S. Pat. No. 5,520,639, U.S. Pat. No. 4,596,556, U.S. Pat. No. 4,790,824, U.S. Pat. No. 4,941,880, U.S. Pat. No. 4,940,460, WO 97/37705 and WO 97/13537. Also suitable are ballistic powder/particle delivery devices which use compressed gas to accelerate vaccine in powder form through the outer layers of the skin to the dermis. Additionally, conventional syringes may be used in the classical mantoux method of intradermal administration.

General considerations in the formulation and manufacture of pharmaceutical agents may be found, for example, in Remington's Pharmaceutical Sciences, 19^{th} ed., Mack Publishing Co., Easton, Pa., 1995.

### Diagnostics/Kits

The present invention provides kits for detecting HA polypeptides, and particular for detecting HA polypeptides with particular glycan binding characteristics (e.g., binding to umbrella glycans, to α2-6 sialylated glycans, to long α2-6 sialylated glycans, etc.) in pathological samples, including, but not limited to, blood, serum/plasma, peripheral blood mononuclear cells/peripheral blood lymphocytes (PBMC/PBL), sputum, urine, feces, throat swabs, dermal lesion swabs, cerebrospinal fluids, cervical smears, pus samples, food matrices, and tissues from various parts of the body such as brain, spleen, and liver. The present invention also provides kits for detecting HA polypeptides of interest in environmental samples, including, but not limited to, soil, water, and flora. Other samples that have not been listed may also be applicable.

In certain embodiments, inventive kits may include one or more agents that specifically detect HA polypeptides with particular glycan binding characteristics. Such agents may include, for example, antibodies that specifically recognize certain HA polypeptides (e.g., HA polypeptides that bind to umbrella glycans and/or to α2-6 sialylated glycans and/or to long α2-6 sialylated glycans), which can be used to specifically detect such HA polypeptides by ELISA, immunofluorescence, and/or immunoblotting. These antibodies can also be used in virus neutralization tests, in which a sample is treated with antibody specific to HA polypeptides of interest, and tested for its ability to infect cultured cells relative to untreated sample. If the virus in that sample contains such HA polypeptides, the antibody will neutralize the virus and prevent it from infecting the cultured cells. Alternatively or additionally, such antibodies can also be used in HA-inhibition tests, in which the HA protein is isolated from a given sample, treated with antibody specific to a particular HA polypeptide or set of HA polypeptides, and tested for its ability to agglutinate erythrocytes relative to untreated sample. If the virus in the sample contains such an HA polypeptide, the antibody will neutralize the activity of the HA polypeptide and prevent it from agglutinating erythrocytes (Harlow & Lane, Antibodies: A Laboratory Manual, CSHL Press, 1988; http://www.who.int/csr/resources/publications/influenza/WHO_CDS_CSR_NCS_{—}2002_{—}5/en/index.html; http://www.who.int/csr/disease/avian_influenza/guidelines/labtests/en/index.html). In other embodiments, such agents may include nucleic acids that specifically bind to nucleotides that encode particular HA polypeptides and that can be used to specifically detect such HA polypeptides by RT-PCR or in situ hybridization (http://www.who.int/csr/resources/publications/influenza/WHO_CDS_CSR_NCS_{—}2002_{—}5/en/index.html; http://www.who.int/csr/disease/avian_influenza/guidelines/labtests/en/index.html). In certain embodiments, nucleic acids which have been isolated from a sample are amplified prior to detection. In certain embodiments, diagnostic reagents can be detectably labeled.

The present invention also provides kits containing reagents according to the invention for the generation of influenza viruses and vaccines. Contents of the kits include, but are not limited to, expression plasmids containing the HA nucleotides (or characteristic or biologically active portions) encoding HA polypeptides of interest (or characteristic or biologically active portions). Alternatively or additionally, kits may contain expression plasmids that express HA polypeptides of interest (or characteristic or biologically active portions). Expression plasmids containing no virus genes may also be included so that users are capable of incorporating HA nucleotides from any influenza virus of interest. Mammalian cell lines may also be included with the kits, including but not limited to, Vero and MDCK cell lines. In certain embodiments, diagnostic reagents can be detectably labeled.

In certain embodiments, kits for use in accordance with the present invention may include, a reference sample, instructions for processing samples, performing the test, instructions for interpreting the results, buffers and/or other reagents necessary for performing the test. In certain embodiments the kit can comprise a panel of antibodies.

In some embodiments of the present invention, glycan arrays, as discussed above, may be utilized as diagnostics and/or kits.

In certain embodiments, inventive glycan arrays and/or kits are used to perform dose response studies to assess binding of HA polypeptides to umbrella glycans at multiple doses (e.g., as described herein). Such studies give particularly valuable insight into the binding characteristics of tested HA polypeptides, and are particularly useful to assess specific binding. Dose response binding studies of this type find many useful applications. To give but one example, they can be helpful in tracking the evolution of binding characteristics in a related series of HA polypeptide variants, whether the series is generated through natural evolution, intentional engineering, or a combination of the two.

In certain embodiments, inventive glycan arrays and/or kits are used to induce, identify, and/or select HA polypeptides, and/or HA polypeptide variants having desired binding characteristics. For instance, in some embodiments, inventive glycan arrays and/or kits are used to exert evolutionary (e.g., screening and/or selection) pressure on a population of HA polypeptides.

### EXEMPLIFICATION

### Example 1

### Framework for Binding Specificity of H1, H3 and H5 HAs to α2-3 and α2-6 Sialylated Glycans

Crystal structures of HAs from H1 (PDB IDS: 1RD8, 1RU7, 1RUY, 1RV0, 1RVT, 1RVX, 1RVZ), H3 (PDB IDs: 1MQL, 1MQM, 1MQN) and H5 (1JSN, 1JSO, 2FK0) and their complexes with α2-3 and/or α2-6 sialylated oligosaccharides have provided molecular insights into residues involved in specific HA-glycan interactions. More recently, the glycan receptor specificity of avian and human H1 and H3 subtypes has been elaborated by screening the wild type and mutants on glycan arrays comprising of a variety of α2-3 and α2-6 sialylated glycans.

The Asp190Glu mutation in the HA of the 1918 human pandemic virus reversed its specificity from α2-6 to α2-3 sialylated glycans (Stevens et al., J. Mol. Biol., 355:1143, 2006; Glaser et al., J. Virol., 79:11533, 2005). On the other hand, the double mutation Glu190Asp and Gly225Asp on an avian H1 (A/Duck/Alberta/35/1976) reversed its specificity from α2-3 to α2-6 sialylated glycans. In the case of the H3 subtype, the amino acid changes from Gln226 to Leu and Gly228 to Ser between the 1963 avian H3N8 strain and the 1967-68 pandemic human H3N2 strain correlate with the change in their preference from α2-3 to α2-6 sialylated glycans (Rogers et al., Nature, 304:76, 1983). The relationship between the HA glycan binding specificity and transmission efficiency was demonstrated in a ferret model using the highly pathogenic and virulent 1918 H1N1 viruses (Tumpey, T. M. et al. Science 315: 655, 2007).

Switching the receptor binding specificity from the parental human α2-6 sialylated glycan (SC18) receptor preference to an avian α2-3 sialylated receptor preference (AV18) resulted in a virus that was unable to transmit. On the other hand, one of the mixed α2-3/α2-6 sialylated glycan specificity virus (A/New York/1/8 (NY18)) showed no transmission, surprisingly A/Texas/36/91 (Tx91) virus, also mixed α2-3/α2-6 sialylated glycan specificity, was able to efficiently transmit. Furthermore, as stated above, various strains of the highly pathogenic H5N1 viruses also show mixed α2-3/α2-6 sialylated glycan specificity (Yamada, S. et al. Nature 444:378, 2006), and have yet been able to transmit from human-to-human. The confounding results with respect to HA's sialylated glycan specificity and transmission posed the following questions. First, is there diversity in the sialylated glycans found in the upper airways in humans, and could that account for the specificity and tissue tropism of the virus? Second, are there nuances of glycan conformation that might play a role in how both α2-3 and/or α2-6 sialylated glycans bind to HA glycan binding pocket? Taken together, what are the glycan binding requirements of the Influenza A virus HA for human adaptation?

### Structural Constraints Imposed by Glycan Topology and Substitutions on H1, H3 and H5 HA Binding to α2-3 Sialylated Glycans

Analysis of all the HA-glycan co-crystal structures indicates that the orientation of the Neu5Ac sugar (SA) is fixed relative to the HA glycan binding site. A highly conserved set of amino acids Phe95, Ser/Thr136, Trp153, His183, Leu/Ile194 across different HA subtypes are involved in anchoring the SA. Therefore, the specificity of HA to α2-3 or α2-6 is governed by interactions of the HA glycan binding site with the glycosidic oxygen atom and sugars beyond SA.

The conformation of the Neu5Acα2-3Gal linkage is such that the positioning of Gal and sugars beyond Gal in α2-3 fall in a cone-like region governed by the glycosidic torsion angles at this linkage (FIG. 6). The typical region of minimum energy conformations is given by +values of around −60 or 60 or 180 where ψ samples −60 to 60 (FIG. 14). In these minimum energy regions, the sugars beyond Gal in α2-3 are projected out of the HA glycan binding site. This is also evident from the co-crystal structures of HA with the α2-3 motif (Neu5Acα2-3Galβ1-3/4GlcNAc-) where the φ value is typically around 180 (referred to as trans conformation). The trans conformation causes the α2-3 motif to project out of the pocket. This implies that structural variations (sulfation and fucosylation) branching at the Gal and/or GlcNAc (or GalNAc) sugars centered on the three sugar (or trisaccharide) α2-3 motif will have the most influence on the HA binding (FIG. 7). This structural implication is consistent with the three distinct classifiers for HA binding to α2-3 sialylated glycans obtained from the data mining analysis (Table 3). The common feature in all these three classes is that the Neu5Acα2-3Gal should not be present along with a GalNAcα/β1-4Gal. Analysis of the crystal structures showed that the GalNAc linked to Gal of Neu5Acα2-3Gal made unfavorable steric contacts with the protein, consistent with the classifiers.

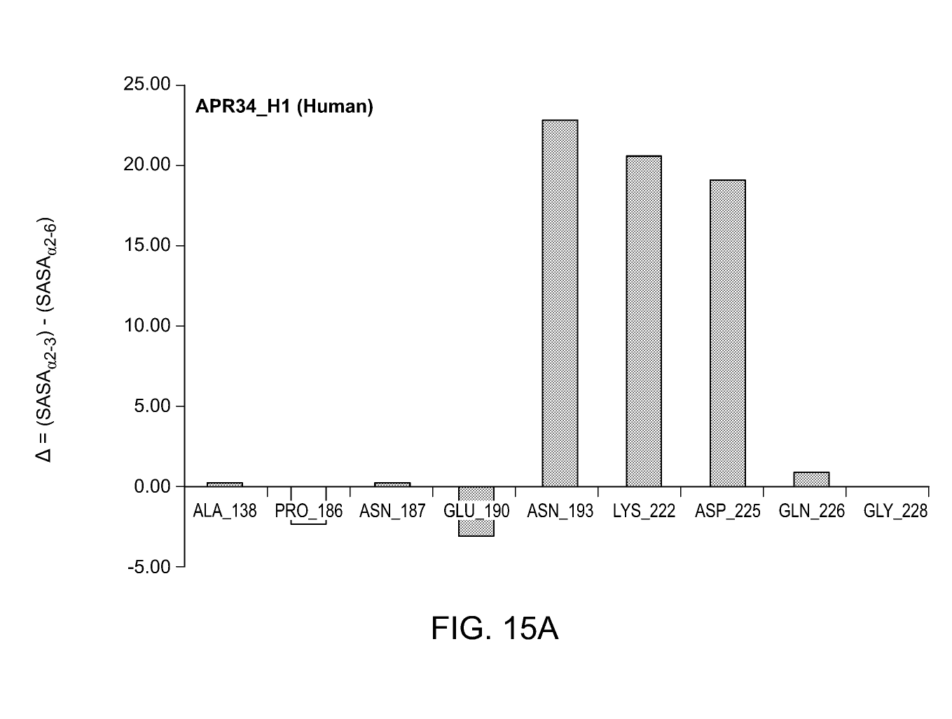

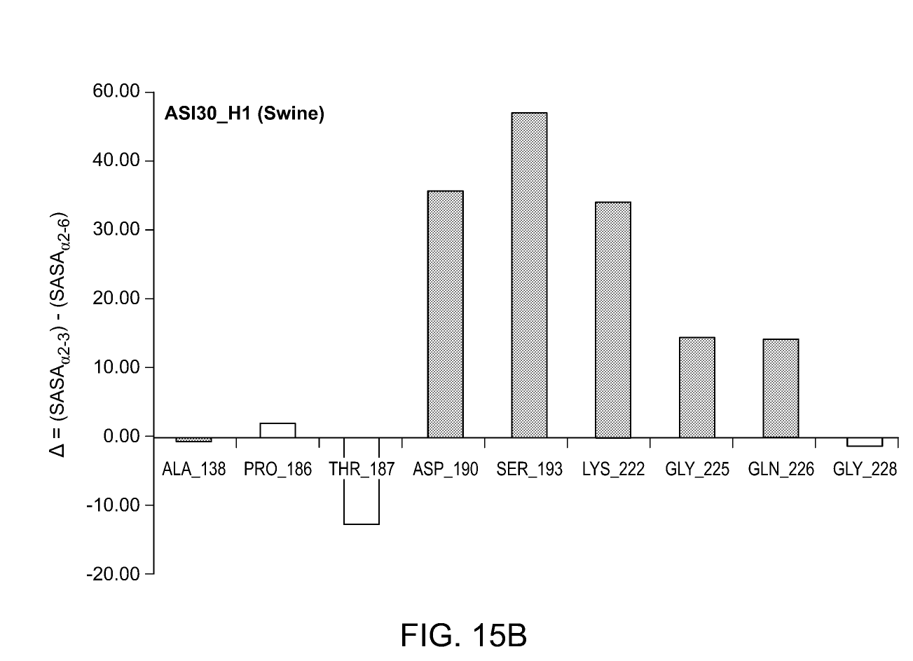

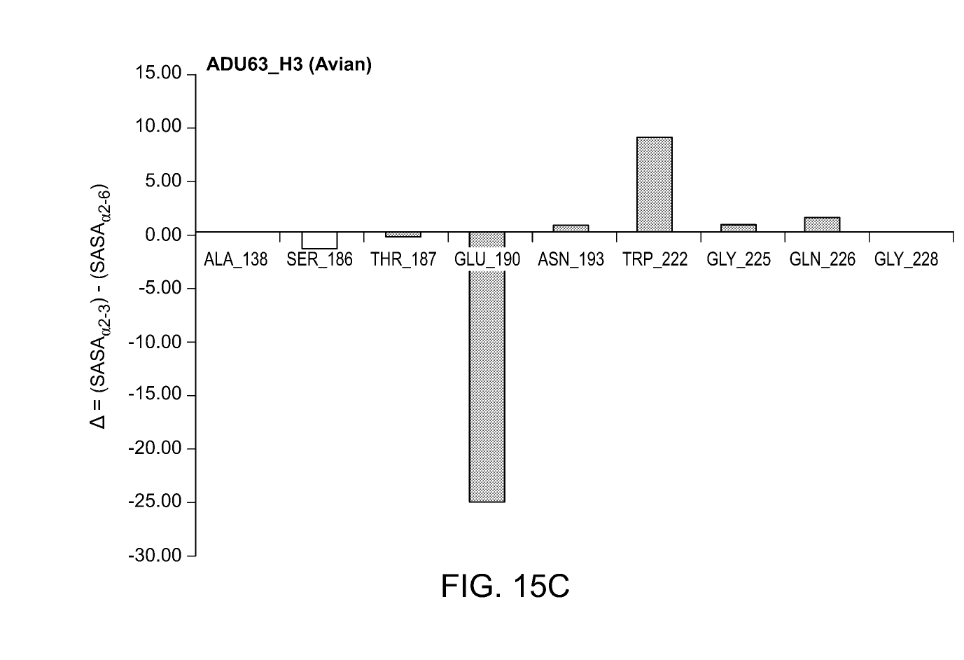

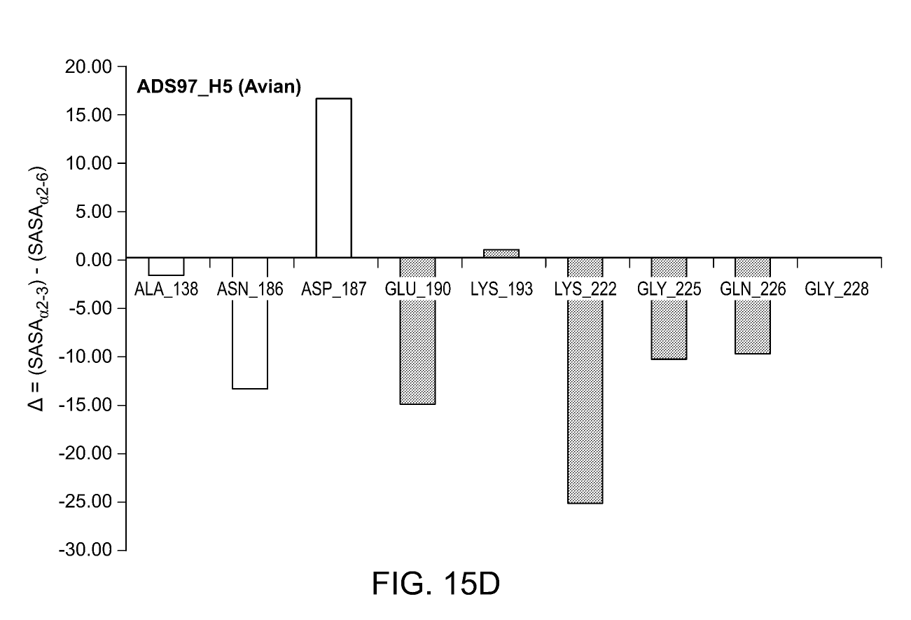

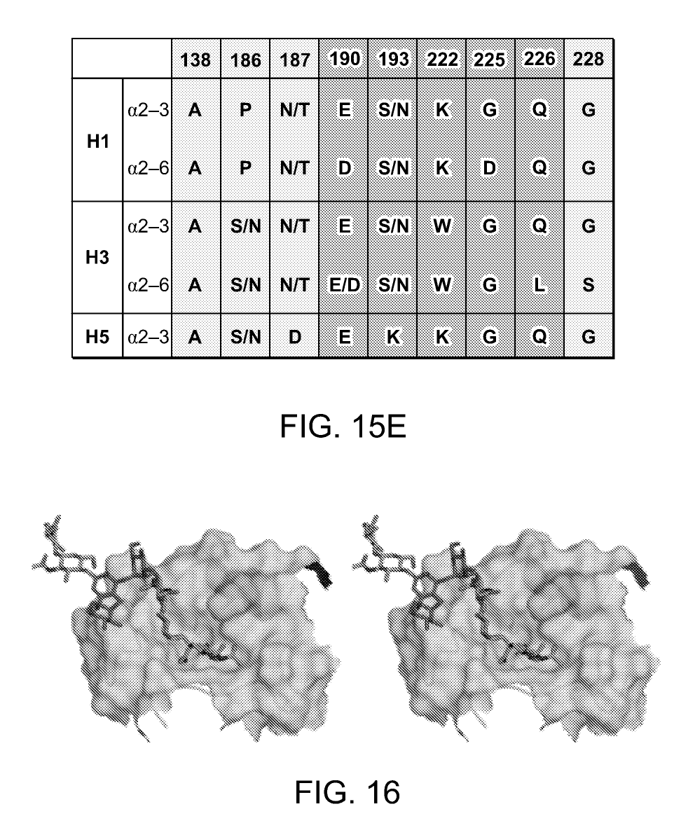

In addition to the conserved anchor points for sialic acid binding, two critical residues, Gln226 and Glu190, are involved in binding to the Neu5Acα2-3Gal motif. Gln226, located at the base of the binding site, interacts with the glycosidic oxygen atom of the Neu5Acα2-3Gal linkage (FIG. 15, Panels C,D). Glu190, located on the opposite side of Gln226 interacts with Neu5Ac and Gal monosaccharides (FIG. 15, Panels C,D). Further, residues Ala138 (proximal to Gln226) and Gly228 (proximal to Glu190), which are highly conserved in avian HAs could be involved in facilitating the right conformation of Gln226 and Glu190 for optimal interactions with α2-3 sialylated glycans (FIG. 15). APR34, a human H1 subtype, contains all the four amino acids Ala138, Glu190, Gln226 and Gly228 and binds to α2-3 sialylated glycans as observed in its crystal structure (FIG. 14, Panel B).

Superimposition of the glycan binding site in the crystal structures of AAI68_H3_{—}23, ADU67_H3_{—}23 and APR34_H1_{—}23 gaves additional insights into the positioning of the Glu190 side chain and its effect on HA binding to α2-3 sialylated glycans. The side chain of Glu190 in H1 HA is further (around 1 Å) into the binding site in comparison with that of Glu190 in H3 HA. This could be due to the amino acid differences Pro186 in H1 HA as against Ser186 in H3 HA which are proximal to the Glu190 residue. This change in side chain conformation of Glu190 could correlate with the binding of avian H1 (and not avian H3) with moderate affinity to some of the α2-6 sialylated glycans as shown by the data mining analysis of the glycan microarray data (Table 3). Further, substitution of Gly228 to Ser—a hallmark change between avian and human H3 subtypes—alters the conformation of Glu190 and interferes with the interaction of human H3 HA to Neu5Acα2-3Gal in the trans conformation. This is further elaborated by the distinct conformation (that is not trans) of Neu5Acα2-3Gal motif observed in the human AAI68_H3_{—}23 co-crystal structure. The Neu5Acα2-3 Gal motif in this conformation provides less optimal contacts with human H3 HA binding site compared to those provided by this motif in the trans conformation with the avian H3 HA (FIG. 14). As a consequence of this loss of contacts, the Gly228Ser mutation in human H3 HA makes its glycan binding site less favorable for interaction with α2-3 sialylated glycans. This structural observation is consistent with the results from the data mining analysis (Table 3) which shows that the human H3 HA has only a moderate affinity for some of the α2-3 sialylated glycans.

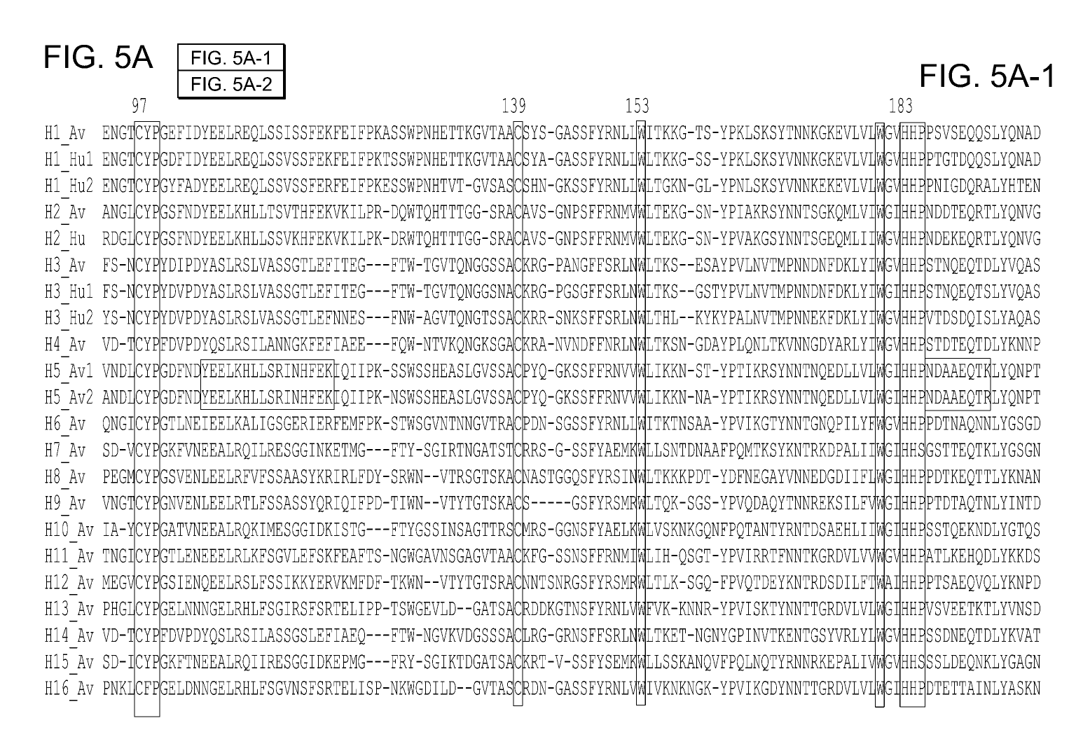

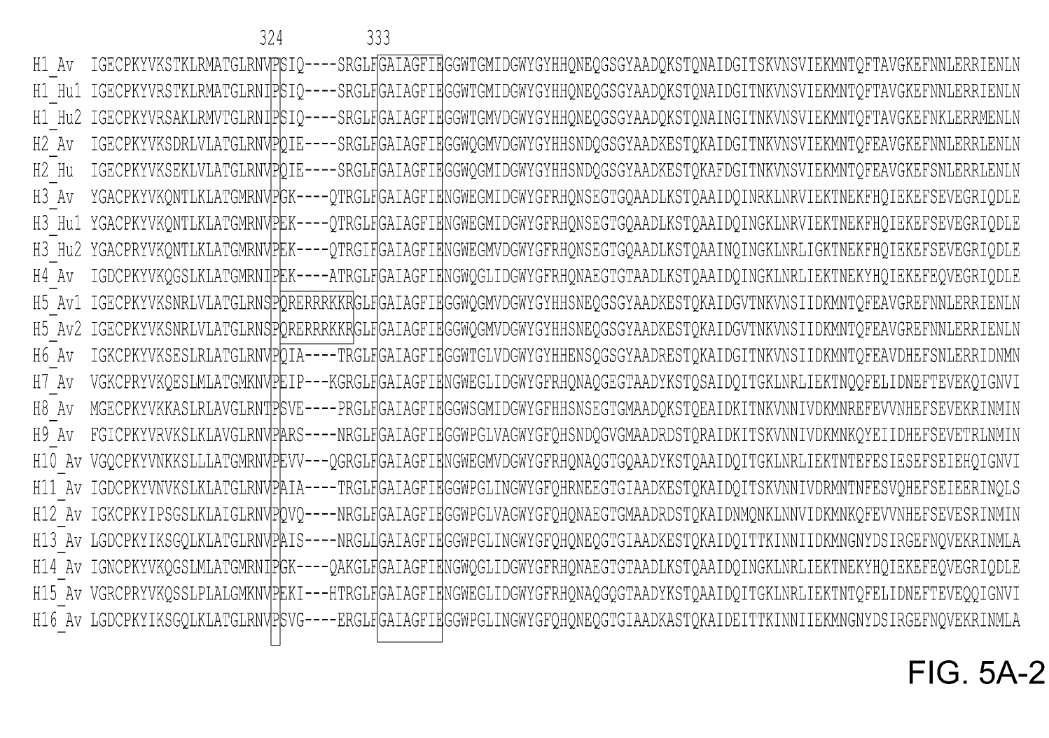

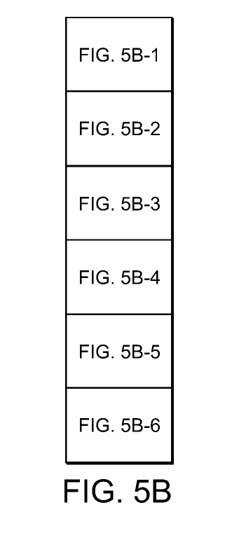

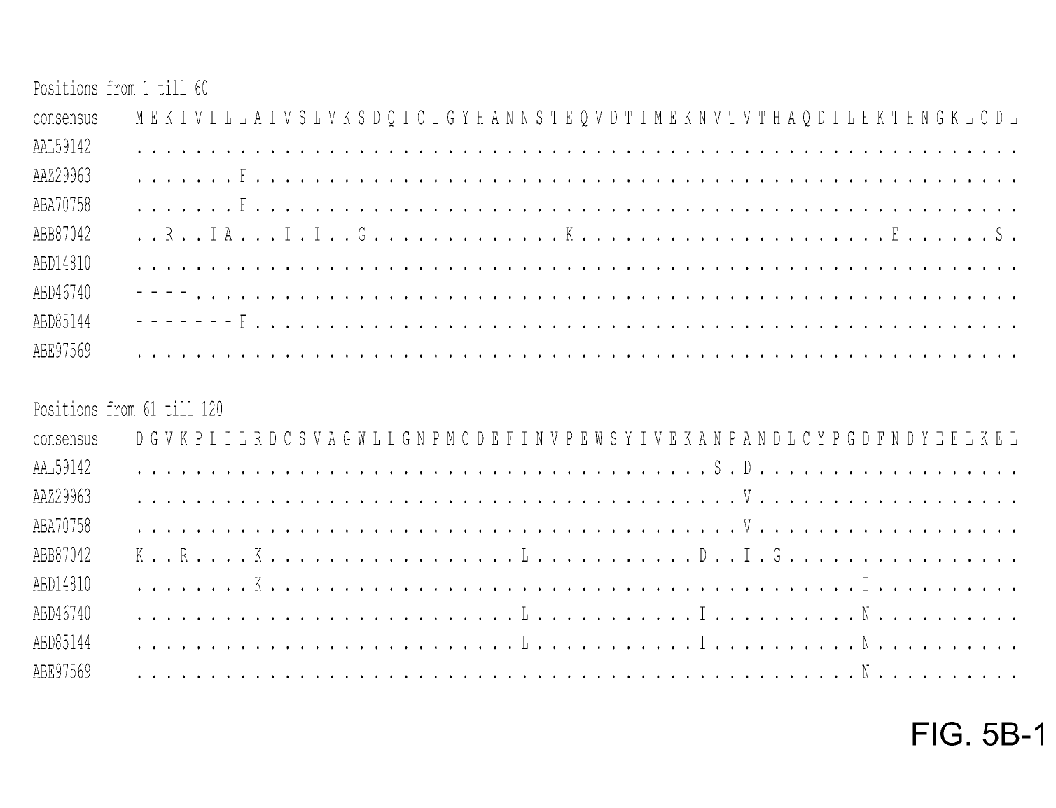

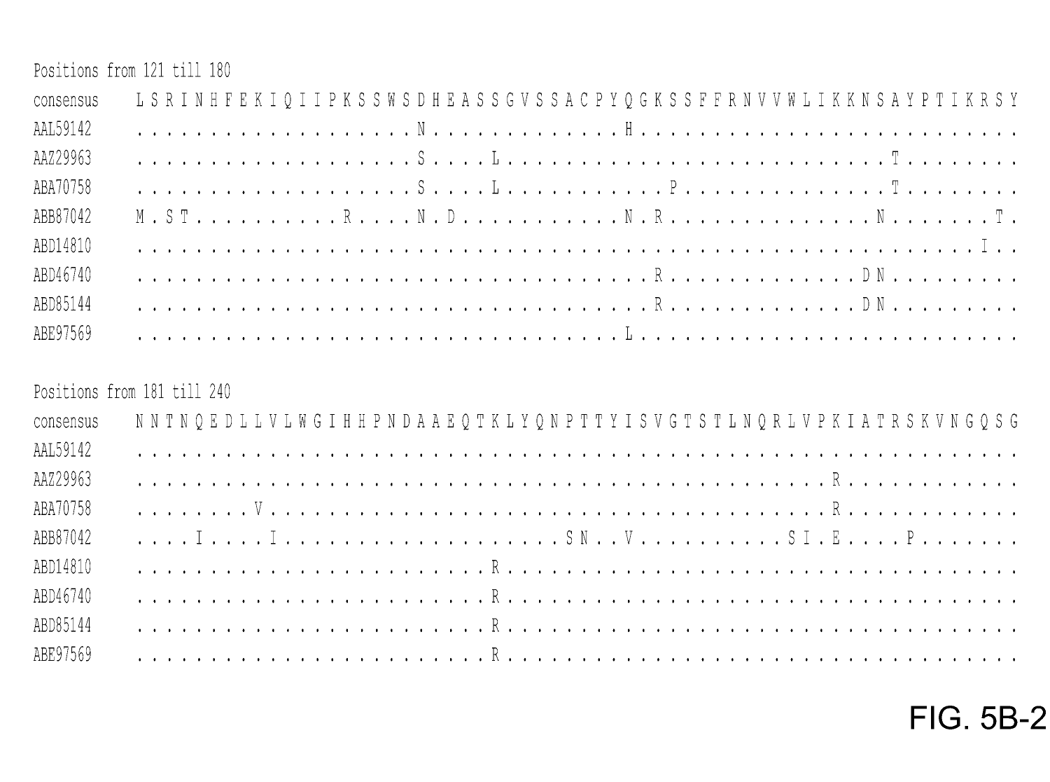

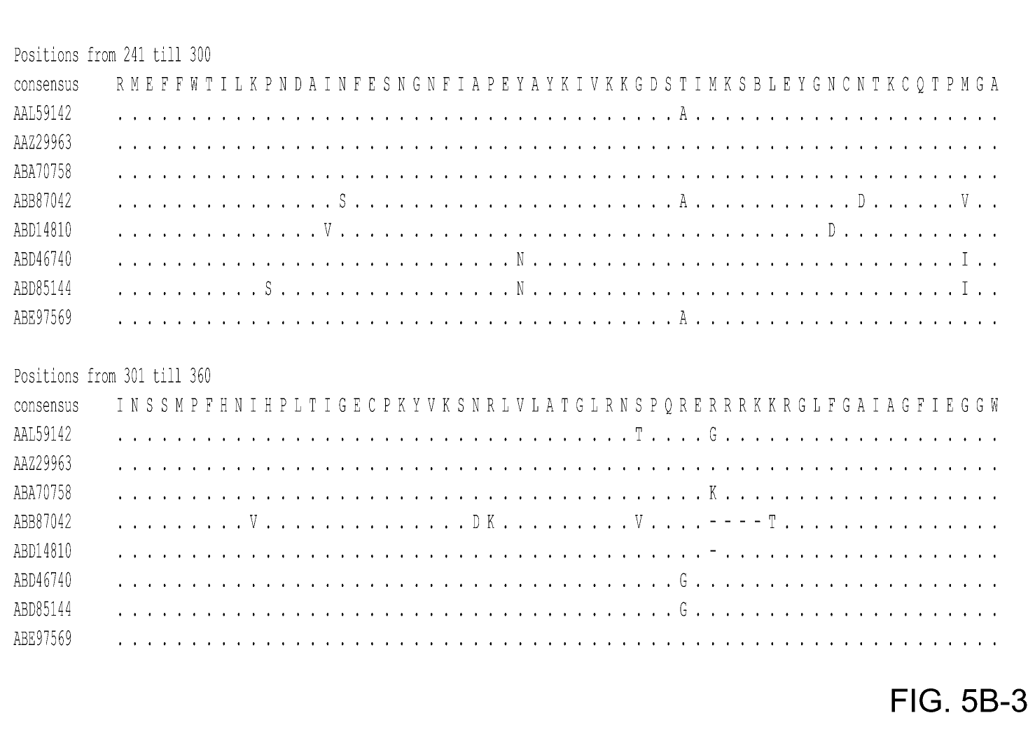

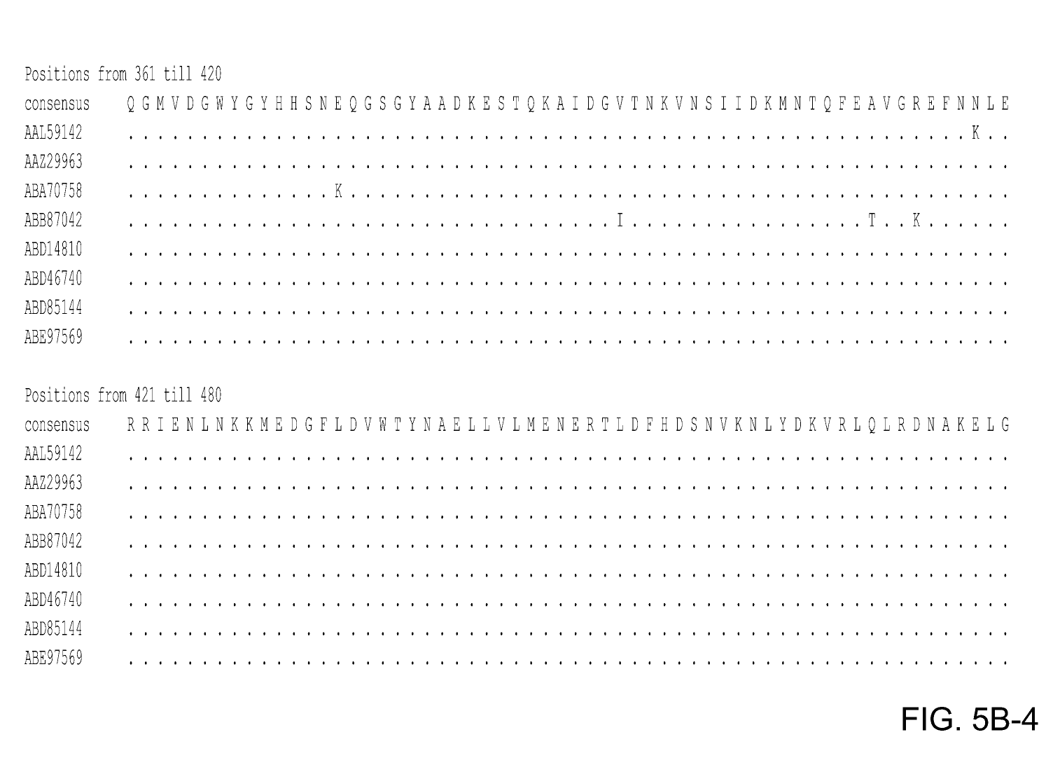

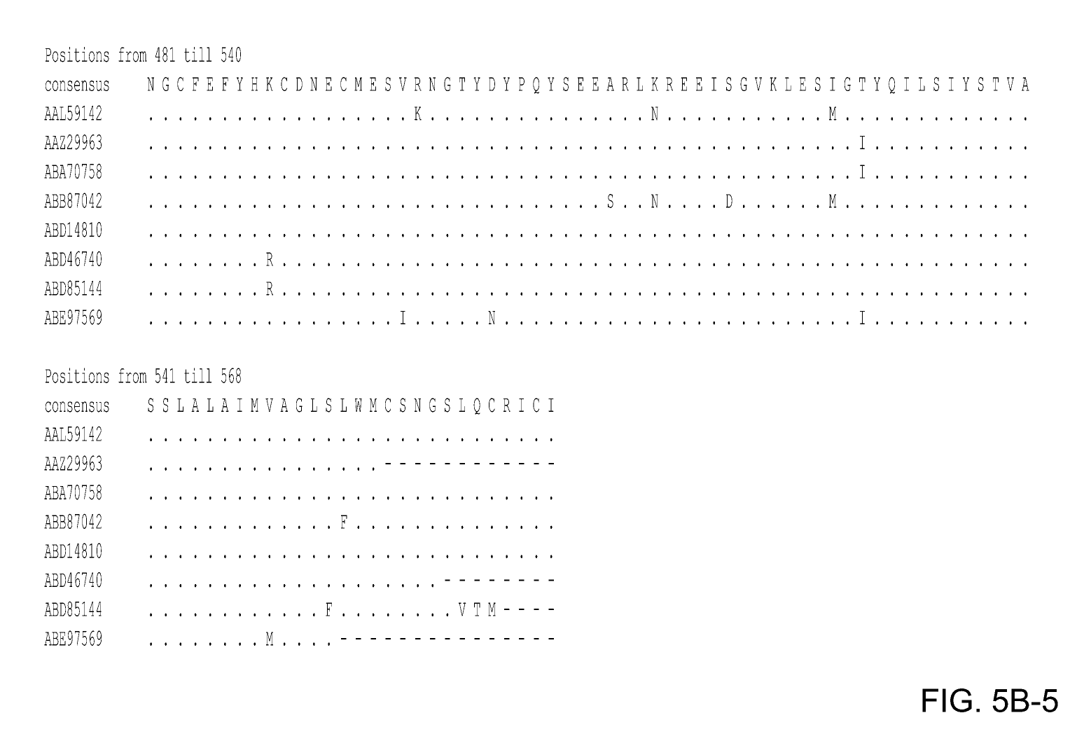

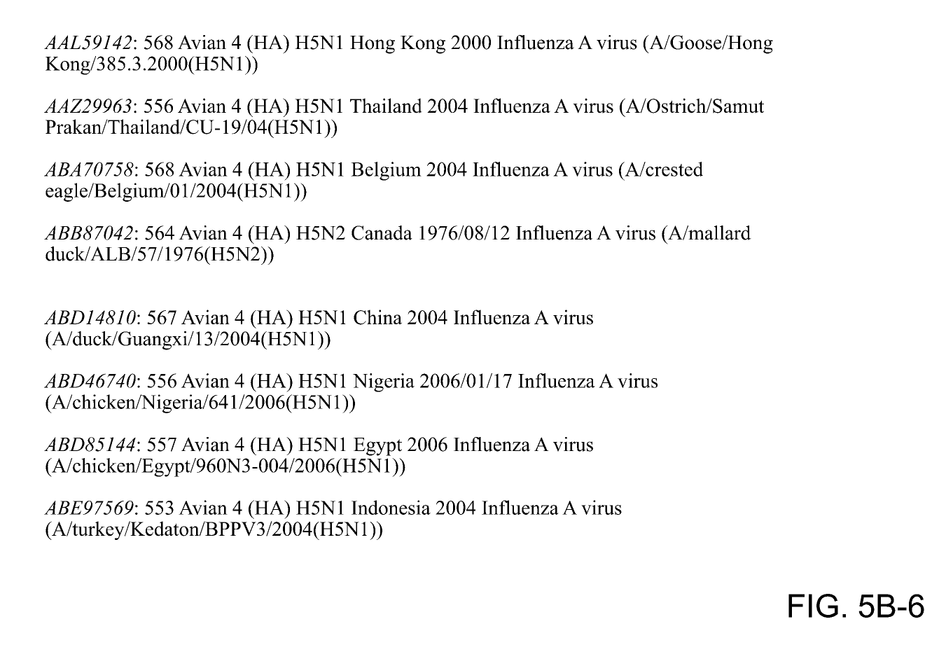

How do the structural variations around the Neu5Acα2-3Gal influence HA-glycan interactions? Lys193, which is highly conserved in the avian H5 (FIG. 5) is positioned to interact with 6-O sulfated Gal and/or 6-O sulfated GlcNAc in Neu5Acα2-3Galβ1-4GlcNAc. This observation is validated by the data mining analysis wherein only the avian H5 binds with high affinity to α2-3 sialylated glycans that are sulfated at the Gal or GlcNAc (Table 3). In a similar fashion, a basic amino acid at position 222 could interact with 4-O sulfated GlcNAc in Neu5Acα2-3Galβ1-3GlcNAc motif or 6-O sulfated GlcNAc in Neu5Acα2-3Galβ1-4GlcNAc motif. On the other hand, a bulky side chain such as Lys222 in H1 and H5 and Trp222 in H3 potentially interferes with a fucosylated GlcNAc in Neu5Acα2-3Galβ1-4(Fucα1-3) GlcNAc motif. This structural observation corroborates the classifier rule α2-3 Type C observed for avian H3 and H5 strains (Table 3), which shows that fucosylation at the GlcNAc is detrimental to binding. The binding of Viet04_H5 HA to α2-3 sialylated glycans is similar to that of ADS97_H5 HA (Table 3) given the almost identical amino acids in their respective glycan binding sites.

Thus, for binding to α2-3 sialylated glycans, apart from the residues that anchor Neu5Ac, Glu190 and Gln226, highly conserved in all avian H1, H3 and H5 subtypes are critical for binding to Neu5Acα2-3Gal motif. The contacts with GlcNAc or GalNAc and substitutions such as sulfation and fucosylation in the α2-3 motif involve amino acids at positions 137, 186, 187, 193 and 222. HA from H1, H3 and H5 exhibit differential binding specificity to the diverse α2-3 sialylated glycans present in the glycan microarray. The amino acid residues in these positions are not conserved across the different HAs and this accounts for the different binding specificities

### Structural Constraints Imposed by Glycan Topology and Substitutions on H1 and H3 HA Binding to α2-6 Sialylated Glycans

In the case of Neu5Aca2-6Gal linkage, the presence of the additional C6-C5 bond provides added conformational flexibility. The position of Gal and subsequent sugars in α2-6 would span a much larger umbrella-like region as compared to the cone-like region in the case of α2-3 (FIG. 6). The binding to α2-6 would involve optimal contacts with the Neu5Ac and Gal sugars at the base of such an umbrella topology and also the subsequent sugars depending on the length of the oligosaccharide. Short α2-6 oligosaccharides such as Neu5Acα2-6Galα1-3/4Glc would potentially adopt a cone-like topology. On the other hand, the presence of a GlcNAc instead of Glc in the α2-6 motif Neu5Acα2-6Galβ1-4GlcNAc- would potentially favor the umbrella topology which is stabilized by optimal van der Waals contact between the acetyl carbons of both GlcNAc and Neu5Ac. However, the α2-6 motif can also adopt a cone topology such that additional factors such as branching and HA binding can compensate for the stability provided by the umbrella topology. The cone topology of the α2-6 motif present as a part of multiple short oligosaccharide branches in an N-linked glycan could be stabilized by intra sugar interactions. On the other hand, the umbrella topology would be favored by the α2-6 motif in a long oligosaccharide branch (at least a tetrasaccharide). The co-crystal structures of H1 and H3 HAs with the α2-6 motif (Neu5Acα2-6Galβ1-4GlcNAc-) motif supports the above notion wherein the φ˜−60 (referred to as cis conformation) causes the sugars beyond Neu5Acα2-6Gal to bend towards the HA protein to make optimal contacts with the binding site (FIG. 7).

In H1 HA, superimposition of the glycan binding domain of HA from a human H1N1 (A/South Carolina/1/1918) subtype with that of ASI30_H1_{—}26 and APR34_H1_{—}26 provided insights into the amino acids involved in providing specificity to the α2-6 sialylated glycan. Lys222 and Asp225 are positioned to interact with the oxygen atoms of the Gal in the Neu5Acα2-6Gal motif. Asp190 and Ser/Asn193 are positioned to interact with additional monosaccharides GlcNAcα1-3Gal of the Neu5Acα2-6Galα1-4GlcNAcα1-3Gal motif (FIG. 15, Panels A,B).

Asp190, Lys222 and Asp225 are highly conserved among the H1 HAs from the 1918 human pandemic strains. Although the amino acid Gln226 is highly conserved in all the avian and human H1 subtypes, it does not appear to be as involved in binding to α2-6 sialylated glycans (in human H1 subtypes) compared to its role in binding to α2-3 sialylated glycans (in the avian H1 subtypes). The data mining analysis of the glycan array results for wild type and mutant form of the avian and human H1 HAs further substantiates the role of the above amino acids in binding to α2-6 sialylated glycans (Table 3). The Glu190Asp/Gly225Asp double mutant of the avian H1 HA reverses its binding to α2-6 sialylated glycans (Table 3). Further, the Lys222Leu mutant of human ANY18_H1 removes its binding to all the sialylated glycans on the array consistent with the essential role of Lys222 in glycan binding.

In order to identify amino acids that provide specificity for H3N2 HA binding to α2-6 sialylated glycans, the glycan binding domain of HA from human H3N2 (AAI68_H3), ADU63_H3_{—}26 and ASI30_H1_{—}26 were superimposed. Analysis of these superimposed structures showed that Leu226 is positioned to provide optimal van der Waals contact with the C6 atom of the Neu5α2-6Gal motif and Ser228 is positioned to interact with O9 of the sialic acid. Ser228 in the human H3 also interacts with Glu190 (unlike Gly228 in avian ADU63_H3 which does not) thereby affecting its side chain conformation. The side chain of Glu190 in human H3 HA is displaced slightly into the binding site by about 0.7 Å in comparison with that of Glu190 in avian H3 HA. These differences limit the ability of human H3 HA to bind to α2-3 sialylated glycans and correlate with its preferential binding to α2-6 sialylated glycans. Thus, the Gln226Leu and Gly228Ser mutations cause a reversal of the glycan receptor specificity of avian H3 to human H3 subtype during the 1967 pandemic.

Comparison of HAs from 1967-68 pandemic H3N2 and those from more recent H3 subtypes (after 1990) show that the Glu190 is mutated to Asp in the recent subtypes. This mutation further enhances the binding of human H3 to α2-6 sialylated glycans since Asp190 in human H3 is positioned to interact favorably with these glycans. This structural implication is further corroborated by the data mining analysis of the glycan array data on a human H3 subtype (A/Moscow/10/1999). This HA comprises Asp190, Leu226 and Ser228 (FIG. 2) and shows strong preference to α2-6 sialylated glycans (Table 3).

The above observations highlight both the similarities as well as differences between H1 and H3 HA binding to α2-6 sialylated glycans. In both H1 and H3 HA, Asp190 and Ser/Asn193 are positioned to make favorable contacts with monosaccharides beyond Neu5Acα2-6Gal motif (FIG. 15, Panels A,B). The differences in the amino acids and their contacts with α2-6 sialylated glycans between H1 and H3 HA provide distinct surface and ionic complimentarity for binding these glycans. The Neu5Acα2-6Gal linkage has an additional degree of conformational freedom than the Neu5Acα2-3Gal. Thus the HA binding to α2-6 sialylated glycans has a more open binding pocket to accommodate this conformational freedom. While Leu226 in human H3 HA is positioned to provide optimal van der Waals contact with Neu5Acα2-6Gal, the ionic contacts provided by Gln226 in H1 HA to this motif are not as optimal. On the other hand in H1, the amino acids Lys222 and Asp225 provide more optimal ionic contacts with α2-6 sialylated glycans compared to Trp222 and Gly225 in H3.

### Structural Constraints for Binding of Wild Type and Mutant H5 HAs to α2-6 Sialylated Glycans

The interactions with α2-6 sialylated glycans provided by the different amino acids in H1 and H3 HA suggested that the current avian H5N1 HA could mutate into a H1-like or H3-like glycan binding site in order to reverse its glycan receptor specificity. Based on the above framework, the hypothesized H1-like and H3-like mutations for H5 HA are further elaborated and tested as discussed below.

Analysis of the superimposed ASI30_H1_{—}26, APR34_H1_{—}26, ADS97_H5_{—}26 and Viet04_H5 structures provided insights into the H1-like binding of H5 HA to α2-6 sialylated glycans. Since the H1 and H5 HAs belong to the same structural clade, their glycan binding sites share a similar topology and distribution of amino acids (Russell et al., Virology, 325:287, 2004). Lys222, which is highly conserved in avian H5 HAs is positioned to provide optimal contacts with Gal of Neu5Acα2-6Gal motif similar to the analogous Lys in H1 HA. Glu190 and Gly225 in Viet04_H5 (in the place of Asp190 and Asp225 in H1) do not provide the necessary contacts with the Neu5Acα2-6Galβ1-4GlcNAc motif similar to H1. Therefore Glu190Asp and Gly225Asp mutations in H5 HA could potentially improve the contacts with α2-6 sialylated glycans.

Analysis of the interactions beyond GlcNAc in the Neu5Acα2-6Galβ1-4GlcNAcβ1-3Galβ1-4Glc oligosaccharide and the glycan binding pocket of H1 and H5 HAs showed that while Ser/Asn193 in H1 HA provides favorable contacts with the penultimate Gal, the analogous Lys193 in H5 has unfavorable steric overlaps with the GlcNAcβ1-3Gal motif. Thus, the Lys193Ser mutation can provide additional favorable contacts (along with Glu190Asp and Gly225Asp mutations) with α2-6 sialylated glycans.

The highly conserved Gln226 in H1 HA is also conserved in the avian H5 HA. Given that Gln226 plays a less active role in H1 HA binding to α2-6 sialylated glycans (as discussed above), mutation of this amino acid to a hydrophobic amino acid such as Leu could potentially enhance its van der Waals contact with C6 atom of Gal in Neu5Acα2-6Gal motif.

The superimposition of ADU63_H3_{—}26, AAI68_H3, ADS97_H5_{—}26 and Viet04_H5 provides insights into the H3-like binding of H5 HA to α2-6 sialylated glycans. While this superimposition structurally aligned the glycan binding site of H5 and H3 HA, it was not as good as the structural alignment between H5 and H1. The favorable van der Waals contact and ionic contact with Neu5α2-6Gal motif respectively provided by Leu226 and Ser228 in H3 HA were absent in H5 HA (with Gln226 and Gly228). Given that Leu226 and Ser228 are critical for binding to α2-6 sialylated glycans in human H3 HA, the Gln226Leu and Gly228Ser mutations in H5 HA could potentially provide optimal contacts with α2-6 sialylated glycans. Further, even in the comparison between H3 and H5, Lys 193 is positioned such that it would have unfavorable steric contacts with the monosaccharides beyond Neu5Acα2-6Gal motif as against Ser193 in human H3 HA which is positioned to provide favorable contacts. Although the HA from the 1967-68 pandemic H3N2 comprises of Glu190, Asp190 in H5 HA would be positioned to provide better ionic contacts with Neu5Acα2-6Gal motif in longer oligosaccharides.

The roles of the above mentioned residues were further corroborated by data mining analysis of glycan array data for wild type and mutant forms of Viet04_H5 (Table 3). The double mutant, Glu190Asp/Gly225Asp, does not bind to any glycan structure since it loses the amino acid Glu190 for binding α2-3 sialylated glycans and has the steric interference from Lys193 for binding to α2-6 sialylated glycans. Similarly the double mutant, Gln226Leu/Gly228Ser binds to some of the α2-3 sialylated glycans (α2-3 Type B classifier) but only to a single biantennary α2-6 sialylated glycan (α2-6 Type A classifier).

Analysis of this binding to the biantennary α2-6 sialylated glycan showed that the Neu5Acα2-6Gal linkage in this glycan can potentially bind in an extended conformation to the double mutant albeit with lesser contacts (FIG. 16). Furthermore, the Neu5Acα2-6Gal on the Malα1-3Man branch binds more favorably compared to the same motif on the Manα1-6Man branch which has unfavorable steric contacts with the glycan binding site of H5 HA (FIG. 16). The narrow specificity of the Gln226Leu/Gly228Ser double mutant to α2-6 sialylated glycans is consistent with Lys193 interfering with the binding.

Without wishing to be bound by any particular theory, the present inventors propose that a necessary condition for human adaptation of influenza A virus HAs is to gain the ability to bind to long α2-6 (predominantly expressed in human upper airway) with high affinity. For example, an aspect of glycan diversity is the length of the lactosamine branch that is capped with the sialic acid. This is captured by the two distinct features of α2-6 sialylated glycans derived from the data mining analysis (Table 3). One feature is characterized by the Neu5Acα2-6Galβ1-4GlcNAc linked to the Man of the N-linked core and the other is characterized by this motif linked to another lactose amine unit forming a longer branch (which typically adopts umbrella topology). Thus, the extensive binding of the mutant H5 HAs to the upper airways may only be possible if these mutants bind with high affinity to the glycans with long α2-6 adopting the umbrella topology. For example, according to the present invention, desirable binding patterns include binding to umbrella glycans depicted in FIG. 9.

By contrast, we note a recent report of modified H5 HA proteins (containing Gly228Ser and Gln226Leu/Gly228Ser substitution) showed binding to only a single biantennary a2-6 sualyl-lactosamine glycan structure on the glycan array (Stevens et al., Science 312:404, 2006). Such modified H5 HA proteins are therefore not BSHB H5 HAs, as described herein.

### Example 2

### Cloning, Baculovirus Synthesis, Expression and Purification of HA

Hemagglutinin in viruses is present as a trimer and is anchored to the membrane. The full length construct of HA has a N-terminal signal peptide and a C-terminal transmembrane sequence. For recombinant expression of HA, often a shortened construct of HA is used which allows the protein to be secreted. This shortened soluble construct is created by replacing the HA's N-terminal signal peptide with a Gp67 signal peptide sequence and the C-terminal transmembrane region is replaced by a ‘foldon’ sequence followed by a tryptic cleavage site and a 6×-His tag (Stevens et al., J. Mol. Biol., 355:1143, 2006). Both full length and the soluble form of HA were expressed in insect cells. Suspension cultures of Sf-9 cells in Sf900 II SFM medium (Invitrogen) were infected with baculoviruses containing either full length or soluble form of HA. The cells were harvested 72-96 hours post infection.

Hemagglutinin (HA) from A/Vietnam/1203/2004 was a kind gift from Adolfo García-Sastre. This “wild type” (WT) HA was used as template to create two different mutant constructs, DSLS and DSDL, using QuikChange II XL Site-Directed Mutagenesis Kit (Stratagene) and QuikChange Multi Site-Directed Mutagenesis Kit (Stratagene). The primers used for mutagenesis were designed using the web based program, PrimerX (http://bioinformatics.org/primerx/), and synthesized by Invitrogen. The WT and mutant HA genes were sub-cloned into the entry vector pENTR-D-TOPO (Invitrogen) using TOPO ligation. The entry vectors containing the WT and mutant genes were recombined with BaculoDirect linear DNA (Invitrogen) using Gateway cloning technology. DNA sequencing was performed at each sub-cloning step to confirm the accuracy of the sequences. The recombinant baculovirus DNA produced was used to transfect Spodoptera frugiperda Sf-9 cells (Invitrogen) to yield primary stock of virus.

The full length HA was purified from the membrane fraction of the infected cells by a method modified from Wang et al. (2006) Vaccine, 24:2176. Briefly, the cells from the 150 ml culture were harvested by centrifugation and the cell pellet was extracted with 30 ml of 1% Tergitol NP-9 in buffer A (20 mM sodium phosphate, 1.0 mM EDTA, 0.01% Tergitol-NP9, 5% glycerol, pH 5.93) at 4° C. for 30 min. The extract was then subjected to centrifugation at 6,000 g for 15 min. The supernatant was filtered using a 0.45 micron filter and loaded on Q/SP columns (GE healthcare, Piscataway, N.J.) that were previously equilibrated with Buffer A. After loading, the columns were washed with 20 ml of Buffer A. Then, the anion exchange column Q was disconnected and the SP column was used for elution of protein using five 5 ml fractions of buffer B (20 mM sodium phosphate, 0.03% Tergitol, 5% glycerol, pH 8.2) and two 5 ml fractions of buffer C (20 mM sodium phosphate, 150 mM NaCl, 0.03% Tergitol, 5% glycerol, pH 8.2). The fractions containing the protein of interest were pooled together and subjected to ultrafiltration using Amicon Ultra 100 K NMWL membrane filters (Millipore). The protein was concentrated and reconstituted in PBS.

The soluble form of HA was purified from the supernatant of the infected cells using the protocol described in Stevens et al. (2004). Briefly, the supernatant was concentrated and the soluble HA was recovered from the concentrated cell supernatant by performing affinity chromatography using Ni-NTA beads (Qiagen). Eluting fractions containing HA were pooled and dialyzed against 10 mM Tris-HCl, 50 mM NaCl; pH 8.0. Ion exchange chromatography was performed on the dialyzed samples using a Mono-Q HR10/10 column (Pharmacia). The fractions containing HA were pooled together and subjected to ultrafiltration using Amicon Ultra 100 K NMWL membrane filters (Millipore). The protein was concentrated and reconstituted in PBS.

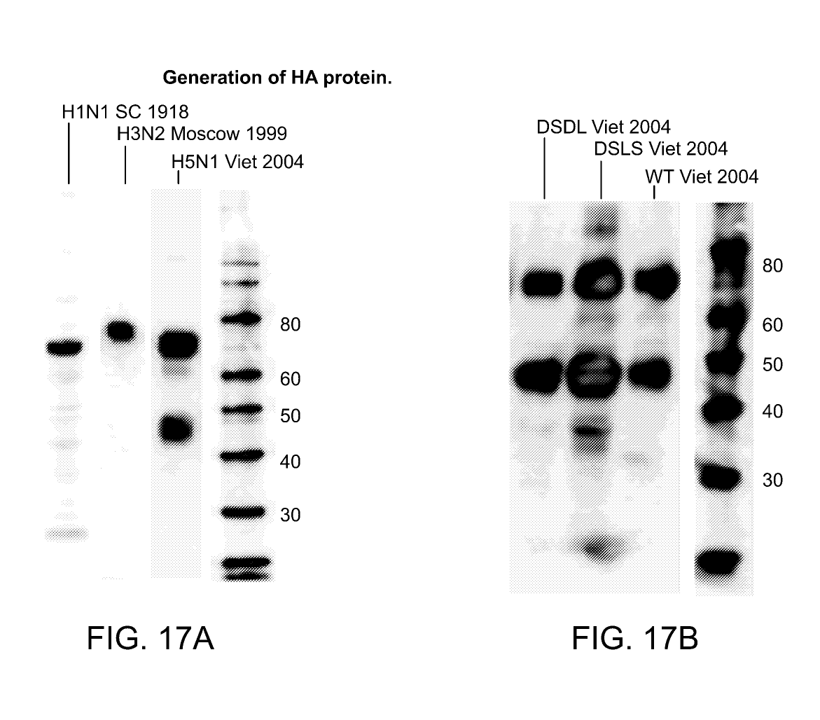

The presence of the protein in the samples was verified by performing western blot analysis with anti avian H5N1 HA antibody. Through dot-blot immunoassay (using WT H5 HA obtained from Protein Sciences Inc as the reference) the protein concentration of WT and the mutants were determined. In the various experiments that were performed the protein concentration of the H5 HA (WT and mutants) were typically found to be in 20-50 microgram/ml range. Based on the protein concentration for a given lot appropriate serial dilutions in the ranges of 1:10-1:100 were used (see FIG. 17).

### Example 3

### Application of Data Mining Platform to Investigate Glycan Binding Specificity of HA

A framework for the binding of H5N1 subtype to α2-3/6 sialylated glycans was developed (FIG. 7). This framework comprises two complementary analyses. The first involves a systematic analysis of an HA glycan binding site and its interactions with α2-3 and α2-6 sialylated glycans using the H1, H3 and H5 HA-glycan co-crystal structures (Table 2).

This analysis provides important insights into the interactions of an HA glycan binding site with a variety of α2-3/6 sialylated glycans, including glycans of either umbrella or cone topology. The second involves a data mining approach to analyze the glycan array data on the different H1, H3 and H5 HAs. This data mining analysis correlates the strong, weak and non-binders of the different wild type and mutant HAs to the structural features of the glycans in the microarray (Table 3).

Importantly, these correlations (classifiers) capture the effect of subtle structural variations of the α2-3/6 sialylated linkages and/or of different topologies on binding to the different HAs. The correlations of glycan features obtained from the data mining analysis are mapped onto the HA glycan binding site, providing a framework to systematically investigate the binding of H1, H3 and H5 HAs to α2-3 and α2-6 sialylated glycans, including glycans of different topologies, as discussed below.

To give but one example, application of this framework to H5 HA according to the present invention illustrates how length of an α2-6 oligosaccharide chain becomes more important, especially in the context of degree of branching, than the nuances of structural variations around the glycan. For example, a triantennary structure with a single α2-6 motif versus a biantennary structure with a longer α2-6 motif will influence HA-glycan binding as against structural variations around the individual α2-6 motif. This is confirmed by the distinct length dependent classifiers for the α2-6 motif obtained herein from data mining (Table 3).

### Example 4

### Broad Spectrum Human Binding H5 HA Polypeptides

In some particular embodiments of the present invention, HA polypeptides are H5 polypeptides. In some such embodiments, inventive H5 polypeptides show binding (e.g., high affinity and/or specificity binding) to umbrella glycans. In some embodiments, inventive H5 polypeptides are termed “broad spectrum human binding” (BSHB) H5 polypeptides.

The phrase “broad spectrum human binding” (BSHB) was originally coined to refer to H5 polypeptides bind to HA receptors found in human epithelial tissues, and particularly to human HA receptors characterized by α2-6 sialylated glycans. As discussed above, with regard to HA polypeptides generally, in some embodiments, inventive BSHB H5 HA polypeptides bind to receptors found on human upper respiratory epithelial cells. Furthermore, inventive BSHB H5 HA polypeptides bind to a plurality of different α2-6 sialylated glycans. In certain embodiments, BSHB H5 HA polypeptides bind to umbrella glycans.

In certain embodiments, inventive BSHB H5 HA polypeptides bind to HA receptors in the bronchus and/or trachea. In some embodiments, BSHB H5 HA polypeptides are not able to bind receptors in the deep lung, and in other embodiments, BSHB H5 HA polypeptides are able to bind receptors in the deep lung. In further embodiments, BSHB H5 HA polypeptides are not able to bind to α2-3 sialylated glycans, and in other embodiments BSHB H5 HA polypeptides are able to bind to α2-3 sialylated glycans.

In certain embodiments, inventive BSHB H5 HA polypeptides are variants of a parent H5 HA (e.g., an H5 HA found in a natural influenza isolate). For example, in some embodiments, inventive BSHB H5 HA polypeptides have at least one amino acid substitution, as compared with wild type H5 HA, within or affecting the glycan binding site. In some embodiments, such substitutions are of amino acids that interact directly with bound glycan; in other embodiments, such substitutions are of amino acids that are one degree of separation removed from those that interact with bound glycan, in that the one degree of separation removed-amino acids either (1) interact with the direct-binding amino acids; (2) otherwise affect the ability of the direct-binding amino acids to interact with glycan, but do not interact directly with glycan themselves; or (3) otherwise affect the ability of the direct-binding amino acids to interact with glycan, and also interact directly with glycan themselves. Inventive BSHB H5 HA polypeptides contain substitutions of one or more direct-binding amino acids, one or more first degree of separation-amino acids, one or more second degree of separation-amino acids, or any combination of these. In some embodiments, inventive BSHB H5 HA polypeptides may contain substitutions of one or more amino acids with even higher degrees of separation.

In certain embodiments, inventive BSHB H5 HA polypeptides have at least two, three, four, five or more amino acid substitutions as compared with wild type H5 HA; in some embodiments inventive BSHB H5 HA polypeptides have two, three, or four amino acid substitutions. In some embodiments, all such amino acid substitutions are located within the glycan binding site.

In certain embodiments, a BSHB H5 HA polypeptide has one or more amino acid substitutions relative to wild type H5 HA at residues selected from the group consisting of residues 98, 136, 138, 153, 155, 159, 183, 186, 187, 190, 193, 194, 195, 222, 225, 226, 227, and 228. In other embodiments, a BSHB H5 HA polypeptide has one or more amino acid substitutions relative to wild type H5 HA at residues selected from amino acids located in the region of the receptor that directly binds to the glycan, including but not limited to residues 98, 136, 153, 155, 183, 190, 193, 194, 222, 225, 226, 227, and 228. In further embodiments, a BSHB H5 HA polypeptide has one or more amino acid substitutions relative to wild type H5 HA at residues selected from amino acids located adjacent to the region of the receptor that directly binds the glycan, including but not limited to residues 98, 138, 186, 187, 195, and 228.

In further embodiments, a BSHB H5 HA polypeptide has one or more amino acid substitutions relative to wild type H5 HA at residues selected from the group consisting of residues 138, 186, 187, 190, 193, 222, 225, 226, 227 and 228. In other embodiments, a BSHB H5 HA polypeptide has one or more amino acid substitutions relative to wild type H5 HA at residues selected from amino acids located in the region of the receptor that directly binds to the glycan, including but not limited to residues 190, 193, 222, 225, 226, 227, and 228. In further embodiments, a BSHB H5 HA polypeptide has one or more amino acid substitutions relative to wild type H5 HA at residues selected from amino acids located adjacent to the region of the receptor that directly binds the glycan, including but not limited to residues 138, 186, 187, and 228.

In further embodiments, a BSHB H5 HA polypeptide has one or more amino acid substitutions relative to wild type H5 HA at residues selected from the group consisting of residues 98, 136, 153, 155, 183, 194, and 195. In other embodiments, a BSHB H5 HA polypeptide has one or more amino acid substitutions relative to wild type H5 HA at residues selected from amino acids located in the region of the receptor that directly binds to the glycan, including but not limited to residues 98, 136, 153, 155, 183, and 194. In further embodiments, a BSHB H5 HA polypeptide has one or more amino acid substitutions relative to wild type H5 HA at residues selected from amino acids located adjacent to the region of the receptor that directly binds the glycan, including but not limited to residues 98 and 195.

In certain embodiments, a BSHB H5 HA polypeptide has one or more amino acid substitutions relative to wild type H5 HA at residues selected from amino acids that are one degree of separation removed from those that interact with bound glycan, in that the one degree of separation removed-amino acids either (1) interact with the direct-binding amino acids; (2) otherwise affect the ability of the direct-binding amino acids to interact with glycan, but do not interact directly with glycan themselves; or (3) otherwise affect the ability of the direct-binding amino acids to interact with glycan, and also interact directly with glycan themselves, including but not limited to residues 98, 138, 186, 187, 195, and 228.

In further embodiments, a BSHB H5 HA polypeptide has one or more amino acid substitutions relative to wild type H5 HA at residues selected from amino acids that are one degree of separation removed from those that interact with bound glycan, in that the one degree of separation removed-amino acids either (1) interact with the direct-binding amino acids; (2) otherwise affect the ability of the direct-binding amino acids to interact with glycan, but do not interact directly with glycan themselves; or (3) otherwise affect the ability of the direct-binding amino acids to interact with glycan, and also interact directly with glycan themselves, including but not limited to residues 138, 186, 187, and 228.

In further embodiments, a BSHB H5 HA polypeptide has one or more amino acid substitutions relative to wild type H5 HA at residues selected from amino acids that are one degree of separation removed from those that interact with bound glycan, in that the one degree of separation removed-amino acids either (1) interact with the direct-binding amino acids; (2) otherwise affect the ability of the direct-binding amino acids to interact with glycan, but do not interact directly with glycan themselves; or (3) otherwise affect the ability of the direct-binding amino acids to interact with glycan, and also interact directly with glycan themselves, including but not limited to residues 98 and 195.

In certain embodiments, a BSHB H5 HA polypeptide has an amino acid substitution relative to wild type H5 HA at residue 159.

In other embodiments, a BSHB H5 HA polypeptide has one or more amino acid substitutions relative to wild type H5 HA at residues selected from 190, 193, 225, and 226. In some embodiments, a BSHB H5 HA polypeptide has one or more amino acid substitutions relative to wild type H5 HA at residues selected from 190, 193, 226, and 228. In some embodiments, an inventive HA polypeptide variant, and particularly an H5 variant has one or more of the following amino acid substitutions: Ser137Ala, Lys156Glu, Asn186Pro, Asp187Ser, Asp187Thr, Ala189Gln, Ala189Lys, Ala189Thr, Glu190Asp, Glu190Thr, Lys193Arg, Lys193Asn, Lys193His, Lys193Ser, Gly225Asp, Gln226Ile, Gln226Leu, Gln226Val, Ser227Ala, Gly228Ser.

In some embodiments, an inventive HA polypeptide variant, and particularly an H5 variant has one or more of the following sets of amino acid substitutions:

Glu190Asp, Lys193Ser, Gly225Asp and Gln226Leu;

Glu190Asp, Lys193Ser, Gln226Leu and Gly228Ser;

Ala189Gln, Lys 193 Ser, Gln226Leu, Gly228Ser;

Ala189Gln, Lys193Ser, Gln226Leu, Gly228Ser;

Asp187Ser/Thr, Ala189Gln, Lys193Ser, Gln226Leu, Gly228Ser;

Ala189Lys, Lys193Asn, Gln226Leu, Gly228Ser;

Asp187Ser/Thr, Ala189Lys, Lys193Asn, Gln226Leu, Gly228Ser;

Lys156Glu, Ala189Lys, Lys193Asn, Gln226Leu, Gly228Ser;

Lys193His, Gln226Leu/Ile/Val, Gly228Ser;

Lys193Arg, Gln226Leu/Ile/Val, Gly228Ser;

Ala189Lys, Lys193Asn, Gly225Asp;

Lys156Glu, Ala189Lys, Lys193Asn, Gly225Asp;

Ser137Ala, Lys156Glu, Ala189Lys, Lys193Asn, Gly225Asp;

Glu190Thr, Lys193Ser, Gly225Asp;

Asp187Thr, Ala189Thr, Glu190Asp, Lys193Ser, Gly225Asp;

Asn186Pro, Asp187Thr, Ala189Thr, Glu190Asp, Lys193Ser, Gly225Asp;

Asn186Pro, Asp187Thr, Ala189Thr, Glu190Asp, Lys193Ser, Gly225Asp, Ser227Ala.

In some such embodiments, the HA polypeptide has at least one further substitution as compared with a wild type HA, such that affinity and/or specificity of the variant for umbrella glycans is increased.

In certain embodiments, inventive BSHB H5 HA polypeptides have amino acid sequences characteristic of H1 HAs. For example, in some embodiments, such H1-like BSHB H5 HA polypeptides have substitutions Glu190Asp, Lys193Ser, Gly225Asp and Gln226Leu.

In certain embodiments, inventive BSHB H5 HA polypeptides have amino acid sequences characteristic of H1 HAs. For example, in some embodiments, such H3-like BSHB H5 HAs contain substitutions Glu190Asp, Lys193Ser, Gln226Leu and Gly228Ser.

In some embodiments, inventive BSHB H5 HA polypeptides have an open binding site as compared with wild type H5 HAs. In some embodiments, inventive BSHB H5 HA polypeptides bind to the following α2-6 sialylated glycans:

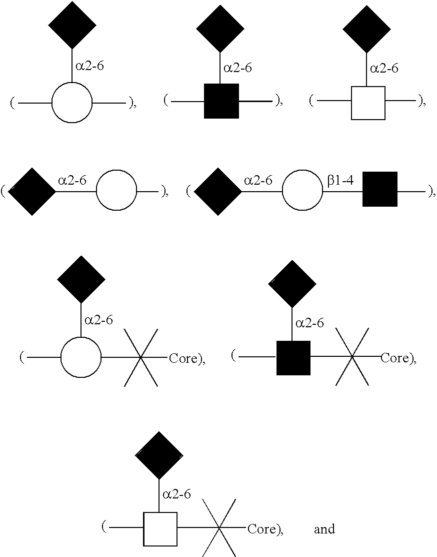

combinations thereof. In some embodiments, inventive BSHB H5 HA polypeptides bind to glycans of the structure:

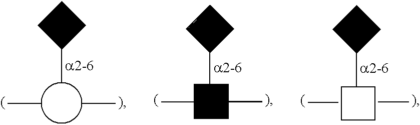

and combinations thereof; and/or

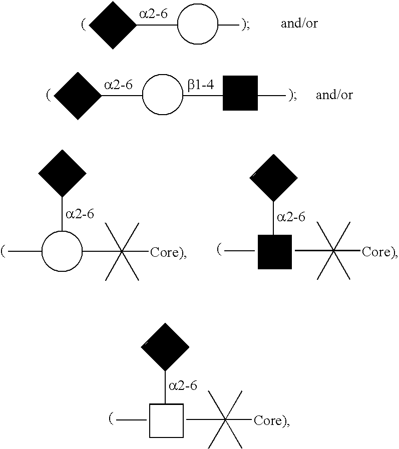

and combinations thereof. In some embodiments, inventive BSHB H5 HA polypeptides bind to

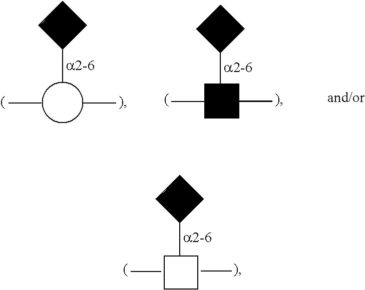

in some embodiments to

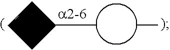

in some embodiments to

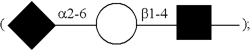

and in some embodiments to

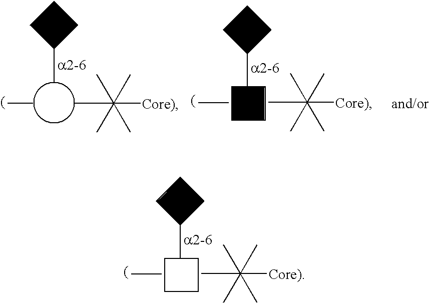

In some embodiments, inventive BSHB H5 HA polypeptides bind to umbrella topology glycans. In some embodiments, inventive BSHB H5 HA polypeptides bind to at least some of the glycans (e.g., α2-6 silaylated glycans) depicted in FIG. 9. In some embodiments, inventive BSHB H5 HA polypeptides bind to multiple glycans depicted in FIG. 9.

In some embodiments, inventive BSHB H5 HA polypeptides bind to at least about 10%, 15%, 20%, 25%, 30% 35%, 40%, 45%, 50%, 55%, 60%, 65%, 70%, 75%, 80%, 85%, 90% 95% or more of the glycans found on HA receptors in human upper respiratory tract tissues (e.g., epithelial cells).

### Example 5

### Glycan Diversity in the Human Upper Respiratory Tissues

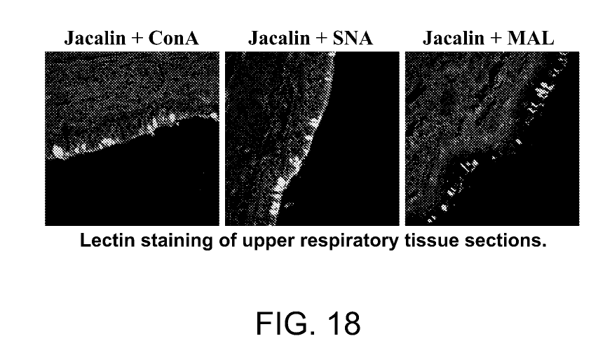

Lectin binding studies showed diversity in the distribution of α2-3 and α2-6 in the upper respiratory tissues. Staining studies indicate predominant distribution of α2-6 sialylated glycans as a part of both N-linked (ciliated cells) and O-linked glycans (in the goblet cells) on the apical side of the tracheal epithelium (FIG. 18). On the other hand, the internal regions of the tracheal tissue predominantly comprises of α2-3 distributed on N-linked glycans. A long-standing question is what α2-6 sialylated glycan receptors are present on human lungs?

MALDI-MS glycan profiling analyses showed a substantial diversity (FIG. 10) as well as predominant expression of α2-6 sialylated glycans on the human upper airways. Significantly, fragmentation of representative mass peaks using MALDI TOF-TOF supports glycan topologies where longer oligosaccharide branches with multiple lactosamine repeats are extensively distributed as compared to short oligosaccharide branches (FIG. 10). To provide a reference for the diversity in the distribution and topology of glycans in the upper airway, MALDI-MS analysis was performed on N-linked glycans from human colonic epithelial cells (HT29). It is known that the current H5N1 viruses primarily infect the gut and hence these cells were chosen as representative gut cells. The glycan profile of HT29 cells is significantly different from that of the HBEs wherein there is a predominant distribution of α2-3 and the long oligosaccharide branch glycan topology is not as observed (FIG. 10).

Data in FIG. 18 were generated by the following method. Formalin fixed and paraffin embedded human tracheal tissue sections were purchased from US Biological. After the tissue sections were deparaffinized and rehydrated, endogenous biotin was blocked using the streptavidin/biotin blocking kit (Vector Labs). Sections were then incubated with FITC labeled Jacalin (specific for O-linked glycans), biotinylated Concanavalin A (Con A, specific for α-linked mannose residues, which are part of the core oligosaccharide structure that constitute N-linked glycans), biotinylated Maackia amurensis lectin (MAL, specific for SAα2,3-gal) and biotinylated Sambuccus nigra agglutinin (SNA, specific for SAα2,6-gal) (Vector labs; 10 μg/ml in PBS with 0.5% Tween-20) for 3 hrs. After washing with TBST (Tris buffered saline with 1% Tween-20), the sections were incubated with Alexa fluor 546 streptavidin (2 μg/ml in PBS with 0.5% Tween-20) for 1 hr. Slides were washed with TBST and viewed under a confocal microscope (Zeiss LSM510 laser scanning confocal microscopy). All incubations were performed at room temperature (RT).

Data in FIG. 10 were generated using the following method. The cells (˜70×10⁶) were harvested when they were >90% confluent with 100 mM citrate saline buffer and the cell membrane was isolated after treatment with protease inhibitor (Calbiochem) and homogenization. The cell membrane fraction was treated with PNGaseF (New England Biolabs) and the reaction mixture was incubated overnight at 37° C. The reaction mixture was boiled for 10 min to deactivate the enzyme and the deglycosylated peptides and proteins were removed using a Sep-Pak C18 SPE cartridge (Waters). The glycans were further desalted and purified into neutral (25% acetonitrile fraction) and acidic (50% acetonitrile containing 0.05% trifluoroacetic acid) fractions using graphitized carbon solid-phase extraction columns (Supelco). The acidic fractions were analyzed by MALDI-TOF MS in positive and negative modes respectively with soft ionization conditions (accelerating voltage 22 kV, grid voltage 93%, guide wire 0.3% and extraction delay time of 150 ns). The peaks were calibrated as non-sodiated species. The predominant expression of α2-6 sialylated glycans was confirmed by pretreatment of samples using Sialidase A and S. The isolated glycans were subsequently incubated with 0.1 U of Arthrobacter ureafaciens sialidase (Sialidase A, non-specific) or Streptococcus pneumoniae sialidase (Sialidase S, specific for α2-3 sialylated glycans) in a final volume of 100 mL of 50 mM sodium phosphate, pH 6.0 at 37° C. for 24 hrs. The neutral and the acidic fractions were analyzed by MALDI-TOF MS in positive and negative modes respectively.

### Example 6

### Dose Response Binding of H1 and H3 HA to Human Lung Tissues

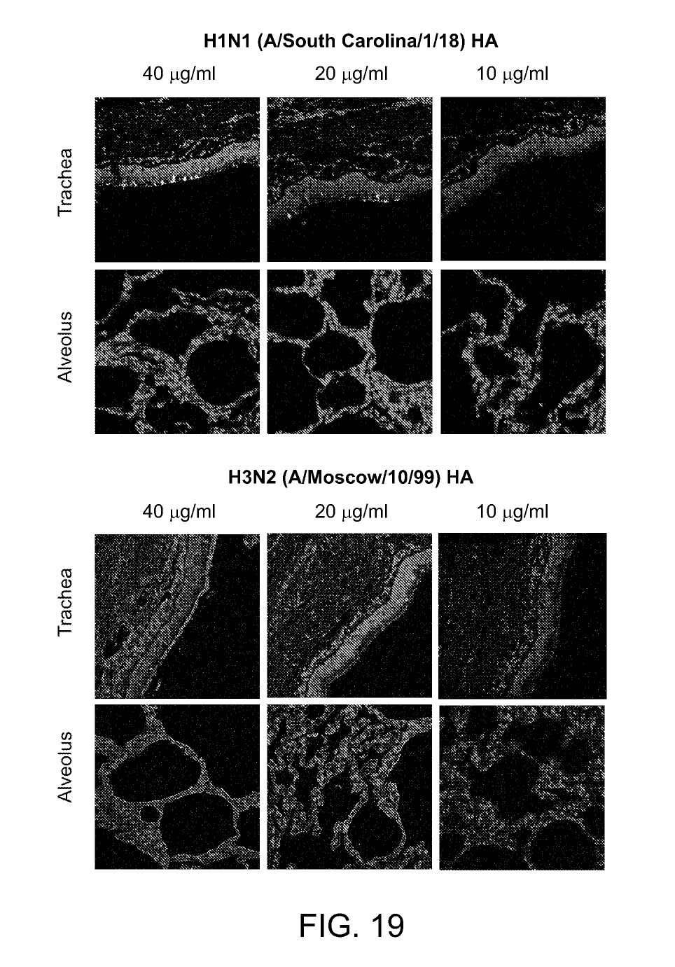

The apical side of tracheal tissue predominantly expresses α2-6 glycans with long branch topology. The alveolar tissue on the other hand predominantly expresses α2-3 glycans. H1 HA binds significantly to the apical surface of the trachea and its binding reduces gradually with dilution from 40 to 10 μg/ml (FIG. 19). H1 HA also shows some weak binding to the alveolar tissue only at the highest concentration. The binding pattern of H3 HA is different from that of H1 HA where in H3 HA shows significant binding to both tracheal and alveolar tissue sections at 40 and 20 μg/ml (FIG. 19). However, at a concentration of 10 μg/ml, the HA shows binding primarily to the apical side of the tracheal tissue and little to no binding to the alveolar tissue. Together, the tissue binding data point to 1) the high affinity binding of H1 and H3 HA to the apical side of the tracheal tissue and 2) while H3 HA shows affinity for α2-3 (relatively lower than α2-6) H1 HA is highly specific for α2-6.

The data in FIG. 19 were generated using the following methods. Formalin fixed and paraffin embedded human tissue lung and tracheal sections were purchased from US Biomax, Inc and from US Biological, respectively. Tissue sections were deparaffinized, rehydrated and incubated with 1% BSA in PBS for 30 minutes to prevent non-specific binding. H1N1 and H3N2 HA were pre-complexed with primary antibody (mouse anti 6×His tag, Abcam) and secondary antibody (Alexa fluor 488 goat anti mouse, Invitrogen) in a ratio of 4:2:1, respectively, for 20 minutes on ice. The complexes formed were diluted in 1% BSA-PBS to a final HA concentration of 40, 20 or 10 μg/ml. Tissue sections were then incubated with the HA-antibody complexes for 3 hours at RT. Sections were counterstained with propidium iodide (Invitrogen; 1:100 in TBST), washed extensively and then viewed under a confocal microscope (Zeiss LSM510 laser scanning confocal microscopy).

### Example 7

### Dose Response Direct Binding of Wild Type HA Polypeptides to Glycans of Different Topology

As described herein, the present invention encompasses the recognition that binding by HA polypeptides to glycans having a particular topology, herein termed “umbrella” topology, correlates with ability of the HA polypeptides to mediate infection of human hosts. The present Example describes results of direct binding studies with different HA polypeptides that mediate infection of different hosts, and illustrates the correlation between human infection and umbrella glycan binding.

Direct binding assays typically utilize glycan arrays in which defined glycan structures (e.g., monovalent or multivalent) are presented on a support (e.g., glass slides or well plates), often using a polymer backbone. In so-called “sequential” assays, trimeric HA polypeptide is bound to the array and then is detected, for example using labeled (e.g., with FITC or horse radish peroxidase) primary and secondary antibodies. In “multivalent” assays, trimeric HA is first complexed with primary and secondary antibodies (typically in a 4:2:1 HA:primary:secondary ratio), such that there are 12 glycan binding sites per pre-complexed HA, and is then contacted with the array. Binding assays are typically carried out over a range of HA concentrations, so that information is obtained regarding relative affinities for different glycans in the array.

For example, direct binding studies were performed with arrays having different glycans such as 3′SLN, 6′SLN, 3′SLN-LN, 6′SLN-LN, and 3′SLN-LN-LN, where LN represents Galβ1-4GlcNAc, 3′ represents Neu5Acα2-3, and 6′ represents Neu5Acα2-6). Specifically, biotinylated glycans (50 ul of 120 pmol/ml) were incubated overnight (in PBS at 4° C.) with a streptavidin-coated High Binding Capacity 384-well plate that was previously rinsed three times with PBS. The plate was then washed three times with PBS to remove excess glycan, and was used without further processing.

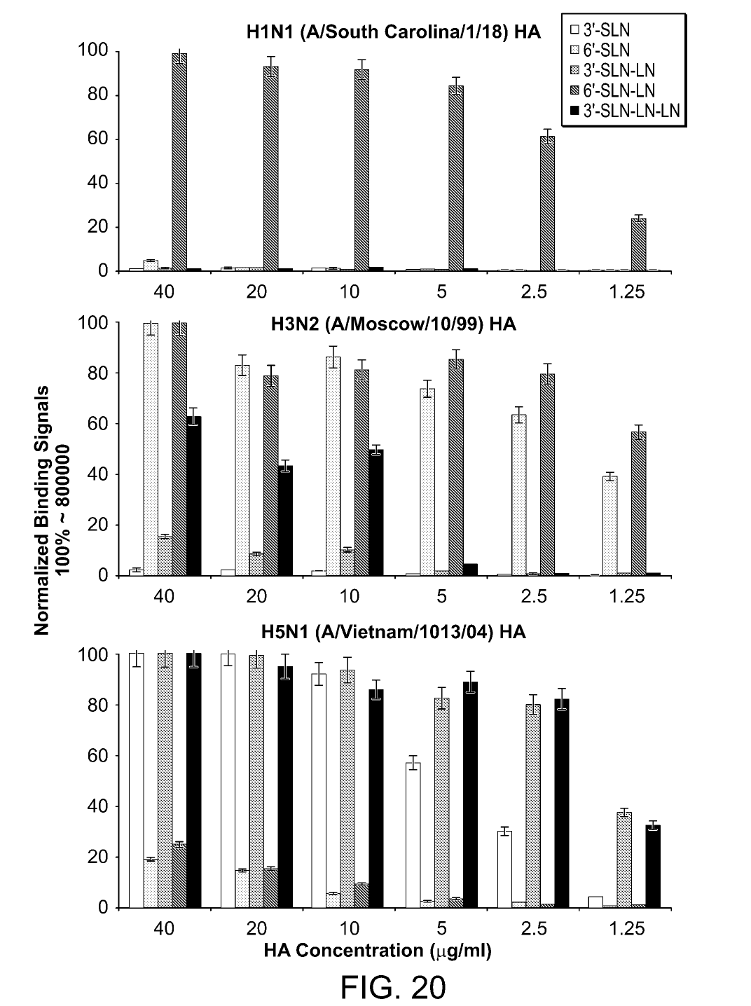

Appropriate amounts of His-tagged HA protein, primary antibody (mouse anti 6×His tag) and secondary antibody (HRP conjugated goat anti-mouse IgG) were incubated in a ratio of 4:2:1 HA:primary:secondary for 15 minutes on ice. The mixture (i.e., precomplexed HA) was then made up to a final volume of 250 ul with 1% BSA in PBS. 50 ul of the precomplexed HA was then added to the glycan-coated wells in the 384-well plate, and was incubated at room temperature for 2 hours. The wells were subsequently washed three times with PBS containing 0.05% TWEEN-20, and then three times with PBS. HRP activity was estimated using Amplex Red Peroxidase Kit (Invitrogen, CA) according to the manufacturer's instructions. Serial dilutions of HA precomplxes were studied. Appropriate negative (non-sialylated glycans) and background (no glycans or no HA) controls were included, and all assays were done in triplicate. Results are presented in FIG. 20

One characteristic of the binding pattern of known human adapted H1 and H3 HAs is their binding at saturating levels to the long α2-6 (6′SLN-LN) over a range of dilution from 40 down to 5 μg/ml (FIG. 20). While H1 HA is highly specific for binding to the long α2-6, H3 HA also binds to short α2-6 (6′SLN) with high affinity and to a long α2-3 with a lower affinity relative to α2-6 (FIG. 20). The direct binding dose response of H1 and H3 HA is consistent with the tissue binding pattern. Furthermore, the high affinity binding of H1 and H3 HA to long α2-6 correlates with their extensive binding to apical side of the tracheal tissues which expresses α2-6 glycans with long branch topology. This correlation provides valuable insights into the upper respiratory tissue tropism of human adapted H1 and H3 HAs. The tested H5 HA on the other hand shows the opposite glycan binding trend wherein it binds with high affinity to α2-3 (saturating signals from 40 down to 2.5 μg/ml) as compared to its relatively low affinity for α2-6 (significant signals seen only at 20-40 μg/ml) (FIG. 20).

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

TABLE 1
Features extracted from the glycans on the glycan array.
The features described in this table were used by the rule based classification
algorithm to identify patterns that characterized binding to specific GBP.

| Features extracted | Feature Description |
|---|---|
| Monosaccharide level | |
| Composition | Number of hex, hexNAcs, dHex, sialic acids, etc [In FIG. 1, the composition is |
| | Hex = 5; HexNAc = 4]. Terminal composition is distinctly recorded [In FIG. 1, the terminal |
| | composition is Hex = 2; HexNAc = 2]. |
| Explicit Composition | Number of Glc, Gal, GlcNAc, Fuc, GalNAc, Neu5Ac, Neu5Gc, etc [In FIG. 1, the explicit |
| | composition is Man = 5; GlcNAc = 4]. Terminal explicit composition is explicitly recorded [In |
| | FIG. 1, the terminal explicit composition is Man = 2; GlcNAc = 2]. |
| Higher order features | |
| Pairs | Pair refers to a pair of monosaccharide, connected covalently by a linkage. The pairs are |
| | classified into two categories, regular [B] and terminal [T] to distinguish between the |
| | pair with one monosaccharide that terminates in the non reducing end [FIG. 2]. The |
| | frequency of the pairs were extracted as features |
| Triplets | Triplet refers to a set of three monosaccharides connected covalently by two linkages. |
| | We classify them into three categories namely regular [B], terminal [T] and surface [S] |
| | [FIG. 2]. The compositions of each category of triplets were extracted as features |
| Quadruplets | Similar to the triplet features, quadruplets features are also extracted, with four |
| | monosaccharides and their linkages [FIG. 2]. Quadruplets are classified into two |
| | varieties regular [B] and surface [S]. The frequencies of the different quadruplets |
| | were extracted as features |
| Clusters | In the case of surface triplets and quadruplets above, if the linkage information is ignored, |
| | we get a set of monosaccharide clusters, and their frequency of occurrence (composition) |
| | is tabulated. These features were chosen to analyze the importance of types of linkages |
| | between the monosaccharides. |
| Average Leaf Depth | As an indicator of the effective length of the probes, average depth of the reducing end of |
| | the tree is extracted as a glycan feature. In FIG. 2B, the leaf depths are 3, 4 and 3, and |
| | the average is 3.34 |
| Number of Leaves | As a measure of spread of the glycan tree, the number of non reducing monosaccharides |
| | is extracted as a feature. For FIG. 2B, the number of leaves is 3. For FIG. 1 it is 4. |
| GBP binding features | These features are obtained for all GBPs screened using the array |
| Mean signal per glycan | Raw signal value averaged over triplicate or quadruplicate [depending on array version] |
| | representation of the same glycan |
| Signal to Noise Ratio | Mean noise computed based on negative control [standardized method developed by |
| | CFG] to calculate signal to noise ratio [S/N] |

TABLE 2
Crystal structures of HA-glycan complexes

| Abbreviation (PDB ID) | Virus strain | Glycan (with assigned coordinates) |
|---|---|---|
| ASI30_H1_23 (1RV0) | A/Swine/Iowa/30 (H1N1) | Neu5Ac |
| ASI30_H1_26 (1RVT) | A/Swine/Iowa/30 (H1N1) | Neu5Acα6Galβ4GlcNAcβ3Galβ4Glc |
| APR34_H1_23 (1RVX) | A/Puerto Rico/8/34 (H1N1) | Neu5Acα3Galβ4GlcNAc |
| APR34_H1_26 (1RVZ) | A/Puerto Rico/8/34 (H1N1) | Neu5Acα6Galβ4GlcNAc |
| ADU63_H3_23 (1MQM) | A/Duck/Ukraine/1/63 (H3N8) | Neu5Acα3Gal |
| ADU63_H3_26 (1MQN) | A/Duck/Ukraine/1/63 (H3N8) | Neu5Acα6Gal |
| AAI68_H3_23 (1HGG) | A/Aichi/2/68 (H3N2) | Neu5Acα3Galβ4Glc |
| ADS97_H5_23 (1JSN) | A/Duck/Singapore/3/97 (H5N3) | Neu5Acα3Galβ3GlcNAc |
| ADS97_H5_26 (1JSO) | A/Duck/Singapore/3/97 (H5N3) | Neu5Ac |
| Viet04_H5 (2FK0) | A/Vietnam/1203/2004 (H5N1) | |

The HA-α2-6 sialylated glycan complexes were generated by superimposition of the CA trace of the HA1 subunit of ADU63_H3 and ADS97_H5 and Viet04_H5 on ASI30_H1_26 and APR34_H1_26 (H1). Although the structural complexes of the human A/Aichi/2/68 (H3N2) with α2-6 sialylated glycans are published¹⁷, their coordinates were not available in the Protein Data Bank. The SARF2 (http://123d.ncifcrf.gov/sarf2.html) program was used to obtain the structural alignment of the different HA1 subunits for superimposition.

TABLE 3
Glycan receptor specificity of HAs based on classifier rules

| Influenza Strain | α2-3 Type^{a} | α2-6 Type^{b} |
|---|---|---|
| A/Duck/Alberta/35/76 (Avian H1N1) | 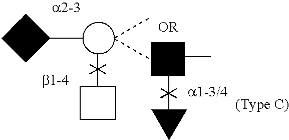 | 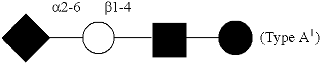 |
| A/Duck/Alberta/35/76 (Avian H1N1)Glu190Asp/Gly225 Asp double mutant | No | 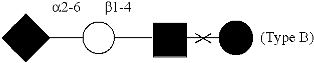 |
| A/South Carolina/1/18 (Human H1N1) | No | 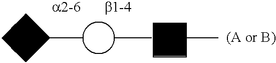 |
| A/New York/1/18 (Human H1N1) | 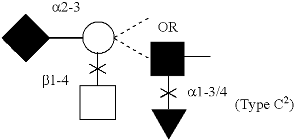 | 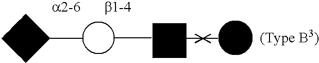 |
| A/Texas/36/91 (Human H1N1) | 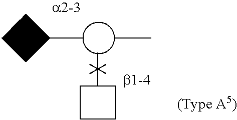 | 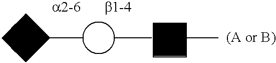 |
| A/New York/1/18 (Human H1N1)Asp 190Glu mutant⁴ | 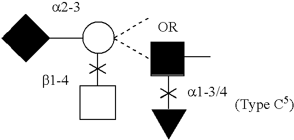 | 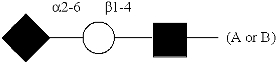 |
| A/New York/1/18 (Human H1N1) | No | No |
| Lys222 Leu mutant | | |
| A/Duck/Ukraine/1/63 (Avian H3N8) | 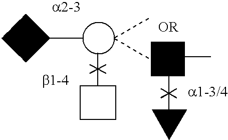 | No |
| A/Moscow/10/99 (Human H3N2) | No⁶ | 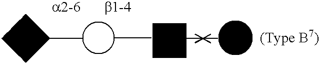 |
| A/Duck/Singapore/3/97 (Avian H5N3) | 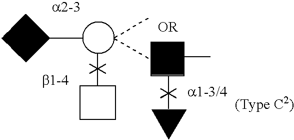 | No |
| A/Vietnam/1203/04 (Avian H5N1) | 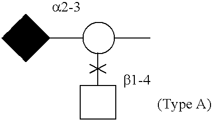 | No |
| A/Vietnam/1203/04 (Avian H5N1) | No | No |
| Glu 190Asp/Gly225 Asp double mutant | | |
| A/Vietnam/1203/04 (Avian H5N1)Gln226Leu/ Gly228Ser double mutant | 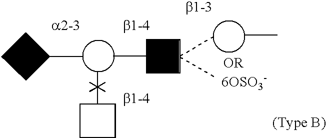 | 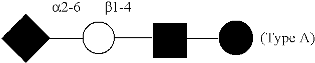 |
| A/Vietnam/1203/04 (Avian H5N1)Arg216Glu, Ser221 Pro double mutant | 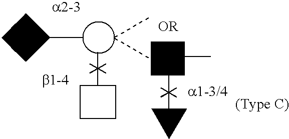 | No |

¹Border line high binder;
²Sulfated GlcNAc[6/S]/Gal[6S] high binders;
³Border line high) binders to a2-6 Type B. Only sulfated GlcNAc[6S]/Gal[6S] are high binders;
⁴Binds to several non-sialylated glycans;
⁵Border line high to α2-3 sialylated glycans;
⁶Few border line high binders to sulfated GlcNAc on Neu5Acα3Galβ3/4GlcNAc;
⁷High binders are Neu5Acα6Galβ4GlcNAcβ3Gal & !GlcNAcα6Man;
Others are boderline high.
Keys: [symbol] GlcNAc; [symbol] GalNAc; [symbol] Gal; [symbol] Man; [symbol] Fuc; [symbol] Neu5Ac;
The data from glycan microarray screening of H1, H3 and H5 subtypes were obtained from the Consortium for Functional Glycomics (CFG) web site-http://www.functionalglycomics.org/glycomics/publicdata/primaryscreen.jsp. The details of the data mining analysis including the description of features and classifiers are provided in Suppl FIG. 5. The rule induction classification method was used to generate the following classifiers (or rules) that govern the binding of HA to α2-3/6 sialylatedglycans. Classifiers for α2-3 sialylated glycan binding-Type A: Neu5Acα3Gal & !GalNAcβ4Gal, Type B: Neu5Acα3Galβ4GlcNAc & !GalNAcβ4Gal & {GlcNAcβ3Gal or GlcNAc[6S]}, Type C: Neu5AcαGalβ & !GalNAcβ4Gal & !Fucα3/4GlcNAc. Classifiers for α2-6 sialylated glycan binding-Type A: Neu5Acα6Galβ4GlcNAcb?Man, Type B: Neu5Acα6Galβ4GlcNAc & !GlcNAcb?Man. These complex rules graphically represented in the table for claarity. The rulesprovided as a logical combination of features among high affinity binders that enhance binding and features among weak and non-binders that are detrimental to binding (shown after the ‘!’ symbol in the text description and as a red linkage with a ‘x’ sign in the graphical representation). The presence of mannose in the α2-6 classifiers arises from the single 6′-silalyl lactosamine containing biantennary N-linked glycan on the glycan array.

We claim:

1. An engineered HA polypeptide that binds to umbrella-topology glycans.

2. The engineered HA polypeptide of claim 1, wherein the umbrella-topology glycans comprise α2-6 sialylated glycans.

3. The engineered HA polypeptide of claim 1 or claim 2, wherein the HA polypeptide binds to the umbrella-topology glycans with high affinity.

4. The engineered HA polypeptide of claim 3, wherein the HA polypeptide binds to the umbrella-topology glycans with an affinity comparable to that of a wild-type human adapted HA that mediates infection of humans.

5. The engineered HA polypeptide of claim 3, wherein the HA polypeptide binds to the umbrella-topology glycans with an affinity that is at least 50% that of a wild-type HA that mediates infection of humans.

6. The engineered HA polypeptide of claim 3, wherein the HA polypeptide binds to the umbrella-topology glycans with an affinity that is at least 70% that of a wild-type HA that mediates infection of humans.

7. The engineered HA polypeptide of claim 3, wherein the HA polypeptide binds to the umbrella-topology glycans with an affinity that is at least 80% that of a wild-type HA that mediates infection of humans.

8. The engineered HA polypeptide of claim 3, wherein the HA polypeptide binds to the umbrella-topology glycans with an affinity that is at least 90% that of a wild-type HA that mediates infection of humans.

9. The engineered HA polypeptide of claim 3, wherein the HA polypeptide binds to the umbrella-topology glycans with an affinity that is at least 100% that of a wild-type HA that mediates infection of humans.

10. The engineered HA polypeptide of claim 1 or claim 2, wherein the HA polypeptide binds to the umbrella-topology glycans preferentially as compared with cone-topology glycans.

11. The engineered HA polypeptide of claim 10, wherein the HA polypeptide binds to umbrella-topology glycans vs cone-topology glycans with a relative affinity of at least 2.

12. The engineered HA polypeptide of claim 10, wherein the HA polypeptide binds to umbrella-topology glycans vs cone-topology glycans with a relative affinity of at least 3.

13. The engineered HA polypeptide of claim 10, wherein the HA polypeptide binds to umbrella-topology glycans vs cone-topology glycans with a relative affinity of at least 4.

14. The engineered HA polypeptide of claim 10, wherein the HA polypeptide binds to umbrella-topology glycans vs cone-topology glycans with a relative affinity of at least 5.

15. The engineered HA polypeptide of claim 10, wherein the HA polypeptide binds to umbrella-topology glycans vs cone-topology glycans with a relative affinity of at least 10.

16. An isolated HA polypeptide that binds to umbrella-topology glycans other than, which HA polypeptide is not an H1 protein from any of the strains: A/South Carolina/1/1918; A/Puerto Rico/8/1934; A/Taiwan/1/1986; A/Texas/36/1991; A/Beijing/262/1995; A/Johannesburg/92/1996; A/New Caledonia/20/1999; A/Solomon Islands/3/2006, or an H2 protein from any of the strains: A/Japan/305+/1957; A/Singapore/1/1957; A/Taiwan/1/1964; A/Taiwan/1/1967, or an H3 protein from any of the strains: A/Aichi/2/1968; A/Phillipines/2/1982; A/Mississippi/1/1985; A/Leningrad/360/1986; A/Sichuan/2/1987; A/Shanghai/11/1987; A/Beijing/353/1989; A/Shandong/9/1993; A/Johannesburg/33/1994; A/Nanchang/813/1995; A/Sydney/5/1997; A/Moscow/10/1999; A/Panama/2007/1999; A/Wyoming/3/2003; A/Oklahoma/323/2003; A/California/7/2004; A/Wisconsin/65/2005.

17. A characteristic portion of an engineered HA polypeptide that binds to umbrella-topology glycans.

18. A characteristic portion of an HA polypeptide, which HA polypeptide is not an H1 protein from any of the strains: A/South Carolina/1/1918; A/Puerto Rico/8/1934; A/Taiwan/1/1986; A/Texas/36/1991; A/Beijing/262/1995; A/Johannesburg/92/1996; A/New Caledonia/20/1999; A/Solomon Islands/3/2006, or an H2 protein from any of the strains: A/Japan/305+/1957; A/Singapore/1/1957; A/Taiwan/1/1964; A/Taiwan/1/1967, or an H3 protein from any of the strains: A/Aichi/2/1968; A/Phillipines/2/1982; A/Mississippi/1/1985; A/Leningrad/360/1986; A/Sichuan/2/1987; A/Shanghai/11/1987; A/Beiging/353/1989; A/Shandong/9/1993; A/Johannesburg/33/1994; A/Nanchang/813/1995; A/Sydney/5/1997; A/Moscow/10/1999; A/Panama/2007/1999; A/Fujian/411/2002; A/Wyoming/3/2003; A/Oklahoma/323/2003; A/California/7/2004; A/Wisconsin/65/2005, wherein the characteristic portion binds to umbrella-topology glycans.

19. A polypeptide comprising the characteristic portion of claim 17 or claim 18.

20. A nucleic acid encoding the characteristic portion of claim 17 or claim 18.

21. A nucleic acid encoding the polypeptide of claim 19.

22. A vector containing the nucleic acid of claim 20.

23. A vector containing the nucleic acid of claim 21.

24. A host cell containing the nucleic acid of claim 20.

25. A host cell containing the nucleic acid of claim 21.

26. A host cell containing the vector of claim 22.

27. A host cell containing the vector of claim 23.

28. An antibody that binds to an engineered HA polypeptide that binds to umbrella-topology glycans.

29. An antibody that binds to an HA polypeptide, which HA polypeptide is not an H1 protein from any of the strains: A/South Carolina/1/1918; A/Puerto Rico/8/1934; A/Taiwan/1/1986; A/Texas/36/1991; A/Beijing/262/1995; A/Johannesburg/92/1996; A/New Caledonia/20/1999; A/Solomon Islands/3/2006, or an H2 protein from any of the strains: A/Japan/305+/1957; A/Singapore/1/1957; A/Taiwan/1/1964; A/Taiwan/1/1967, or an H3 protein from any of the strains: A/Aichi/2/1968; A/Phillipines/2/1982; A/Mississippi/1/1985; A/Leningrad/360/1986; A/Sichuan/2/1987; A/Shanghai/11/1987; A/Beiging/353/1989; A/Shandong/9/1993; A/Johannesburg/33/1994; A/Nanchang/813/1995; A/Sydney/5/1997; A/Moscow/10/1999; A/Panama/2007/1999; A/Fujian/411/2002; A/Wyoming/3/2003; A/Oklahoma/323/2003; A/California/7/2004; A/Wisconsin/65/2005, wherein the HA polypeptide binds to umbrella-topology glycans.

30. The antibody of claim 28 or claim 29, which antibody is polyclonal.

31. The antibody of claim 28 or claim 29, which antibody is monoclonal.

32. A viral particle including an engineered HA polypeptide that binds to umbrella-topology glycans.

33. A method of treating influenza infection by administering a composition comprising an engineered HA polypeptide that binds to umbrella-topology glycans, a polypeptide comprising a characteristic fragment of an engineered HA polypeptide that binds to umbrella-topology glycans, an antibody that binds to an engineered HA polypeptide that binds to umbrella-topology glycans, or characteristic portion thereof, a nucleic acid that encodes an engineered HA polypeptide that binds to umbrella-topology glycans or characteristic portion thereof, or combinations thereof.

34. A glycan array comprising glycan structures of at least about 10%, 15%, 20%, 25%, 30%, 35%, 40%, 45%, 50%, 55%, 60%, 65%, 70%, 75%, 80%, 85%, 90% 95%, or more of glycans found on HA receptors in human upper respiratory tract tissues.

35. A method for identifying or characterizing HA polypeptides, the method comprising steps of:
providing a sample containing an HA protein;
contacting the sample with the glycan array of claim 26; and
detecting binding of HA to one or more glycans on the array.


SEQUENCE LISTING (38 sequences)

SEQ ID NO: 1 - protein, 321 aa, Artificial Sequence
  NCBI influenza virus sequence
ENGTCYPGEFIDYEELREQLSSISSFEKFEIPKASSWPNHETTKGVTAACSYSGASSFYRNLLWITKKGTSYPKLSKSYTNNKGKEVLVWGVHHPPSVSEQQSLYQNADAYVSVGSSKYNRRFAPEIAARPEVRGQAGRMNYYWTLLDQGDTITFEATGNLIAPWYAFALNKGSDSGIITSDAPVHNCDTRCQTPHGALNSSLPFQNVHPITIGECPKYVKSTKLRMATGLRNVPSIQSRGLFGAIAGFIEGGWTGMIDGWYGYHHQNEQGSGYAADQKSTQNAIDGITSKVNSVIEKMNTQFTAVGKEFNNLERRIENLN

SEQ ID NO: 2 - protein, 323 aa, Artificial Sequence
  NCBI influenza virus sequence
ENGTCYPGDFIDYEELREQLSSVSSFEKFEIFPKTSSWPNHETTKGVTAACSYAGASSFYRNLLWLTKKGSSYPKLSKSYVNNKGKEVLVLWGVHHPPTGTDQQSLYQNADAYVSVGSSKYNRRFTPEIAARPKVRDQAGRMNYYWTLLEPGDTITFEATGNLIAPWYAFALNRGSGSGIITSDAPVHDCNTKCQTPHGAINSSLPFQNIHPVTIGECPKYVRSTKLRMATGLRNIPSIQSRGLFGAIAGFIEGGWTGMIDGWYGYHHQNEQGSGYAADQKSTANAIDGITNKVNSVIEKMNTQFTAVGKEFNNLERRIENLN

SEQ ID NO: 3 - protein, 323 aa, Artificial Sequence
  NCBI influenza virus sequence
ENGTCYPGYFADYEELREQLSSVSSFERFEIFPKESSWPNHTVTGVSASCSHNGKSSFYRNLLWLTGKNGLYPNLSKSYVNNKEKEVLVLWGVHHPPNIGDQRALYHTENAYVSVVSSHYSRRFTPEIAKRPKVRDQEGRINYYWTLLEPGDTIIFEANGNLIAPWYAFALSRGFGSGIITSNAPMDECDAKCQTPQGAINSSLPFQNVHPVTIGECPKYVRSAKLRMVTGLRNIPSIQSRGLFGAIAGFIEGGWTGMVDGWYGYHNNQNEQGSGYAADQKSTQNAINGITNKVNSVIEKMNTQFTAVGKEFNKLERRMENLN

SEQ ID NO: 4 - protein, 321 aa, Artificial Sequence
  NCBI influenza virus sequence
ANGLCYPGSFNDYEELKHLLTSVTHFEKVKILPRDQWTQHTTTGGSRACAVSGNPSFFRNMVWLTEKGSNYPIAKRSYNNTSGKQMLVIWGIHHPNDDTEQRTLYQNVGTYVSVGTSTLNKRSIPEIATRPKVNGQGGRMEFSWTLLETWDVINFESTGNLIAPEYGFKISKRGSSGIMKTEKTLENCETKCQTPLGAINTTLPFHNIHPLTIGECPKYVKSDRLVLATGLRNVPQIESRGLFGAIAGFIEGGWQGMVDGWYGYHHSNDQGSGYAADKESTQKAIDGITNKVNSVIEKMNTQFEAVGKEFNNLERRLENLN

SEQ ID NO: 5 - protein, 320 aa, Artificial Sequence
  NCBI influenza virus sequence
RDLCYPGSFNDYEELKHLLSSVKHFEKVKILPKDRWTQHTTTGGSRACAVSGNPSFFRNMVWLTEKGSNYPVAKGSYNNTSGEQMLIIWGVHHPNDEKEQRTLYQNVGTYVSVGTSTLNKRSTPDIATRPKVNGLGSRMEFSWTLLDMWDTINFESTGNLIAPEYGFKISKRGSSGIMKTEGTLENCETKCQTPLGAINTTLPFHNVHPLTIGECPKYVKSEKLVLATGLRNVPQIESRGLFGAIAGFIEGGWQGMIDGWYGYHHSNDQGSGYAADKESTQKAFDGITNKVNSVIEKMNTQFEAVGKEFSNLERRLENLN

SEQ ID NO: 6 - protein, 316 aa, Artificial Sequence
  NCBI influenza virus sequence
FSNCYPYDIPDYASLRSLVASSGTLEFITEGFTWTGVTQNGGSSACKRGPANGFFSRLNWLTKSESAYPVLNVTMPNNDNFDKLYIWGVHHPSTNQEQTDLYVQASGRVTVSTRRSQQTIIPNIGSRPWVRGQPGRISIYWTIVKPGDVLVINSNGNLIAPRGYFKMRTGKSSIMRSDAPIDTCISECITPNGSIPNDKPFQNVNKITYGACPKYVKNTLKLATGMRNVPGKQTRGLFGAIAGFIENGWEGMIDGWYGFRHQNSEGTGQAADLKSTQAAIDQINRKLNRVIEKTNEKFHQIEKEFSEVEGRIQDLE

SEQ ID NO: 7 - protein, 315 aa, Artificial Sequence
  NCBI influenza virus sequence
FSNCYPYDVPDYASLRSLVASSGTLEFITEGFTWTGVTQNGGSNACKRGPGSGFFSRLNWLTKSGSTYPVLNVTMPNNDNFDKLYIWGIHHPSTNQEQTSLYVQASGRVTVSTRRSQQTIIPNIGSRPWVRGLSSRISTYWTIVKPGDVLVINSNGNLIAPRGYFKMRTGKSSIMRSDAPIDTCISECITPNGSIPNKPFQNVNKITYGACPKYVKQNTLKLATGMRNVPEKQTRGLFGAIAGFIENGWEGMIDGWYGFRHQNSEGTGQAALKSTQAATDQINGKLNRVIEKTNEKFHQIEKEFSEVEGRIQDLE

SEQ ID NO: 8 - protein, 316 aa, Artificial Sequence
  NCBI influenza virus sequence
VSNCYPYDVPDYASLRSLVASSGTLEFNNESFNWAGVTQNGTSSACKRRSNKSFFSRLNWLTHLKYKYPALNVIMPNNEKFDKLYIWGVHHPVTDSDQISLYAQASGRITVSTKRSQQTVIPNIGYRPRVRDISSRISTYWTIVKPGDILLINSTGNLIAPRGYFKIRSGKSSIMRSDAPIGKCNSECITPNGSIPNDKPFQNVNRITYGACPRYVKQNTLKLATGMRNVPEKQTRGIFGAIAGFIENGWEGMVDGWYGFRHQNSEGTGQAADLKSTQAAINQINGKLRLIGKTNEKFHQIEKEFSEVEGRIQDLE

SEQ ID NO: 9 - protein, 319 aa, Artificial Sequence
  NCBI influenza virus sequence
VDTCYPFDVPDYQSLRSILANNGKFEFIAEEFQWNTVKQNGKSGACKRANVNDFFNRLNWLTKSNGDAYPLQNLTKVNNGDYARLYIWGVHHPSTDTEQTDLYKNNPGRVTVSTKTSQTSVVPNIGSRPWVRGQSGRISFYWTIVDPGDIIVFNTIGNLIAPRCHYKLNSQKKSTILNTAVPIGSCVSKCHTDRGSITTTKPFQNISRISIGDCPKYVKQGSLKLATGMRNIPEKATRGLFGAIAGFIENGWQGLIDGWYGFRHQNAEGTGTAADLKSTQAAIDQINGKLRNLIEKTNEKYHQIEKEFEQVEGRIQDLE

SEQ ID NO: 10 - protein, 324 aa, Artificial Sequence
  NCBI influenza virus sequence
VNDLCYPGDFNYEELKHLLSRINHFEKIQIIPKSSWSSHEASLGVSSACPYQGKSSFFRNVVWLIKKNSTYPTIKRSYNNTNQEDLLVLWGTHHPNDAAEQTKLYQNPTTYISVGTSTLNQRLVPRIATRSKVNGQSGRHEFFWTILKPNDINFESNGNFIAPEYAYKIVKKGDSTIMKSELEYGNCNTKCQTMGAINSSMPFHNIHPLTIGECPKYVKSNRLVLATGLRNSPQRERRRRKKRGLFGAIAGFIEGGWQGMVDGWYGYHHSNEQGSGYAADKESTQKAIDGVTNKVNSIIDKMNTQFEAVGREFNWLERRIENLN

SEQ ID NO: 11 - protein, 325 aa, Artificial Sequence
  NCBI influenza virus sequence
VNDLCYPGDFNYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPKIATRSKVNGQNGRMEFFWTILKPNDAINFESNGNFIAPEYAYKIVKKGDSAIMKSELEYGNCNTKCQTPMGAINSSMPFHNIHPLTIGECPKYVKNSRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLN

SEQ ID NO: 12 - protein, 325 aa, Artificial Sequence
  NCBI influenza virus sequence
QNGICYPGTLNEIEELKALIGSGERIERFEMFPKSTWSGVNTNNGVTRACPDNSGSSFYRNLLWITKTNSAAYPVIKGTYNNTGNQPILYFWGVHHPPDTNAQNNLYGSGDRYVRMGTESMNFAKGPEISARPVVNGQRGRIDYYWSVLKPGETLNVESNGNLIAPWYAYKFVSTNSKGAVFKSNLPIENCDATCQTTIAGVLRTNKTFQNVSPLWIGKCPKYVKSESLRLATGLRNVPQIATRGLFGAIAGFIEGGWTGLVDGWYGYHHENSQGSGYAADREATQKAIDGITNKVNSIIDKMNTQFEAVDHEFSNLERRIDNMN

SEQ ID NO: 13 - protein, 319 aa, Artificial Sequence
  NCBI influenza virus sequence
DSVCYPGKFVNEEALRQILRESGGINKETMGFTYSGIRTNGATSTCRRSGSSFYAEMKWLLSNTDNAAFPQMTKSYKNTRKDPALIIWGIHHSGSTTEQTKLYGSGNKLITVESSNYQQSFVPSPGARPKVDGQSGRIDFHWLMLNPNDTITFSFNGAFIAPDRASFLRGKSMGIQSGVQVDDNCEGDCYHSGGTIISNLPFQNINSRAVGKCPRYVKQESLMLATGMKNVPEIPKGRGLFGAIAGFIENGWEGLIDGWYGFRHQNAQGEGTAADYKSTQSAIDQITGKLNRLIEKTNQQFELIDNEFTEVEKQIGNVI

SEQ ID NO: 14 - protein, 325 aa, Artificial Sequence
  NCBI influenza virus sequence
PEGMCYPGSVENLEELRFVFSSAASYKRIRLFDYSRWNVTRSGTSKACNASTGGQSFYRSINWLTKKKPDTYDFNEGAYVNNEDGDIIFLWGIHHPPDTKEQTTLYKNANTLSSVTTNTINRSFQPNIGPRPLVRGQQGRMDYYWGILKRGETLKIRTNGNLIAPEFGYLLKGESYGRIIQNEDIPIGNCNTKCQTYAGAINSSKPFQNASHRHYMGECPKYVKKASLRLAVGLRNTPSVEPRGLFGAIAGFIEGGWSGMIDGWYGFHHSNESEGTGMAADQKSTQEAIDKITNKVNNIVDKMNREFEVVNHEFSEVEKRINMIN

SEQ ID NO: 15 - protein, 316 aa, Artificial Sequence
  NCBI influenza virus sequence
VNGTCYPGNVENLEELRTLFSSASSYQRIQIFPDTIWNVTVTGTSKACSGSFYRSMRWLTQKSGSYPVQDAQYTNNREKSILFVWGIHHPPTDTAWTNLYINTDTTTSVTTEDLNRIFKPVIGPRPLVNGLQGRINYYWSVLKPGQTLRVRSNGNLIAPWYGHVLSGGSHGRILKTDLNSGNCVVQCQTEKGGLNSTLPFHNISKYAFGICPKYVRVKSLKLAVGLRNVPARSNRGLFGAIAGFIEGGWPGLVAGWYGFQHSNDQGVGMAADRDSTQRAIDKITSKVNNIVDKMNKQYEIIDHEFSEVETRLNMIN

SEQ ID NO: 16 - protein, 321 aa, Artificial Sequence
  NCBI influenza virus sequence
IAYCYPGATVNEEALRQKIMESGGIDKISTGFTYGSSINSAGTTRSCMRSGGNSFYAELKWLVSKNKGQNFPQTANTYRNTDSAEHLIIWGIHHPSSTQEKNDLYGTQSLSISVGSSTYQNNFVPVVGARPQVNGQSGRIDFHWTMVQPGDNITFSHNGGLIAPSRVSKLKGRGLGIQSGASVDNDCESKCFWKGGSINTKLPFQNLSPRTVGQCPKYVNKKSLLLATGMRNVPEVVQGRGLFGAIAGFIENGWEGMVDGWYGFRHQNAQGTGQAADYKSTQAAIDQITGKLNRLIEKTNTEFESIESEFSEIEHQIGNVI

SEQ ID NO: 17 - protein, 321 aa, Artificial Sequence
  NCBI influenza virus sequence
TNGICYPTLENEEELRLKFSGVLEFSKFEAFTSNGWGAVNSGAGVTAACKFGSSNSFFRNMIWLIHQSGTYPVIRRTFNNTKGRDVLVVWGVHHPATLKEHQDLYKKDSSYVAVDSESYNRRFTPEISTRPKVNGQAGRMTFYWTIVKPGEAITESNGAFLAPRYAFELVSLGNGKLFRSDLNIESCSTKCQSEIGGINTNRSFHNVHRNTIGDCPKYVNVKSLKLATLGLRNVPAIATRGLFGAIAGFIEGGWPGLINGWYGFQHRNEEGTGIAADKESTQKAIDQITSKVNNIVDRMNTNFESVQHEFSEIEERINQLS

SEQ ID NO: 18 - protein, 320 aa, Artificial Sequence
  NCBI influenza virus sequence
MEGVCYPGSIENQEELRSLFSSIKKYERVKMFDFTKWNVTYTGTSRACNNTSNRGSFYRSMRWLTLKSGQFPVQTDEYKNTRDSDILFTWAIHHPPTSAEQVQLYKNPDTLSSVTTDEINRSFKPNIGPRPLVRGQQGRMDYYWAVLKPGQTKIGTNGNLIAPEYGHLITGKSHGRILKNNLPVGQCVTECQLNEGVMNTSKPFQNTSKHYIGKCPKYIPSGSLKLAIGLRNVPQVQNRGLFGAIAGFIEGGWPGLVAGWYGFQHQNAEGTGMAADRDSTQKAIDNMQNKLNNVIDKMNKQFEVVNHEFSEVESRINMIN

SEQ ID NO: 19 - protein, 318 aa, Artificial Sequence
  NCBI influenza virus sequence
PHGLCYPGELNNNGELRHLFSGIRSFSRTELIPPTSWGEVLDGATSARDDKGTNSFYRNLVWFVKKNNRYPVISKTNNTTGRVLVLWGIHHPVSVEETKTLYVNSDPYTLVSTKSWSEKYKLETGVRPGYNGQRSWMKIYWSLLHPGEMITFESNGGLLAPRYGYIIEEYGKGRIFQSRIRMSKCNTKCQTSVGGINTNRTFQNIDKNALGDCPKYIKSGQLKLATGLRNVPAIDNRGLLGAIAGFIEGGWPGLINGWYGFQHQNEQGTGIAADKESTQKAIDQITTKINNIIDKMNGNYDSIRGEFNQVEKRINMLA

SEQ ID NO: 20 - protein, 317 aa, Artificial Sequence
  NCBI influenza virus sequence
VDTCYPFDVPDYQSLRSILASSGSLEFIAEQFTWNGVKVDGSSSACLRGGRNSFFSRLNWLTKETNGNTGPINVTKENTGSYVRLYLWGVHHPSSDNEQTDLYKVATGRVTVSTRSDQISIVPNIGSRPRVRNQSGRISIYWTLVNPGDSIIFNSIGNLIAPRGHYKISKSTKSTVLKSDKRIGSCTSPCLTDKGSIQSDKPFQNVSRIAIGNCPKYVKQGSLMLATGMRNIPGKQAKGLFGAIAGFIENGWQGLIDWYGFRHQNAEGTGTAADLKSTQAAIDQINKLNRLIEKTNEKYHQIEKEFEQVEGRIQDLE

SEQ ID NO: 21 - protein, 327 aa, Artificial Sequence
  NCBI influenza virus sequence
SDICYPGKFTNEEALRQIIRESGGIDKEPMGFRYSGIKTDGATSACKRTVSSFYSEMKWLLSSKANQVFPQLQTYRNNRKEPALIVWGVHHSSSLDEQNKLYGAGNKLITVGSSKYQQSFSPSPDRPKVNGQAGRIDFHWMLLDPGDTVTFTFNGAFIAPDRATFLRSNAPSGVEYNGKSLGIQSDAQIDESCEGECFYSGGTINSPLPFQNIDSWAVGRCPRYVKQSSLPLALGMKNVPEKIHTRGLFGAIAGFIENGWEGLIDGWYGFRHQNAQGQGTAADYKSTQAAIDQITGKLNRLIEKTNTQFELIDNEFTEVEQQIGNVI

SEQ ID NO: 22 - protein, 320 aa, Artificial Sequence
  NCBI influenza virus sequence
PNKLCFRGELDNNGELRHLFSGVNSFSRTELISPNKWGDILDGVTASCRDNGASSFYRNLVWIVKNKNGKYPVIKGDYNNTTGRDVLVLWGIHHPDTETTAINLYASKNPYTLVSTKEWSKRYELEIGTRIGDGQRSWMKLYWHLMRPGERIMFESNGGLIAPRYGYIIEKYGTGRIFQSGVRMAKCNTKCQTSLGGINTNKTFQNIERNALGDCPKYIKSGQLKLATGLRNVPSVGERGLFGAIAGFIEGGWPGLINGWYGFQHQNEQGTGIAADKASTQKAIDEITTKINNIIEKMNGNYDSIRGEFNQVEKRINMLA

SEQ ID NO: 23 - protein, 164 aa, Artificial Sequence
  HA glycan binding domain sequence
SYIIETSNSENGTCYPGEFIDYEELREQLSSISSFEKFEIFPKASSWPNHETTKGVTAACSYSGASSFYRNLLWITKKGTSYPKLSKSYTNNKGKEVLVLWGVHHPPSVSEQQSLYQNADAYVSVGSSKYNRRFAPEIAARPEVRGQAGRMNYYWTLLDQGDTI

SEQ ID NO: 24 - protein, 165 aa, Artificial Sequence
  HA glycan binding domain sequence
SYIVETSNSDNGTCYPGDFIDYEELREQLSSVSSFEKFEIFPKTSSWPNHETTRGVTAACPYAGASSFYRNLLWLVKKGNSYPKLSKSYVNNNKGKEVLVLWGVHHPPTSTDQQSLYQNADAYVSVGSSKYDRRFTPEIAARPKVRGQAGRMNYYWTLLEPGDTI

SEQ ID NO: 25 - protein, 163 aa, Artificial Sequence
  HA glycan binding domain sequence
SYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTI

SEQ ID NO: 26 - protein, 164 aa, Artificial Sequence
  HA glycan binding domain sequence
SYIVETSNSENGTCYPGDFIDYEELREQLSSVSSFEKFEIFPKTSSWPNHETTKGVTAACSYAGASSFYRNLLWLTKKGSSYPKLSKSYVNNKGKEVLVLWGVHHPPTGTDQQSLYQNADAYVSVGSSKYNRRFTPEIAARPKVRDQAGRMNYYWTLLEPGDTI

SEQ ID NO: 27 - protein, 165 aa, Artificial Sequence
  HA glycan binding domain sequence
SYIAETPNPENGTCYPGYFADYEELREQLSSVSSFERFEIFPKESSWPNHTVTKGVTTSCSHNGKSSFYRNLLWLTKKNGLYPNVSKSYVNNKEKEVLVLWGVHHPSNIGDQRAIYHTENAYVSSVVSSHYSRRFTPEIAKRPKVRDQEGRINYYWTLLEPGDTI

SEQ ID NO: 28 - protein, 163 aa, Artificial Sequence
  HA glycan binding domain sequence
SYIETSNSENGTCYPGDFIDYEELREQLSSVSSFEKFEIFPKTSSWPNHETTKGVTAACSYAGASSFYRNLLWLTKKGSSYPKLSKSYVNNKGKEVLVLWGVHHPPTGTDQQSLYQNADAYVSVGSSKYNRRFTPEIAARPKVRGQAGRMNYYWTLLEPGDTI

SEQ ID NO: 29 - protein, 158 aa, Artificial Sequence
  HA glycan binding domain sequence
DLFVERSNAFSNCYPYDIPDYASRSLVASSGTLEFITEGFTWTGVTQNGGSSACKRGPANGFFSRLNWLTKSESAYPVLNVTMPNNDNFDKLYIWGVHHPSTNQEQTNLYVQASGRVTVSTRRSQQTIIPNIGSRPWVRGQPGRISIYWTIVKPGDVL

SEQ ID NO: 30 - protein, 159 aa, Artificial Sequence
  HA glycan binding domain sequence
DLFVERSKAFSNCYPYDVPDYASLRSLVASSGTLEFITEGFTWTGVTQNGGSNACKRGPGSGFFSRLNWLTKSGSTYPVLNVTMPNNDNFDKLYIWGIHHPSTNQEQTSLYVQASGRVTVSTRRSQQTIIPNIGSRPWVRGLSSRISIYWTIVKPGDVL

SEQ ID NO: 31 - protein, 157 aa, Artificial Sequence
  HA glycan binding domain sequence
DLFVERSKAYSNCYPYDVPDYASLRSLVASSGTLEFNNESFNWTGVANGTSSSCKRRSIKSFFSRLNWLHLKYRYPALNVTMPNNDKFDKLYIWGVHHPSTDSDQTSLYTQASGRVTVSTKRSQQTVIPNIGSRPWVRGISSRISIYWTIVKPGDLL

SEQ ID NO: 32 - protein, 163 aa, Artificial Sequence
  HA glycan binding domain sequence
SYIVEKDNPVNGLCYPENFNDYEELKHLLSSTNHFEKIRIIPRSSWSNHDASSGVSSACPYNGRSSFFRNVVWLIKKNNAYPTIKRSYNNTNQEDLLILWGIHHPNDAAEQTKLYQNPTTYVSVGTSTLNQRSVPEIATRPKVNGQSGRMEFFWTILKPNDAI

SEQ ID NO: 33 - protein, 162 aa, Artificial Sequence
  HA glycan binding domain sequence
SYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLQNPTTYISVGTSTLNQRLVPRIATRSKVNGQSGRMEFFWTIIKPNDAI

SEQ ID NO: 34 - protein, 570 aa, Artificial Sequence
  Sequence alignment illustrating conserved subsequences characteristic of H5 HA.
MEKIVLLLAIVSLVKSDQICIGYHANNSTEQVDTIMEKNVTVTHAQDILEKTHNGKLCDLDGVKPLILRDCSVAGWLLGNPMCDEFINVPEWSYIVEKANPANDLYCYPGDFNDYEELKHLLSRINHFEKIQIIPKSSWSDHEASSGVSSACPYQGKSSFFRNVVWLIKKNSAYPTIKKRSYNNTNQEDLLVLWGIHHPNDAAEQTKLYQNPTTYISVGTSTLNQRLVPKIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGTYQILSIYSTVASSLALAIMVAGLSLWMCSNGSLQCRICI

SEQ ID NO: 35 - protein, 570 aa, Artificial Sequence
  Sequence alignment illustrating conserved subsequences characteristic of H5 HA.
MEKIVLLLAIVSLVKSDQICIGYHANNSTEQVDTIMEKNVTVTHAQDILEKTHNGKLCDLDGVKPLILRDCSVAGWLLGNPMCDEFINVPEWSYIVEKASPDNDLYCYPGDFNDYEELKHLLSRINHFEKIQIIPKSSWSNHEASSGVSSACPYHGKSSFFRNVVWLIKKNSAYPTIKKRSYNNTNQEDLLVLWGIHHPNDAAEQTKLYQNPTTYISVGTSTLNQRLVPKIATRSKVNGQSGRMEFFWTILKPNDAINFESNGNFIAPEYAYKIVKKGDSAIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNKLERRIENLNKKMEDGFLDVWTYNAELLVLMENERTLDFHDSNVKNLYDKVRLQLRDNAKELGNGCFEFYHKCDNECMESVKNGTYDYPQYSEEARLNREEMSGVKLESIGTYQILSIYSTVASSLALAIMVAGLSLWMCSNGSLQCRICI

SEQ ID NO: 36 - protein, 570 aa, Artificial Sequence
  Sequence alignment illustrating conserved subsequences characteristic of H5 HA.
MERIVIALAIISIVKGDQICIGYHANNSTKQVDTIMEKNVTVTHAQDILEKEHNGKLCSLKGVRPLILKDCSVAGWLLGNPMCDEFLNVPEWSYIVEKDNPINGLYCYPGDFNDYEELKHLMSSTNHFEKIQIIPRSSWSNHDASSGVSSACPYNGRSSFFRNVVWLIKKNNAYPTIKKRTYNNTNQEDLLVLWGIHHPNDAAEQTKLYQNPTTYISVGTSTLNQRLVPKIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGTYQILSIYSTVASSLALAIMVAGLSLWMCSNGSLQCRICI

SEQ ID NO: 37 - protein, 570 aa, Artificial Sequence
  Sequence alignment illustrating conserved subsequences characteristic of H5 HA.
MEKIVLLFAIVSLVKSDQICIGYHANNSTEQVDTIMEKNVTVTHAQDILEKTHNGKLCDLDGVKPLILRDCSVAGWLLGNPMCDEFLNVPEWSYIVEKINPANDLYCYPGNFNDYEELKHLLSRINHFEKIQIIPKSSWSDHEASSGVSSACPYQGRSSFFRNVVWLIKKNDNYPTIKKRSYNNTNQEDLLVLWGIHHPNDAAEQTRLYQNPTTYISVGTSTLNQRLVPKIATRSKVNGQSGRMEFFWTILKSNDAINFESNGNFIAPENAYKIVKKGDSTIMKSELEYGNCNTKCQTPIGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHRCDNECMESVRNGTYDYPQYSEEARLKREEISGVKLESIGTYQILSIYSTVASSLALAIMVAGLFLWMCSNGSVTMRICI

SEQ ID NO: 38 - protein, 570 aa, Artificial Sequence
  Sequence alignment illustrating conserved subsequences characteristic of H5 HA.
MEKIVLLLAIVSLVKSDQICIGYHANNSTEQVDTIMEKNVTVTHAQDILEKTHNGKLCDLDGVKPLILRDCSVAGWLLGNPMCDEFINVPEWSYIVEKANPANDLYCYPGNFNDYEELKHLLSRINHFEKIQIIPKSSWSDHEASSGVSSACPYLGKSSFFRNVVWLIKKNSAYPTIKKRSYNNTNQEDLLVLWGIHHPNDAAEQTRLYQNPTTYISVGTSTLNQRLVPKIATRSKVNGQSGRMEFFWTILKPNDAINFESNGNFIAPEYAYKIVKKGDSAIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYNYPQYSEEARLKREEISGIKLESIGIYQILSIYSTVASSLALAIMVAGLSLWMCSNGSLQCRICI